(19) United States
(12) Patent Application Publication
(10) Pub. No.: US 2026/0298934 A1
(43) Pub. Date: Oct. 1, 2026

(54) SCREENING METHODS FOR ENDOMETRIAL CANCER

(71) Applicant: ARIZONA BOARD OF REGENTS ON BEHALF OF THE UNIVERSITY OF ARIZONA, Tucson, AZ (US)

(72) Inventors: Melissa M. Herbst-Kralovetz, Phoenix, AZ (US); Pawel Laniewski, Phoenix, AZ (US)

(21) Appl. No.: 19/701,482

(22) Filed: Jun. 8, 2026

Related U.S. Application Data
(63) Continuation-in-part of application No. PCT/US24/59234, filed on Dec. 9, 2024.
(60) Provisional application No. 63/607,724, filed on Dec. 8, 2023.
(60) Provisional application No. 63/613,339, filed on Dec. 21, 2023.

(51) Int. Cl. G01N 33/5755

(52) U.S. Cl. CPC G01N 33/5755

(57) ABSTRACT

Endometrial cancer (EC) is the most common gynecologic cancer in developed countries and the fourth most common cancer affecting women in the US. In contrast to other cancers, rates of EC continue to rise, and there are indications that social determinants of health and race and/or ethnicity contribute to risk. Thus, the methods described herein provide a non-invasive means of measuring biomarkers in the local cervicovaginal microenvironment. These novel biomarkers may be used for diagnosing and/or predicting women who are “at risk” for the development and progression of endometrial cancer (EC; e.g., EC type 1). Specifically, the methods herein may include obtaining a cervicovaginal lavage (CVL) or vaginal swab sample from a patient and producing a profile of at least five or more biomarkers from the collected sample.

### CROSS-REFERENCE TO RELATED APPLICATIONS

This application is a 371 and claims benefit of International Application No. PCT/US2024/059234 filed Dec. 9, 2024, which claims benefit of U.S. Provisional Application No. 63/613,339 filed Dec. 21, 2023, and U.S. Provisional Application No. 63/607,724 filed Dec. 8, 2023, the specifications of which are incorporated herein in their entirety by reference.

### FIELD OF THE INVENTION

The present invention relates to methods for predictive and diagnostic screening of women at risk for the development and progression of endometrial cancer. The methods feature detecting particular biomarkers using the local microenvironment.

### BACKGROUND OF THE INVENTION

Endometrial cancer (EC) is a cancer of the lining of the uterus, and is the most prevalent gynecologic malignancy in high-income countries, with an annual global estimated incidence of 417,000 new cases and 97,000 deaths. Risk factors of EC include increased body mass index (BMI), diabetes, older age, early menopause, hypertension, family history (Lynch syndrome), and polycystic ovary syndrome (PCOS). Atypical hyperplasia is also a major risk factor for the development of EC when left untreated, being an endometrial precancerous condition.

Current diagnosis of EC relies on histopathological investigation of biopsy samples. Endometrial specimens are typically collected at a physician's office using biopsy pipelles with or without hysteroscopy or dilation and curettage. However, certain populations of women (e.g., morbidly obese, mentally disabled, sexually traumatized) often require the operating room setting for biopsy sample collection. Imaging techniques, specifically transvaginal ultrasound, computed tomography (CT) scans, and magnetic resonance imaging (MRI), are used preoperatively in EC management. Through assessment of factors including myometrial invasion, lymphovascular invasion, and tumor size, these techniques are more reliable for pre-operative characterization of EC, as well as, for continually monitoring and management of EC; yet, they lack sensitivity and specificity for diagnostic value in EC. Regarding biomarkers, serum levels of two proteins: human epididymis protein 4 (HE4) and cancer antigen 125 (CA125), have been shown to be altered in EC, but these markers also lack sensitivity and specificity. Thus, additional research is needed to quantify protein biomarkers in the context of EC for sufficient diagnostic accuracy, preferably using samples collected by a non-invasive method.

### BRIEF SUMMARY OF THE INVENTION

It is an objective of the present invention to provide methods that allow for non-invasive point-of-care testing for early diagnosis of endometrial hyperplasia and cancer, as specified in the independent claims. Embodiments of the invention are given in the dependent claims. Embodiments of the present invention can be freely combined with each other if they are not mutually exclusive.

In some embodiments, the present invention features a method comprises obtaining a cervicovaginal lavage (CVL) sample from a patient, producing a profile of the CVL sample collected by detecting at least five or more metabolite biomarkers selected from one or a combination of: 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine; and measuring the CVL sample profile produced in the aforementioned step. In some embodiments, the metabolites are expressed in grade ½ endometrioid endometrial cancer (EEC). In some embodiments, the metabolite biomarkers detected in the CVL sample profile may further comprise one or more of AMP, spermine, myristoleoylcarnitine, heptadecasphingosine, myristoylcarnitine, pryridoxamine, 3-hydroxyhexanoate, GPC (16:0/20:3), palmitoleoylcarnitine, which may be expressed in all endometrial cancer (EMC). Alternatively, or in addition to, the metabolite biomarkers detected in the CVL sample profile may further comprise one or more of biliverdin, PC (P-16:0/20:4), 7-HOCA, PC (P-16:0/16:0), BHBA, X-25004, glycolithocholate sulfate, N-acetylserine, 3-hydroxyhexanoate, myristolycarnitine (C14:1), X-19913, which may be expressed in aggressive forms of endometrial cancer (EMC).

In some embodiments, the present invention features non-invasive method of diagnosing endometrial cancer (EC) in a subject in need thereof. In some embodiments, the method comprises determining the subject's levels of five or more metabolite biomarkers and diagnosing the patient with EC if the levels of at least five biomarkers are altered compared to a control profile. In some embodiments, determining the subject's levels of five or more metabolite biomarkers comprises obtaining a cervicovaginal lavage (CVL) sample from the subject; and measuring the levels of at least five or more metabolite biomarkers in the sample obtained; wherein the metabolite biomarkers are selected from one or a combination of: 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine. In certain embodiments, the subject is diagnosed with EC when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0), hexadecasphingosine (d16:1), X-17799, sphingadienine, or cholesterol sulfate are downregulated compared to a control profile and gamma-glutamylglutamine, 1-stearoyl-GPI (18:0), or cytidine 5′-monophosphate (5′-CMP) are upregulated compared to a control profile.

In some embodiments, the present invention may also feature an in vitro method of diagnosing endometrial cancer (EC) in a subject in need thereof. The method may comprise producing a profile from a vaginal swab sample having been obtained from a subject by detecting at least five or more metabolite biomarkers selected from one or a combination of: 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine, and diagnosing the patient with EC if the levels of at least five biomarkers are altered compared to a control profile. In some embodiments, the subject is diagnosed with EC if the levels of at least five biomarkers are altered compared to a control profile. The present invention may also feature methods of treating endometrial cancer (EC) in a subject in need thereof, where if a subject is diagnosed with EC, then an EC treatment is administered to the subject. In some embodiments, the the method diagnoses endometrial cancer (EC) in the subject, wherein a subject is diagnosed with EC with cancer when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are downregulated and gamma-glutamylglutamine, 1-stearoyl-GPI (18:0), or cytidine 5′-monophosphate (5′-CMP) are upregulated compared to a control profile.

In some embodiments, the present invention features a method of treating endometrial cancer (EC) in a patient in need thereof. The method may comprise diagnosing endometrial cancer (EC) in the patient as described herein and administering a therapeutic amount of a treatment to the patient if the patient is diagnosed with EC.

In other embodiments, the present invention features a method of monitoring an endometrial cancer treatment. In some embodiments, the method comprises obtaining a first cervicovaginal lavage (CVL) sample from the subject and producing a baseline profile of the CVL sample collected by detecting at least five or more metabolite biomarkers. For example, the baseline profile may be produced by detecting five or more metabolite biomarkers selected from one or a combination of: 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine. In some embodiments, the method comprises administering the treatment for EC to the subject. The method may further comprise obtaining a second cervicovaginal lavage (CVL) sample from the subject and producing a second profile of the CVL sample collected by detecting at least five or more metabolites biomarkers. For example, the second profile may be produced by detecting five or more metabolite biomarkers selected from one or a combination of: 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine. In some embodiments, the method comprises comparing the baseline profile of the CVL sample to the second profile of the CVL sample. In some embodiments, the treatment is effective if the levels of at least five biomarkers are altered from the baseline profile as compared to the second profile. In some embodiments, the treatment is effective when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are upregulated and gamma-glutamylglutamine, 1-stearoyl-GPI (18:0), or cytidine 5′-monophosphate (5′-CMP) are down-regulated.

In some embodiments, the present invention features a non-invasive method of determining a size of a tumor in a subject with endometrial cancer (EC). The method may comprise determining the patient's levels of five or more metabolites biomarkers by obtaining a cervicovaginal lavage (CVL) sample from the patien; and measuring the levels of five or more biomarkers in the sample obtained. In some embodiments, the five or more biomarkers comprise dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, and cytosine. The size of the tumor is greater than 2 cm if the levels of five or more biomarkers are altered compared to a predetermined threshold. In certain embodiments, dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) are upregulated in tumors greater than or equal to 2 cm and CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, cytosine are down-regulated in tumors greater than or equal to 2 cm.

In other embodiments, the present invention features a method may comprise obtaining a vaginal swab sample from a patient, producing a profile of the vaginal swab sample collected by detecting at least five or more metabolite biomarkers selected from one or more of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP) and analyzing the vaginal swab sample profile produced in the aforementioned step. In some embodiments, the metabolite biomarkers are expressed in grade ½ endometrioid endometrial cancer (EEC).

In some embodiments, the present invention features a non-invasive method of diagnosing endometrial cancer (EC) in a subject in need thereof. In some embodiments, the method comprises determining the patient's levels of five or more metabolite biomarkers and diagnosing the subject with EC if the levels of at least five biomarkers are altered compared to a control profile. In some embodiments, determining the subject's levels of five or more metabolite biomarkers comprises obtaining a vaginal swab sample from the patient; and measuring the levels of at least five or more metabolite biomarkers in the sample obtained; wherein the metabolite biomarkers are selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP). In some embodiments, the a subject is diagnosed with EC with cancer when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are downregulated and gamma-glutamylglutamine, 1-stearoyl-GPI (18:0), or cytidine 5′-monophosphate (5′-CMP) are upregulated.

In some embodiments, the present invention may also feature an in vitro method of diagnosing endometrial cancer (EC) in a subject in need thereof. The method may comprise producing a profile from a vaginal swab sample obtained from a subject by detecting at least five or more metabolite biomarkers selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP)and diagnosing the patient with EC if the levels of at least five biomarkers are altered compared to a control profile. In some embodiments, the subject is diagnosed with EC if the levels of at least five biomarkers are altered compared to a control profile. The present invention may also feature methods of treating endometrial cancer (EC) in a subject in need thereof, where if a subject is diagnosed with EC, then an EC treatment is administered to the subject. In some embodiments, the a subject is diagnosed with EC with cancer when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are downregulated and gamma-glutamylglutamine, 1-stearoyl-GPI (18:0), or cytidine 5′-monophosphate (5′-CMP) are upregulated.

In some embodiments, the present invention features a method of treating endometrial cancer (EC) in a patient in need thereof. The method may comprise diagnosing endometrial cancer (EC) in the patient as described herein and administering a therapeutic amount of a treatment to the patient if the patient is diagnosed with EC.

In other embodiments, the present invention features a method of monitoring an endometrial cancer treatment. In some embodiments, the method comprises obtaining a first vaginal swab sample from the subject and producing a baseline profile of the vaginal swab sample collected by detecting at least five or more metabolite biomarkers. For example, the baseline profile may be produced by detecting five or more metabolite biomarkers selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP). In some embodiments, the method comprises administering the treatment for EC to the subject. The method may further comprise obtaining a second vaginal swab sample from the subject and producing a second profile of the vaginal swab sample collected by detecting at least five or more metabolites biomarkers. For example, the second profile may be produced by detecting five or more metabolite biomarkers selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP). In some embodiments, the method comprises comparing the baseline profile of the vaginal swab sample to the second profile of the vaginal swab sample. In some embodiments, the treatment is effective if the levels of at least five biomarkers are altered from the baseline profile as compared to the second profile. In some embodiments, the treatment is effective when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are upregulated and gamma-glutamylglutamine, 1-stearoyl-GPI (18:0), or cytidine 5′-monophosphate (5′-CMP) are downregulated compared to a control profile.

In other embodiments, the present invention features a non-invasive method of determining a size of a tumor in a subject with endometrial cancer (EC). The method may comprise determining the patient's levels of five or more metabolites biomarkers by obtaining a vaginal swab sample from the patient, and measuring the levels of five or more biomarkers in the sample obtained. In some embodimetns, the five or more biomarkers comprise 7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991. The size of the tumor is greater than 2 cm if the levels of five or more biomarkers are altered compared to a predetermined threshold. In certain embodiments, 7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991 are down-regulated in tumors greater than or equal to 2 cm.

The present invention may further feature a non-invasive method of determining a prognosis of endometrial cancer (EC) in a subject in need thereof. In some embodiments, the method comprises characterizing endometrial tumor characteristics comprising tumor size, myometrial invasion, mismatch repair (MMR) status, histological grade, age, or a combination thereof and determining the prognosis of the patient. In some embodiments, the endometrial tumor characteristics may be determined by determining the patient's levels of five or more metabolites biomarkers by obtaining a biological sample from the patient; and measuring the levels of two or more biomarkers in the sample obtained. In some embodiments, a tumor size larger than 2 cm, presence of myometrial invasion, MMR proficient, and grade 3 is indicative of a poor prognosis and a tumor size smaller than 2 cm, no myometrial invasion, MMR deficient and grade ½ is indicative of a good prognosis. In some embodiments, the biological sample comprises a cervicovaginal lavage (CVL) sample, a urine sample, a vaginal swab, or a cervicovaginal secretion; wherein the cervicovaginal secretion is collected via a self collected lavage or a menstrual cup.

One of the unique and inventive technical features of the present invention is non-invasive sampling (e.g., a cervicovaginal lavage (CVL) or vaginal swab). Without wishing to limit the invention to any theory or mechanism, it is believed that the technical feature of the present invention advantageously provides for the detection of EC-related metabolite biomarkers in the cervicovaginal microenvironment. None of the presently known prior references or work has the unique, inventive technical feature of the present invention.

Furthermore, the prior references teach away from the present invention. For example, for a definitive diagnosis, women undergo various time-consuming and painful medical procedures, such as endometrial biopsy with or without hysteroscopy, and dilation and curettage, which may create a barrier to early detection and treatment, particularly for women with inadequate healthcare access. Specifically, invasive approaches create a barrier to screening, and there is currently no screening method for the early detection of EC in asymptomatic women.

Furthermore, the inventive technical features of the present invention contributed to a surprising result. Patients with tumors exhibiting myometrial invasion showed a significant increase in lipids, particularly glycerophospholipids, detected in vaginal swabs. These lipid profiles were distinct from those identified in cervicovaginal lavage (CVL) samples. Additionally, patients with larger tumors and MMR-deficient tumors demonstrated a depletion of various metabolites, including lipids and other metabolite classes, in vaginal swabs, while only two metabolites varied between histological subtypes in these samples. Overall, vaginal swabs revealed unique but less abundant metabolic features for differentiating tumor subtypes compared to CVL samples, suggesting that CVL metabolites may have greater potential for endometrial cancer (EC) stratification.

Another surprising result of the present invention contributed to a surprising result is that the targets that were most predictive were not the targets that were anticipated or predicted would be most predictive of disease status. Additional multivariate biomarker discovery analysis also yielded a unique set of targets that, when combined, were most predictive of disease status.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWINGS

The patent or application file contains at least one drawing executed in color. Copies of this patent or patent application publication with color drawings will be provided by the Office upon request and payment of the necessary fee.

The features and advantages of the present invention will become apparent from a consideration of the following detailed description presented in connection with the accompanying drawings in which:

FIGS. 1A, 1B, 1C, and 1D show metabolic analysis reveals the upregulation of lipids and downregulation of amino acids in endometrial cancer compared to benign conditions. FIG. 1A shows a pie chart representing the proportions of different classes of lipids according to all detected lipids (n=228) in all samples (n=192). FIG. 1B shows bar charts showing significantly altered (q<0.05, FC>2.0), FDR-corrected lipids in EC All, grade ½ EEC, and other EC compared to benign controls. Color-coded by lipid class. FIG. 1C shows a pie chart representing the proportions of different classes of amino acids according to all detected amino acids (n=206) in all samples (n=192). FIG. 1D shows bar charts showing significantly altered (q<0.05, FC>2.0), FDR-corrected amino acids in EC All, grade ½ EEC, and other EC compared to benign controls. Color-coded by amino acid class. This is important because there are particular subpathways of each of the lipid and amino acid groups.

FIGS. 2A, 2B, 2C, and 2D shows the fold change and T-test data reveals significant (q<0.05, FC>2.0) up/downregulation of metabolites in endometrial cancer compared to benign controls. FIGS. 2A and 2B show Volcano plots showing FDR-corrected, significantly altered (q<0.05, FC>2.0) metabolites in grade ½ EEC (FIG. 2A) and other EC (FIG. 2B) compared to benign controls. Color-coded by significance and up/downregulation. Top altered metabolites are labeled. Uncharacterized metabolites were not included in this analysis. FIG. 2C shows a Venn diagram comparing all significantly altered (q<0.05, FC>2.0) metabolites in grade ½ EEC and other EC compared to benign controls. Bar plot demonstrates the superpathway profiles of each section from the Venn diagram. FIG. 2D shows bar charts representing downregulated amino acid and upregulated lipid classes unique to each cancer type and shared between all endometrial cancers. This is important because the specific metabolites and subpathways were identified that are altered and more indicative of grade ½ EEC and other EC vs. non-malignant controls.

FIG. 3 shows the top 25 enriched pathways in EC All vs non-malignant controls. Enrichment analysis is based on the KEGG pathway database. This is important because it revealed metabolic pathways altered in endometrial cancer vs non-malignant controls.

FIGS. 4A, 4B, and 4C show receiver operating characteristic (ROC) analysis reveals a multitude of potential biomarkers to detect endometrial cancer patients from benign controls. FIG. 4A shows a scatter plot showing 10 metabolites had an AUC>0.8 (good biomarker threshold) for EC All vs benign controls. Metabolites color-coded by superpathway. Uncharacterized metabolites not included in analysis. FIG. 4B shows a scatter plot showing the AUC values of the 10 metabolites from panel A for grade ½ EEC and other EC vs benign controls. Metabolites color-coded by superpathway. FIG. 4C shows AUC plots for the top four potential EC All biomarkers showing the AUC values for grade ½ EEC and other EC vs benign controls. This is important because it demonstrates that these specific and individual metabolites are sensitive and specific for prediction of all types of endometrial cancer and then specifically for grade ½ EEC and other EC relative to non-malignant controls.

FIGS. 5A and 5B show the top 15 metabolites with biomarker potential for other EC vs non-malignant controls (AUC>0.8 is considered good and AUC>0.9 is considered excellent) by receiver operating characteristic (ROC) analysis. The analysis revealed a multitude of potential biomarkers to detect other EC patients from benign controls. FIG. 6A shows metabolites color-coded by superpathway. Uncharacterized metabolites were included in analysis. FIG. 6B shows AUC plots for the top three potential other EC biomarkers showing the AUC values. This is important because it demonstrates unique and individual metabolites that are specific for other endometrial cancer types that are higher grade and more aggressive subtype of endometrial cancer.

FIGS. 6A, 6B, 6C, and 6D show multivariate ROC analysis reveals potential for a multiple metabolites test to distinguish endometrial cancer patients from benign controls. FIG. 6A shows AUC plot from multivariate analysis, color-coded by the number of metabolites. Metabolites were chosen for analysis by random forest machine learning. Uncharacterized metabolites were not included in the analysis. Var. =number of variables (metabolites). AUC=area under curve. Range=AUC range. PA=predictive accuracy. FIG. 6B shows the top 15 selected metabolites (based on random forest) and their selected frequency. Color-coded by superpathway. Squares represent the relative levels of each metabolite in EC All and benign. FIG. 6C shows a cross validation plot demonstrating the ability of multivariate ROC to predict disease groups. FIG. 6D shows a confusion matrix demonstrating the percentage of samples predicted correctly/incorrectly. Color-coded by percentage of total samples per disease group. This is important because combining multiple metabolites in this analysis demonstrated an increase in predictive accuracy for all endometrial cancer types.

FIGS. 7A, 7B, and 7C show analysis of pathology data reveals relationships between metabolites and tumor characteristics. FIG. 7A shows fold-change and T-test data combined to produce volcano plots show significantly (p<0.05) up/downregulated (FC>2.0) metabolites associated with histological grade (other EC vs grade ½ EEC), MMR status (MMR deficient vs MMR proficient), myometrial invasion (present vs not present), and tumor size (>2 cm vs 2 cm). FIG. 7B shows Spearman correlation analysis shows top 20 significantly (p<0.05) correlated metabolites with increased tumor size and increased depth of myometrial invasion. FIG. 7C shows Venn diagram displaying number of significantly altered (p<0.05, FC>2.0) metabolites that are unique or shared among the different tumor characteristics. This is important because using the CVL we were able to predict tumor size prior to surgery as well as other tumor characteristics such as histological grade, MMR status and myometrial invasion using these metabolic markers. Some markers are shared among these features and others are unique.

FIGS. 8A, 8B, 8C, and 8D shows metabolic profiles of vaginal swab samples substantially differ from metabolic profiles of cervicovaginal lavage samples. FIGS. 8A and 8B shows the total number of metabolites belonging to different superpathways detected in vaginal swab or CVL samples (FIG. 8A) and the number of unique metabolites detected in each sample type and overlapped metabolites detected in both sample types (FIG. 8B). FIGS. 8C and 8D shows the total number of metabolites belonging to the lipid superpathway detected in vaginal swab or CVL samples (FIG. 8C) and the number of unique lipids detected in each sample type and overlapped lipids detected in both sample types (FIG. 8D).

FIGS. 9A, 9B, and 9C shows lipid, amino acid and peptide metabolism is significantly dysregulated in endometrial cancer compared to benign conditions based on the vaginal swab profiles. FIG. 9A shows the number of significantly altered metabolites between the endometrial cancer and benign groups with minimum fold change (FC) difference of 2 (FC>2 or <−2). Statistical difference between the groups was determined using unpaired t-test with false discovery rate (FDR) correction. Metabolites with q<0.05 and FC>2 or FC<−2 were considered significant and were colored based on FC differences. The top enriched and depleted metabolites were also labeled. FIG. 9B shows the number of significantly depleted and enriched metabolites in endometrial cancer compared to benign controls. Metabolites were grouped based on the superpathways. The altered metabolites mostly belonged to lipid, amino acid, uncharacterized, and peptide superpathways. FIG. 9C shows a bubble plot represents the top 25 enriched sets of metabolites in endometrial cancer compared to benign controls identified using the metabolite set enrichment analysis (MSEA). Bubble size and color indicate the enrichment ratio and p value, respectively.

FIGS. 10A, 10B, 10C, and 10D shows metabolites detected in vaginal swabs differentiate patients based on the endometrial tumor characteristics indicating potential prognostic utility. Volcano plot shows differences in metabolite levels in vaginal swab samples collected from patients with endometrial cancer and stratified based on tumor characteristics, including histological type (other EC vs. grade ½ EEC; FIG. 10A), tumor size (>2 cm vs. ≤2 cm; FIG. 10B), myometrial invasion (present vs. absent; FIG. 10C), and mismatch repair (MMR) status (MMR-proficient vs. MMR-deficient; FIG. 10D). Statistical differences between the groups were determined using unpaired t-test. Metabolites with p<0.05 and FC>2 were considered significant and were colored based on FC differences. The top altered metabolites were also labeled.

FIGS. 11A and 11B shows the top predictive metabolic biomarkers for endometrial cancer differ between vaginal swab and CVL samples. A receiver operating characteristic (ROC) analysis was used to identify vaginal swab and CVL metabolites discriminating endometrial cancer from benign controls. The area under curve (AUC) values were used to measure the discriminatory potential of identified biomarkers. FIG. 11A shows the top 15 metabolites with the highest AUC values for metabolites detected in vaginal swabs and corresponding metabolites in CVL samples. FIG. 11B shows the top 15 metabolites with the highest AUC values for metabolites detected in CVL samples and corresponding metabolites in vaginal swabs samples. Color of a dot indicates the specimen type and color-coded squares represent the metabolic superpathways. Metabolites with AUC>0.8 were considered to have good discriminatory potential for tested disease groups.

FIGS. 12A, 12B, 12C, 12D, 12E, and 12F shows the most predictive multivariable models distinguishing endometrial cancer from benign controls utilize different combinations of metabolites in vaginal swab and CVL samples. The least absolute shrinkage and selection operator (LASSO) algorithm was utilized to identify important features from metabolite sets detected in each sample type. Logistic regression models with a various number of features were built and evaluated by Monte Carlo cross-validation. FIG. 12A shows the best-performing model for vaginal swabs consisted of 13 features. A multivariable ROC analysis resulted in AUC of 0.868 indicating a good prediction of endometrial cancer when compared to benign controls by the vaginal swab model. FIG. 12B shows the confusion matrix shows the vaginal swab model correctly classifying 143 out of 174 tested samples (82.2%). FIG. 12C shows a dot plot that presents a list of 13 metabolic features used to build the vaginal swab model with the corresponding LASSO frequencies and metabolic pathways indicated by colored dots. FIG. 12D shows the best-performing model for CVL samples consisted of 17 features. A multivariable ROC analysis resulted in AUC of 0.905 indicating an excellent prediction of disease groups by the CVL model. FIG. 12E shows the confusion matrix shows the CVL model correctly classifying 146 out of 174 tested samples (83.9%). FIG. 12F shows a dot plot that presents a list of 17 metabolic features used to build the CVL swab model with the corresponding LASSO frequencies and metabolic pathways indicated by colored dots.

### DETAILED DESCRIPTION OF THE INVENTION

The following abbreviations are used throughout: 3-CMPFP, 3-carboxy-4-methyl-5-pentyl-2-furanpropionate; 6-OPCA, 6-oxopiperidine-2-carboxylate; 18:0-18:1 PS, 1-stearoyl-2-oleoyl-glycerophosphoserine (18:0/18:1); AUC, area under the curve; BHBA, 3-hydroxybutyrate; BMI, body mass index; Cer, ceramide; LacCer, lactosylceramide; SM, sphingomyelin; CMP, cytidine 5′-monophosphate; CVL, cervicovaginal lavage; Cys-Gly, oxidized, cystinyl-bis-glycine; EC, endometrial cancer; EEC, endometrioid carcinoma; ESI, electrospray ionization; FC, fold change; FDR, false discovery rate; γ-Glu-Gln, γ-glutamylglutamine; Gln-C6H1002(2), glutamine conjugate of C6H1002; GPC, glycerophosphorylcholine; GPEA, glycerophosphoethanolamine; GPG, glycerophosphoglycerol; GPSer, glycerophosphoserine; GPI, glycerophosphoinositol; HCA, hierarchical clustering analysis; J, Youden's index; LASSO, least absolute shrinkage and selection operator; Leu-Glu, leucylglutamate; LPI, lysophosphatidylinositol; MAG, monoacylglycerol; Mha acid, 2-hydroxy-4-(methylthio)butanoic acid; MSEA, metabolite set enrichment analysis; PC, phosphatidylcholine; PE, phosphatidylethanolamine; Phe-Gly, phenylalanylglycine; PLA, phenyllactate; PLS-DA, partial least squares discriminant analysis; Pro-Gly, prolylglycine; QC, quality control; RI, retention index; ROC, receiver operating characteristic; RP, reverse phase; RSD, relative standard deviation; SAH, S-adenosylhomocysteine; SAM, S-adenosylmethionine; SMPDB, Small Molecule Pathway Database; UDPGNAc, UDP-N-acetylglucosamine/galactosamine; UMP, uridine 5′-monophosphate; UPLC-MS/MS, ultra-performance liquid chromatography-tandem mass spectrometry; Val-Gly, valylglycine; and Vitamin B5, pantothenate.

Lipid species are designated according to standard lipidomics nomenclature, wherein the lipid class is followed by the fatty acyl and/or sphingoid chain composition. Representative lipid species analyzed include ceramides (Cer), lactosylceramides (LacCer), sphingomyelins (SM), phosphatidylcholines (PC), phosphatidylethanolamines (PE), phosphatidylinositols (PI), phosphatidylserines (PS), lysophosphatidylinositols (LPI), monoacylglycerols (MAG), and related metabolites.

### TERMS

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

The term “cancer” refers to any physiological condition in mammals characterized by unregulated cell growth. Cancers described herein include solid tumors. A “solid tumor” or “tumor” refers to a lesion and neoplastic cell growth and proliferation, whether malignant or benign and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The term “hyperplasia” may refer to when healthy cells undergo abnormal changes within tissues or organs, and it is considered a pre-cancerous disease state. In some embodiments, hyperplasia may progress and become cancer. In other embodiments, hyperplasia may regress. The term “pre-cancerous disease state” may refer to a condition or lesion involving abnormal cells associated with an increased risk of developing into cancer. In some embodiments, the progression of normal cells to precancerous cells and towards endometrial cancer may involve oncogenes, inflammation, and multiple somatic mutations that initiate the malignant transformation, activation, and clonal expansion of stem cells.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder, or condition described herein. In another embodiment, the subject is a mammal (e.g., a human) at risk of developing a disease, disorder, or condition described herein. In certain instances, the term patient refers to a human.

As used herein, the terms “normal subject,” “benign control,” “non-malignant” control,” or “control subject” may be used interchangeably and refers to a subject with benign gynecologic conditions. In some embodiments, a normal subject may refer to a subject undergoing a hysterectomy for a benign condition, e.g., abnormal uterine bleeding, endometriosis, pelvic pain, etc.

The terms “polypeptide” and “protein” are used interchangeably to refer to a polymer of amino acid residues, comprising natural or non-natural amino acid residues, and are not limited to a minimum length. Thus, peptides, oligopeptides, dimers, multimers, and the like are included within the definition. Both full-length proteins and fragments thereof are encompassed by the definition.

As used herein, the term “peptide” refers to a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (synthetic) sequence.

As used herein, the term “metabolite” refers to a small molecule that is an intermediate or end product of cellular metabolism within a living organism. These molecules play crucial roles in various biological processes, including but not limited to, energy production, cellular signaling, and the synthesis of essential biomolecules such as amino acids, nucleotides, and lipids. Metabolites encompass both primary metabolites, directly involved in fundamental cellular functions, and secondary metabolites, which may serve specialized functions such as defense mechanisms or environmental adaptation.

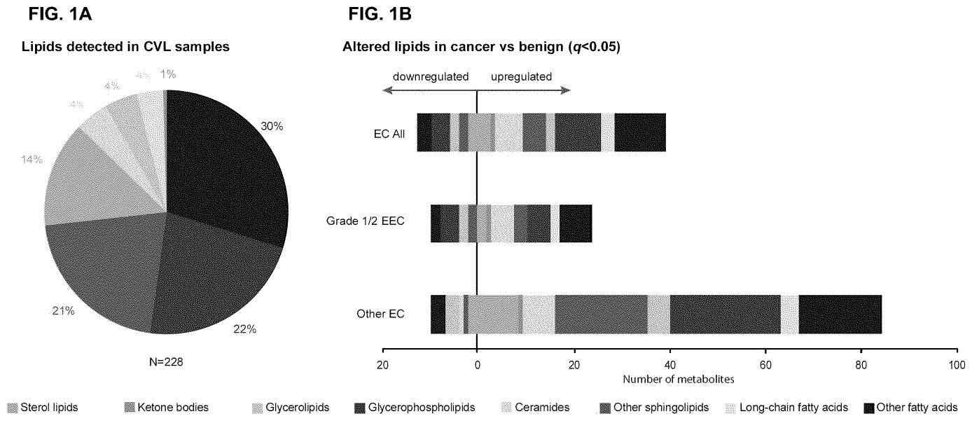

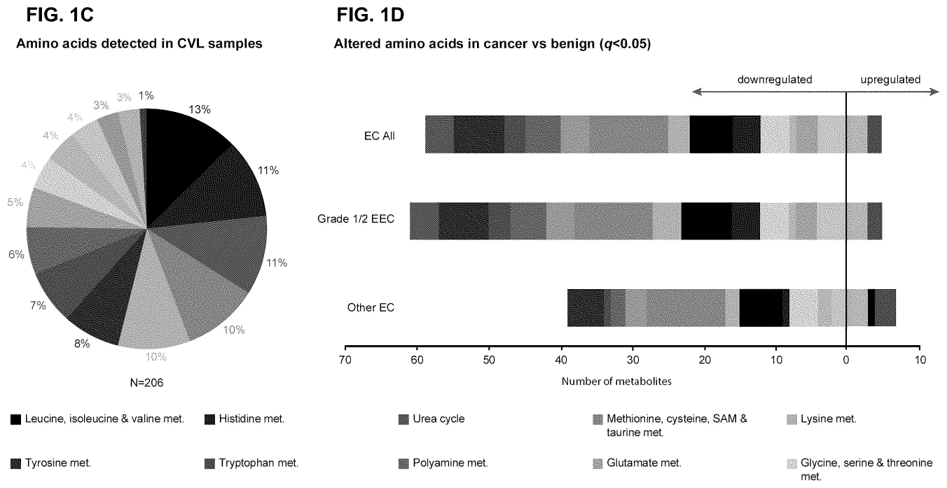

Referring now to FIGS. 1A-12F, the present invention features methods (e.g., non/minimally invasive methods) for improving early EC detection/diagnosis among diverse racial and ethnic populations by developing cost-effective, robust, non-invasive diagnostics that facilitate a better understanding and decrease morbidity associated with this cancer health disparity in women.

In some embodiments, the present invention features a method comprising obtaining a biological sample (e.g., a cervicovaginal sample) from a patient, producing a profile of the aforementioned biological sample (e.g., a cervicovaginal sample) collected by detecting at least five or more metabolite biomarkers, and measuring the biological sample (e.g., a cervicovaginal sample) profile produced. Non-limiting examples of cervicovaginal samples include samples obtained from the vagina and/or cervix, including, but not limited to, vaginal swabs, cervical swabs, cervicovaginal lavage (CVL) samples, vaginal washes, aspirates, secretions, and combinations thereof. In certain embodiments, the present invention features a method comprising obtaining a cervicovaginal lavage (CVL) sample from a patient, producing a profile of the aforementioned CVL sample collected by detecting at least five or more metabolite biomarkers; and measuring the CVL sample profile produced. In other embodiments, the present invention features a method comprising obtaining a vaginal swab sample from a patient, producing a profile of the aforementioned vaginal swab sample collected by detecting at least five or more metabolite biomarkers; and measuring the vaginal swab sample profile produced.

In some embodiments, the method comprises obtaining a cervicovaginal lavage (CVL) sample from a patient, producing a profile of the CVL sample collected by detecting at least five or more metabolite biomarkers selected from one or a combination of: 6-oxopiperidine-2-carboxylate, glycerophosphoethanolamine (GPEA), glycerophosphocholine (GPC), guanine, cytosine, glycerophosphoserine, X-19913, X-24724, lyxonate, prolylglycine, glycerophosphoglycerol, or N-acetylserine; and measuring the CVL sample profile produced in the aforementioned step. In some embodiments, the metabolites are expressed in grade ½ endometrioid endometrial cancer (EEC). In some embodiments, the metabolite biomarkers detected in the CVL sample profile may further comprise one or more of AMP, spermine, myristoleoylcarnitine, heptadecasphingosine, myristoylcarnitine, pryridoxamine, 3-hydroxyhexanoate, GPC (16:0/20:3), palmitoleoylcarnitine, which may be expressed in all endometrial cancer (EMC). Alternatively, or in addition to, the metabolite biomarkers detected in the CVL sample profile may further comprise one or more of biliverdin, PC (P-16:0/20:4), 7-HOCA, PC (P-16:0/16:0), BHBA, X-25004, glycolithocholate sulfate, N-acetylserine, 3-hydroxyhexanoate, myristolycarnitine (C14:1), X-19913, which may be expressed in aggressive forms of endometrial cancer (EMC).

In other embodiments, the method may comprise obtaining a vaginal swab sample from a patient, producing a profile of the vaginal swab sample collected by detecting at least five or more metabolite biomarkers selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP) and analyzing the vaginal swab sample profile produced in the aforementioned step. In some embodiments, the metabolite biomarkers are expressed in grade ½ endometrioid endometrial cancer (EEC).

In some embodiments, the methods described herein, including the aforementioned methods, are configured to predict the risk of endometrial hyperplasia or cancer in women and may also facilitate the diagnosis of endometrial hyperplasia or cancer. For instance, these methods may be utilized to diagnose endometrial cancer, such as Type 1 endometrial cancer (EC).

In some embodiments, a profile of a cervicovaginal sample (e.g., a CVL sample or a vaginal swab sample) is produced by detecting at least five or more metabolite biomarkers. In some embodiments, a profile of a cervicovaginal sample (e.g., a CVL sample or a vaginal swab sample) is produced by detecting at least ten or more metabolite biomarkers. In some embodiments, a profile of a cervicovaginal sample (e.g., a CVL sample or a vaginal swab sample) is produced by detecting at least fifteen or more metabolite biomarkers. In some embodiments, a profile of a cervicovaginal sample (e.g., a CVL sample or a vaginal swab sample) is produced by detecting at least twenty or more metabolite biomarkers. In some embodiments, a profile of a cervicovaginal sample (e.g., a CVL sample or a vaginal swab sample) is produced by detecting at least twenty five or more metabolite biomarkers.

In some embodiments, a profile of a cervicovaginal sample (e.g., a CVL sample or a vaginal swab sample) is produced by detecting about 5-25 metabolite biomarkers. In some embodiments, a profile of a cervicovaginal sample (e.g., a CVL sample or a vaginal swab sample) is produced by detecting about 5-20 metabolite biomarkers. In some embodiments, a profile of a cervicovaginal sample (e.g., a CVL sample or a vaginal swab sample) is produced by detecting about 5-15 metabolite biomarkers. In some embodiments, a profile of a cervicovaginal sample (e.g., a CVL sample or a vaginal swab sample) is produced by detecting about 5-10 metabolite biomarkers. In some embodiments, a profile of a cervicovaginal sample (e.g., a CVL sample or a vaginal swab sample) is produced by detecting about 10-25 metabolite biomarkers. In some embodiments, a profile of a cervicovaginal sample (e.g., a CVL sample or a vaginal swab sample) is produced by detecting about 10-20 metabolite biomarkers. In some embodiments, a profile of a cervicovaginal sample (e.g., a CVL sample or a vaginal swab sample) is produced by detecting about 10-15 metabolite biomarkers. In some embodiments, a profile of a cervicovaginal sample (e.g., a CVL sample or a vaginal swab sample) is produced by detecting about 15-25 metabolite biomarkers. In some embodiments, a profile of a cervicovaginal sample (e.g., a CVL sample or a vaginal swab sample) is produced by detecting about 15-20 metabolite biomarkers.

The present invention may also feature a non-invasive method of diagnosing endometrial cancer (EC) in a subject in need thereof. In some embodiments, the method comprises determining the subject's levels of five or more metabolites biomarkers: a) obtaining a biological sample (e.g., a cervicovaginal sample) from the patient; and b) measuring the levels of five or more metabolite biomarkers in the sample obtained. In some embodiments, the method comprises determining the patient's levels of five or more metabolite biomarkers by: obtaining a cervicovaginal lavage (CVL) sample from the patient; and measuring the levels of five or more biomarkers in the sample obtained. The patient may be diagnosed with EC if at least five biomarkers are altered compared to a control profile. In other embodiments, the method comprises determining the patient's levels of five or more metabolite biomarkers by: obtaining a vaginal swab sample from the patient; and measuring the levels of five or more biomarkers in the sample obtained. The patient may be diagnosed with EC if at least five biomarkers are altered compared to a control profile.

In some embodiments, the present invention features a non-invasive method of diagnosing endometrial cancer (EC) in a subject in need thereof. In some embodiments, the method comprises determining the subject's levels of five or more metabolite biomarkers and diagnosing the patient with EC if the levels of at least five biomarkers are altered compared to a control profile. In some embodiments, determining the subject's levels of five or more metabolite biomarkers comprises obtaining a cervicovaginal lavage (CVL) sample from the subject; and measuring the levels of at least five or more metabolite biomarkers in the sample obtained; wherein the metabolite biomarkers are selected from one or a combination of: 6-oxopiperidine-2-carboxylate, GPEA, GPC, guanine, cytosine, glycerophosphoserine, X-19913, X-24724, lyxonate, prolylglycine, glycerophosphoglycerol, or N-acetylserine. In certain embodiments, the subject is diagnosed with EC when 6-oxopiperidine-2-carboxylate, GPEA, GPC, guanine, cytosine, glycerophosphoserine, X-19913, X-24724, lyxonate, prolylglycine, glycerophosphoglycerol, or N-acetylserine are altered compared to a control profile.

In some embodiments, the present invention features a non-invasive method of diagnosing endometrial cancer (EC) in a subject in need thereof. In some embodiments, the method comprises determining the patient's levels of five or more metabolite biomarkers and diagnosing the subject with EC if the levels of at least five biomarkers are altered compared to a control profile. In some embodiments, determining the subject's levels of five or more metabolite biomarkers comprises obtaining a vaginal swab sample from the patient; and measuring the levels of at least five or more metabolite biomarkers in the sample obtained; wherein the metabolite biomarkers are selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP). In some embodiments, the a subject is diagnosed with EC with cancer when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are downregulated and gamma-glutamylglutamine, 1-stearoyl-GPI (18:0), or cytidine 5′-monophosphate (5′-CMP) are upregulated.

The present invention features a method of treating endometrial cancer (EC) in a patient in need thereof. In some embodiments, the method comprises diagnosing endometrial cancer (EC) in the patient as described herein. For example, the method of diagnosing EC may comprise obtaining a biological sample (e.g, a cervicovaginal sample) from the patient, measuring the levels of five or more metabolites biomarkers in the sample obtained, and diagnosing the patient with EC if the levels of at least five metabolite biomarkers are altered (e.g., increased) compared to a control profile. In some embodiments, the method of diagnosing EC may comprise obtaining a cervicovaginal lavage (CVL) sample from the patient, measuring the levels of five or more metabolites biomarkers in the sample obtained, and diagnosing the patient with EC if the levels of at least five biomarkers are altered (e.g., increased) compared to a control profile. In other embodiments, the method of diagnosing EC may comprise obtaining a vaginal swab sample from the patient, measuring the levels of five or more metabolites biomarkers in the sample obtained, and diagnosing the patient with EC if the levels of at least five biomarkers are altered (e.g., increased) compared to a control profile. A therapeutic amount of a treatment is administered to the patient if the patient is diagnosed with endometrial cancer (EC).

In some embodiments, the present invention features a method of treating endometrial cancer (EC) in a patient in need thereof. The method may comprise diagnosing EC in the patient as described herein and administering a therapeutic amount of a treatment to the patient if the patient is diagnosed with EC. In some embodiments, a patient may be diagnosed with EC by obtaining a cervicovaginal lavage (CVL) sample from the patient and measuring the levels of at least five or more metabolite biomarkers in the sample obtained in (i); wherein the metabolite biomarkers are selected from one or a combination of: 6-oxopiperidine-2-carboxylate, GPEA, GPC, guanine, cytosine, glycerophosphoserine, X-19913, X-24724, lyxonate, prolylglycine, glycerophosphoglycerol, or N-acetylserine; and diagnosing the patient with EC if the levels of at least five biomarkers in the profile obtained are altered compared to a control profile.

In some embodiments, the present invention features a method of treating endometrial cancer (EC) in a patient in need thereof. The method may comprise diagnosing EC in the patient as described herein and administering a therapeutic amount of a treatment to the patient if the patient is diagnosed with EC. In some embodiments, a patient may be diagnosed with EC by obtaining a vaginal swab sample from the patient, measuring the levels of at least five or more metabolite biomarkers in the sample obtained in (i); wherein the metabolite biomarkers are selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP); and diagnosing the patient with EC if the levels of at least five biomarkers are altered compared to a control profile.

In some embodiments, the aforementioned methods comprise measuring at least five or more metabolite biomarkers in a sample (e.g., cervicovaginal sample; e.g., a CVL sample or a vaginal swab sample). In some embodiments, the aforementioned methods comprise measuring at least ten or more metabolite biomarkers in a sample (e.g., cervicovaginal sample; e.g., a CVL sample or a vaginal swab sample). In some embodiments, the aforementioned methods comprise measuring at least fifteen or more metabolite biomarkers in a sample (e.g., cervicovaginal sample; e.g., a CVL sample or a vaginal swab sample). In some embodiments, the aforementioned methods comprise measuring at least twenty or more metabolite biomarkers in a sample (e.g. cervicovaginal sample; e.g., a CVL sample or a vaginal swab sample). In some embodiments, the aforementioned methods comprise measuring at least twenty five or more metabolite biomarkers in a sample (e.g., cervicovaginal sample; e.g., a CVL sample or a vaginal swab sample).

In some embodiments, the aforementioned methods comprise measuring about 5-25 metabolite biomarkers in a cervicovaginal sample (e.g., a CVL sample or a vaginal swab sample). In some embodiments, the aforementioned methods comprise measuring about 5-20 metabolite biomarkers in a cervicovaginal sample (e.g., a CVL sample or a vaginal swab sample). In some embodiments, the aforementioned methods comprise measuring about 5-15 metabolite biomarkers in a cervicovaginal sample (e.g., a CVL sample or a vaginal swab sample). In some embodiments, the aforementioned methods comprise measuring about 5-10 metabolite biomarkers in a cervicovaginal sample (e.g., a CVL sample or a vaginal swab sample). In some embodiments, the aforementioned methods comprise measuring about 10-25 metabolite biomarkers in a cervicovaginal sample (e.g., a CVL sample or a vaginal swab sample). In some embodiments, the aforementioned methods comprise measuring about 10-20 metabolite biomarkers in a cervicovaginal sample (e.g., a CVL sample or a vaginal swab sample). In some embodiments, the aforementioned methods comprise measuring about 10-15 metabolite biomarkers in a cervicovaginal sample (e.g., a CVL sample or a vaginal swab sample). In some embodiments, the aforementioned methods comprise measuring about 15-25 metabolite biomarkers in a cervicovaginal sample (e.g., a CVL sample or a vaginal swab sample). In some embodiments, the aforementioned methods comprise measuring about 15-20 metabolite biomarkers in a cervicovaginal sample (e.g., a CVL sample or a vaginal swab sample).

In some embodiments, a patient is diagnosed with EC if the levels of at least five or more metabolite biomarkers are altered compared to a control profile. In some embodiments, a patient is diagnosed with EC if the levels of at least ten or more metabolite biomarkers are altered compared to a control profile. In some embodiments, a patient is diagnosed with EC if the levels of at least fifteen or more metabolite biomarkers are altered compared to a control profile. In some embodiments, a patient is diagnosed with EC if the levels of at least twenty or more metabolite biomarkers are altered compared to a control profile. In some embodiments, a patient is diagnosed with EC if the levels of at least twenty-five or more metabolite biomarkers are altered compared to a control profile.

In some embodiments, a patient is diagnosed with EC if the levels of about 5-25 metabolite biomarkers are altered compared to a control profile. In some embodiments, a patient is diagnosed with EC if the levels of about 5-20 metabolite biomarkers are altered compared to a control profile. In some embodiments, a patient is diagnosed with EC if the levels of about 5-15 metabolite biomarkers are altered compared to a control profile. In some embodiments, a patient is diagnosed with EC if the levels of about 5-10 metabolite biomarkers are altered compared to a control profile. In some embodiments, a patient is diagnosed with EC if the levels of about 10-25 metabolite biomarkers are altered compared to a control profile. In some embodiments, a patient is diagnosed with EC if the levels of about 10-20 metabolite biomarkers are altered compared to a control profile. In some embodiments, a patient is diagnosed with EC if the levels of about 10-15 metabolite biomarkers are altered compared to a control profile. In some embodiments, a patient is diagnosed with EC if the levels of about 12-25 metabolite biomarkers are altered compared to a control profile. In some embodiments, a patient is diagnosed with EC if the levels of about 15-20 metabolite biomarkers are altered compared to a control profile.

The present may also feature a method of monitoring treatment for endometrial cancer (EC) in a subject in need thereof. In some embodiments, the method comprises obtaining a first biological sample from the subject and producing a baseline profile of the biological sample collected by detecting at least five or more metabolites biomarkers. The treatment for EC is then administered to the subject. The method may further comprise obtaining a second biological sample from the subject and producing a second profile of the biological sample collected by detecting at least five or more metabolites biomarkers. The baseline profile of the biological sample produced may then be compared to the second profile of the biological sample produced. In some embodiments, the treatment is effective if the levels of at least five biomarkers are altered from the baseline profile as compared to the second profile.

In other embodiments, the method comprises obtaining a first cervicovaginal lavage (CVL) sample from the subject and producing a baseline profile of the CVL sample collected by detecting at least five or more metabolite biomarkers. For example, the baseline profile may be produced by detecting five or more metabolite biomarkers are selected from one or a combination of: 6-oxopiperidine-2-carboxylate, GPEA, GPC, guanine, cytosine, glycerophosphoserine, X-19913, X-24724, lyxonate, prolylglycine, glycerophosphoglycerol, or N-acetylserine. In some embodiments, the method comprises administering the treatment for EC to the subject. The method may further comprise obtaining a second CVL sample from the subject and producing a second profile of the CVL sample collected by detecting at least five or more metabolites biomarkers. For example, the second profile may be produced by detecting five or more metabolite biomarkers are selected from one or a combination of: 6-oxopiperidine-2-carboxylate, GPEA, GPC, guanine, cytosine, glycerophosphoserine, X-19913, X-24724, lyxonate, prolylglycine, glycerophosphoglycerol, or N-acetylserine. In some embodiments, the method comprises comparing the baseline profile of the CVL sample to the second profile of the CVL sample. In some embodiments, the treatment is effective if the levels of at least five biomarkers are altered from the baseline profile as compared to the second profile.

In other embodiments, the method comprises obtaining a first vaginal swab sample from the subject and producing a baseline profile of the vaginal swab sample collected by detecting at least five or more metabolite biomarkers. For example, the baseline profile may be produced by detecting five or more metabolite biomarkers selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP). In some embodiments, the method comprises administering the treatment for EC to the subject. The method may further comprise obtaining a second vaginal swab sample from the subject and producing a second profile of the vaginal swab sample collected by detecting at least five or more metabolites biomarkers. For example, the second profile may be produced by detecting five or more metabolite biomarkers selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP). In some embodiments, the method comprises comparing the baseline profile of the vaginal swab sample to the second profile of the vaginal swab sample. In some embodiments, the treatment is effective if the levels of at least five biomarkers are altered from the baseline profile as compared to the second profile. In some embodiments, the treatment is effective when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are upregulated and gamma-glutamylglutamine, 1-stearoyl-GPI (18:0), or cytidine 5′-monophosphate (5′-CMP) are downregulated compared to a control profile.

In some embodiments, the treatment is effective if the levels of at least five biomarkers from the baseline profile are altered as compared to the second profile. In some embodiments, the treatment is effective if the levels of at least ten biomarkers from the baseline profile are altered as compared to the second profile. In some embodiments, the treatment is effective if the levels of at least fifteen biomarkers from the baseline profile are altered as compared to the second profile. In some embodiments, the treatment is effective if the levels of at least twenty biomarkers from the baseline profile are altered as compared to the second profile. In some embodiments, the treatment is effective if the levels of at least twenty-five biomarkers from the baseline profile are altered as compared to the second profile.

In some embodiments, the treatment is effective if the levels of about 5-25 biomarkers from the baseline profile are altered as compared to the second profile. In some embodiments, the treatment is effective if the levels of about 5-20 biomarkers from the baseline profile are altered as compared to the second profile. In some embodiments, the treatment is effective if the levels of about 5-15 biomarkers from the baseline profile are altered as compared to the second profile. In some embodiments, the treatment is effective if the levels of about 5-20 biomarkers from the baseline profile are altered as compared to the second profile. In some embodiments, the treatment is effective if the levels of about 10-25 biomarkers from the baseline profile are altered as compared to the second profile. In some embodiments, the treatment is effective if the levels of about 10-20 biomarkers from the baseline profile are altered as compared to the second profile. In some embodiments, the treatment is effective if the levels of about 10-15 biomarkers from the baseline profile are altered as compared to the second profile. In some embodiments, the treatment is effective if the levels of about 15-25 biomarkers from the baseline profile are altered as compared to the second profile. In some embodiments, the treatment is effective if the levels of about 15-20 biomarkers from the baseline profile are altered as compared to the second profile.

The present invention may also feature an in vitro method of diagnosing endometrial cancer (EC) in a subject in need thereof. The method may comprise producing a profile from a biological sample obtained from a subject by detecting at least five or more metabolite biomarkers and analyzing the biological sample profile produced. In some embodiments, the subject is diagnosed with EC if the levels of at least five biomarkers are altered compared to a control profile. The present invention may also feature methods of treating endometrial cancer (EC) in a subject in need thereof, where if a subject is diagnosed with EC, then an EC treatment is administered to the subject.

In some embodiments, the present invention may also feature an in vitro method of diagnosing endometrial cancer (EC) in a subject in need thereof. The method may comprise producing a profile from a CVL sample having been obtained from a subject by detecting at least five or more metabolite biomarkers selected from one or a combination of. 6-oxopiperidine-2-carboxylate, GPEA, GPC, guanine, cytosine, glycerophosphoserine, X-19913, X-24724, lyxonate, prolylglycine, glycerophosphoglycerol, or N-acetylserine, and diagnosing the patient with EC if the levels of at least five biomarkers are altered compared to a control profile. In some embodiments, the subject is diagnosed with EC if the levels of at least five biomarkers are altered compared to a control profile. The present invention may also feature methods of treating endometrial cancer (EC) in a subject in need thereof, where if a subject is diagnosed with EC, then an EC treatment is administered to the subject. In some embodiments, the the method diagnoses endometrial cancer (EC) in the subject, wherein a subject is diagnosed with EC with cancer when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are downregulated and gamma-glutamylglutamine, 1-stearoyl-GPI (18:0), or cytidine 5′-monophosphate (5′-CMP) are upregulated compared to a control profile.

In some embodiments, the present invention may also feature an in vitro method of diagnosing endometrial cancer (EC) in a subject in need thereof. The method may comprise producing a profile from a vaginal swab sample obtained from a subject by detecting at least five or more metabolite biomarkers selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP)and diagnosing the patient with EC if the levels of at least five biomarkers are altered compared to a control profile. In some embodiments, the subject is diagnosed with EC if the levels of at least five biomarkers are altered compared to a control profile. The present invention may also feature methods of treating endometrial cancer (EC) in a subject in need thereof, where if a subject is diagnosed with EC, then an EC treatment is administered to the subject. In some embodiments, the a subject is diagnosed with EC with cancer when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are downregulated and gamma-glutamylglutamine, 1-stearoyl-GPI (18:0), or cytidine 5′-monophosphate (5′-CMP) are upregulated.

In some embodiments, aforementioned methods described herein may comprise measuring five or more metabolic metabolites and/or characterizing endometrial tumor characteristics. In some embodiments, aforementioned methods described herein may further characterize endometrial tumor characteristics, such as tumor size, myometrial invasion, mismatch repair (MMR) status, histological grade, age, or a combination thereof.

In some embodiments, metabolite biomarkers that may indicate tumor size in a CVL sample may include but are not limited to dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, and cytosine. In some embodiments, dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) are enriched (e.g., upregulated) in tumors greater than or equal to 2 cm. In other embodiments, CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, cytosine are depleted (e.g., down-regulated) in tumors greater than or equal to 2 cm.

In other embodiments, metabolite biomarkers that may indicate tumor size in a CVL sample may include but are not limited (3′-5′)-adenylylcytidine, (3′-5′)-adenylyluridine, (3′-5′)-cytidylyluridine, (3′-5′)-guanylylcytidine, (3′-5′)-guanylyluridine, 1-(1-enyl-palmitoyl)-2-linoleoyl-GPC (P-16:0/18:2), 1-(1-enyl-palmitoyl)-2-oleoyl-GPC (P-16:0/18:1), 1-(1-enyl-palmitoyl)-2-oleoyl-GPE (P-16:0/18:1), 18:0-18:1 PS, 1-linoleoylglycerol (18:2), 1-methylguanidine, 1-oleoyl-GPC (18:1), 1-oleoyl-GPE (18:1), 1-palmitoyl-2-stearoyl-GPC (16:0/18:0), 1-palmitoyl-GPC (16:0), 1-palmitoyl-GPE (16:0), 1-stearoyl-2-oleoyl-GPS (18:0/18:1), 1-stearoyl-GPI (18:0), 2,3-diphosphoglycerate, 2′-deoxyuridine, 2-palmitoyl-GPC* (16:0), 3-hydroxypalmitoylcarnitine, 3-methyl-2-oxovalerate, 4-hydroxyphenylacetylglutamine, argininate, behenoyl dihydrosphingomyelin (d18:0/22:0), BHBA, bilirubin degradation product, C17H18N2O4 (2), biliverdin, C14 Cer, C16 Cer, C16 LacCer, C16DH Cer, C17 Cer, C18 (Plasm)-18:1 PE, C18 Plasm Ipe, C24:1 LacCer, carnosine, CDP-choline, CDP-ethanolamine, cysteine, cytidine diphosphate, cytosine, dihomolinoleate (20:2n6), dihomolinolenate (20:3n3 or 3n6), dihydroorotate, eicosenoate (20:1n9 or 1n11), erucate (22:1n9), glutathione, reduced (GSH), glycerophosphoglycerol, GPE (18:0/18:1), GPE (P-16:0/20:4), histidylalanine, homocysteine, Isobar: hexose diphosphates, MAG (20:3), Mha acid, myristoylcarnitine (C14), N6-methyladenosine, N-acetylaspartate (NAA), N-acetylcysteine, N-acetyltaurine, NAD+, N-palmitoyl-sphingadienine (d18:2/16:0), N-stearoyl-sphinganine (d18:0/18:0), N-stearoyl-sphingosine (d18:1/18:0), palmitoleoylcarnitine (C16:1), palmitoylcarnitine (C16), PC (14:0/16:0), PC (P-16:0/16:0), PC (P-16:0/20:4), sphingomyelin (d18:2/24:1, d18:1/24:2), tyrosylglycine, val-val-ala, or valylglutamine.

In some embodiments, metabolite biomarkers that may indicate myometrial invasion in a CVL sample may include but are not limited to ceramide (d18:1/14:0, d16:1/16:0)*, (3′-5′)-cytidylyluridine*, N-stearoyl-sphingosine (d18:1/18:0)*, N-palmitoyl-sphinganine (d18:0/16:0), N-palmitoyl-sphingosine (d18:1/16:0), 1-(1-enyl-palmitoyl)-2-palmitoyl-GPC (P-16:0/16:0)*, alpha-hydroxyisocaproate, CMP, 2′-AMP, AMP, argininate*, 2,3-diphosphoglycerate, cyclic adenosine diphosphate-ribose, histamine, and tryptamine. In some embodiments, ceramide (d18:1/14:0, d16:1/16:0)*, (3′-5′)-cytidylyluridine*, N-stearoyl-sphingosine (d18:1/18:0)*, N-palmitoyl-sphinganine (d18:0/16:0), N-palmitoyl-sphingosine (d18:1/16:0), and 1-(1-enyl-palmitoyl)-2-palmitoyl-GPC (P-16:0/16:0)* are enriched (e.g., upregulated) in the absence of myometrial invasion. In some embodiments, alpha-hydroxyisocaproate, CMP, 2′-AMP, AMP, argininate*, 2,3-diphosphoglycerate, cyclic adenosine diphosphate-ribose, histamine, and tryptamine are depleted (e.g., down-regulated) in the absence of myometrial invasion.

In some embodiments, metabolite biomarkers that may indicate myometrial invasion in a CVL sample may include but are not limited to (3′-5′)-cytidylyluridine, 2,3-diphosphoglycerate, 2′-AMP, alpha-hydroxyisocaproate, AMP, argininate, C14 Cer, C16 Cer, C16DH Cer, CMP, cyclic adenosine diphosphate-ribose, histamine, N-stearoyl-sphingosine (d18:1/18:0), PC (P-16:0/16:0), or tryptamine.

In some embodiments, metabolite biomarkers that may indicate MMR status in a CVL sample may include but are not limited to decanoylcarnitine (C10), octanoylcarnitine (C8), laurylcarnitine (C12), glutarate (C5-DC), S-adenosylmethionine (SAM), butyrylcarnitine (C4), lyxonate, 6-oxopiperidine-2-carboxylate, adenosine, guanine, and sarcosine. In some embodiments, decanoylcarnitine (C10), octanoylcarnitine (C8), and laurylcarnitine (C12) are enriched (e.g., upregulated) in MMR-proficient tumors. In some embodiments, glutarate (C5-DC), S-adenosylmethionine (SAM), butyrylcarnitine (C4), lyxonate, 6-oxopiperidine-2-carboxylate, adenosine, guanine, and sarcosine are depleted (e.g., down-regulated) in MMR deficient tumors.

In other embodiments, metabolite biomarkers that may indicate MMR status in a CVL sample may include but are not limited to 6-oxopiperidine-2-carboxylate, adenosine, butyrylcarnitine (C4), decanoylcarnitine (C10), glutarate (C5-DC), guanine, laurylcarnitine (C12), lyxonate, octanoylcarnitine (C8), SAM, or sarcosine.

In some embodiments, metabolite biomarkers that may indicate histological grade in a CVL sample may include but are not limited to 1-oleoyl-GPS (18:1), pregnen-diol disulfate, dehydroepiandrosterone sulfate (DHEA-S), kynurenine, 1-(1-enyl-palmitoyl)-2-arachidonoyl-GPC (P-16:0/20:4), pregnenetriol disulfate, 1-stearoyl-2-docosahexaenoyl-GPC (18:0/22:6), 1,2-dilinoleoyl-GPC (18:2/18:2), 1-palmitoyl-2-palmitoleoyl-GPC (16:0/16:1), 1-linoleoyl-2-arachidonoyl-GPC (18:2/20:4n6), androsterone sulfate, 1-palmitoyl-2-arachidonoyl-GPC (16:0/20:4n6), androstenediol (3beta,17beta) disulfate (2), 1-stearoyl-2-linoleoyl-GPC (18:0/18:2), 1-palmitoyl-2-docosahexaenoyl-GPC (16:0/22:6), 1-stearoyl-2-arachidonoyl-GPC (18:0/20:4), N-methylhydroxyproline, 3-hydroxybutyrate (BHBA), sphingomyelin (d18:2/16:0, d18:1/16:1), 1-palmitoyl-2-dihomo-linolenoyl-GPC (16:0/20:3n3 or 6), 1-palmitoyl-2-linoleoyl-GPC (16:0/18:2), sphingomyelin (d18:1/20:1, d18:2/20:0), sphingomyelin (d18:1/18:1, d18:2/18:0), 1-(1-enyl-palmitoyl)-2-linoleoyl-GPC (P-16:0/18:2), 1-oleoyl-2-linoleoyl-GPC (18:1/18:2), 2-methylbutyrylcarnitine (C5), and homocysteine. In some embodiments, 1-oleoyl-GPS (18:1), pregnen-diol disulfate, dehydroepiandrosterone sulfate (DHEA-S), kynurenine, 1-(1-enyl-palmitoyl)-2-arachidonoyl-GPC (P-16:0/20:4), pregnenetriol disulfate, 1-stearoyl-2-docosahexaenoyl-GPC (18:0/22:6), 1,2-dilinoleoyl-GPC (18:2/18:2), 1-palmitoyl-2-palmitoleoyl-GPC (16:0/16:1), 1-linoleoyl-2-arachidonoyl-GPC (18:2/20:4n6), androsterone sulfate, 1-palmitoyl-2-arachidonoyl-GPC (16:0/20:4n6), androstenediol (3beta,17beta) disulfate (2), 1-stearoyl-2-linoleoyl-GPC (18:0/18:2), 1-palmitoyl-2-docosahexaenoyl-GPC (16:0/22:6), 1-stearoyl-2-arachidonoyl-GPC (18:0/20:4), N-methylhydroxyproline, 3-hydroxybutyrate (BHBA), sphingomyelin (d18:2/16:0, d18:1/16:1), 1-palmitoyl-2-dihomo-linolenoyl-GPC (16:0/20:3n3 or 6), 1-palmitoyl-2-linoleoyl-GPC (16:0/18:2), sphingomyelin (d18:1/20:1, d18:2/20:0), sphingomyelin (d18:1/18:1, d18:2/18:0), 1-(1-enyl-palmitoyl)-2-linoleoyl-GPC (P-16:0/18:2), and 1-oleoyl-2-linoleoyl-GPC (18:1/18:2) are enriched (e.g., upregulated) in ½ EEC. In some embodiments, 2-methylbutyrylcarnitine (C5) and homocysteine are depleted (e.g., down-regulated) in ½ EEC.

In some embodiments, metabolite biomarkers that may indicate histological grade in a CVL sample may include but are not limited to 1-(1-enyl-palmitoyl)-2-linoleoyl-GPC (P-16:0/18:2), 1,2-dilinoleoyl-GPC (18:2/18:2), 1-linoleoyl-2-arachidonoyl-GPC (18:2/20:4n6), 1-oleoyl-2-linoleoyl-GPC (18:1/18:2), 1-oleoyl-GPS (18:1), 1-palmitoyl-2-docosahexaenoyl-GPC (16:0/22:6), 1-palmitoyl-2-linoleoyl-GPC (16:0/18:2), 1-stearoyl-2-docosahexaenoyl-GPC (18:0/22:6), 1-stearoyl-2-linoleoyl-GPC (18:0/18:2), 2-methylbutyrylcarnitine (C5), androstenediol (3beta, 17beta) disulfate (2), androsterone sulfate, BHBA, dehydroepiandrosterone sulfate (DHEA-S), GPC (16:0/20:3), homocysteine, kynurenine, N-methylhydroxyproline, PC (16:0/16:1), PC (16:0/20:4), PC (18:0/20:4n6), PC (P-16:0/20:4), pregnen-diol disulfate, pregnenetriol disulfate, SM (d18:1/16:0, d18:1/16:1(92)), sphingomyelin (d18:1/18:1, d18:2/18:0), sphingomyelin (d18:1/20:1, d18:2/20:0), or sphingomyelin (d18:2/24:1, d18:1/24:2).

In some embodiments, metabolite biomarkers that may indicate age in a CVL sample may include but are not limited to N6-methyllysine, nicotinate ribonucleoside, or a combination thereof

In some embodiments, metabolite biomarkers that may indicate tumor size in a vaginal swab sample may include but are not limited 3,7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991. In some embodiments, the aforementioned metabolite biomarkers are depleted (e.g., down-regulated) in tumors greater than or equal to 2 cm.

In some embodiments, metabolite biomarkers that may indicate myometrial invasion in a vaginal swab sample may include but are not limited dihomo-linolenate (20:3n3 or n6), 1-(1-enyl-oleoyl)-GPE (P-18:1)*, 1-(1-enyl-palmitoyl)-GPE (P-16:0)*, 1-stearoyl-GPE (18:0), 1-(1-enyl-palmitoyl)-2-docosahexaenoyl-GPE (P-16:0/22:6)*, 1,2-dilinoleoyl-GPC (18:2/18:2), 1-(1-enyl-palmitoyl)-2-linoleoyl-GPC (P-16:0/18:2)*, 1-stearoyl-GPC (18:0), alpha-tocopherol, 1,2-dipalmitoyl-GPC (16:0/16:0), 1-(1-enyl-palmitoyl)-2-arachidonoyl-GPC (P-16:0/20:4)*, myristoylcarnitine (C14), 1-(1-enyl-palmitoyl)-2-oleoyl-GPC (P-16:0/18:1)*, X-17348, vitamin D3 sulfate, ondansetron, and N-acetylhistamine. In some embodiments, dihomo-linolenate (20:3n3 or n6), 1-(1-enyl-oleoyl)-GPE (P-18:1)*, 1-(1-enyl-palmitoyl)-GPE (P-16:0)*, 1-stearoyl-GPE (18:0), 1-(1-enyl-palmitoyl)-2-docosahexaenoyl-GPE (P-16:0/22:6)*, 1,2-dilinoleoyl-GPC (18:2/18:2), 1-(1-enyl-palmitoyl)-2-linoleoyl-GPC (P-16:0/18:2)*, 1-stearoyl-GPC (18:0), alpha-tocopherol, 1,2-dipalmitoyl-GPC (16:0/16:0), 1-(1-enyl-palmitoyl)-2-arachidonoyl-GPC (P-16:0/20:4)*, myristoylcarnitine (C14), 1-(1-enyl-palmitoyl)-2-oleoyl-GPC (P-16:0/18:1)* are enriched (e.g., upregulated) in the absence of myometrial invasion. In some embodiments, X-17348, vitamin D3 sulfate, ondansetron, and N-acetylhistamine are depleted (e.g., down-regulated) in the absence of myometrial invasion.

In some embodiments, metabolite biomarkers that may indicate MMR status in a vaginal swab sample may include but are not limited lidocaine, 3-methylglutarate/2-methylglutarate, sphingomyelin (d17:1/14:0, d16:1/15:0)*, 4-cholesten-3-one, adenosine, margaroylcarnitine (C17)*, oleoylcarnitine (C18:1), 2-hydroxyadipate, 2,3-dihydroxyisovalerate, 2-isopropylmalate, and sarcosine. In some embodiments, the aforementioned metabolite biomarkers are depleted (e.g., down-regulated) in MMR proficient cancer.

In some embodiments, metabolite biomarkers that may indicate histological grade in a CVL sample may include but are not limited to X-11308 or 4-hydroxyglutamate. In some embodiments, are X-11308 is enriched (e.g., upregulated) in ½ EEC and 4-hydroxyglutamate is depleted (e.g., down-regulated) in ½ EEC.

The present invention may also feature a non-invasive method of determining the size of a tumor in a subject with endometrial cancer (EC). The method may comprise determining the patient's levels of five or more metabolites biomarkers by obtaining a biological sample (e.g., a CVL sample or a vaginal swab) from the patient and measuring the levels of five or more biomarkers in the sample (e.g., the CVL sample) obtained. In some embodiments, the five or more biomarkers are selected from a group comprising 2-hydroxybutyrate, 2′-deoxyuridine, 2-methyl-2-oxybutyrate, 5-methyluridine, 5,6,-dihydrothymine, adenosine, AMP, carnosine, cytidine diphosphate, FA(20:2n6), FA(20:3n6), hexose diphosphates, N-acetylglucosamine, N-acetyltaurine, C16DH Cer, C16 Cer, SAM, uridine or a combination thereof. The size of the tumor is greater than 2 cm if the levels of two or more biomarkers are altered compared to a predetermined threshold

In some embodiments, the present invention features a non-invasive method of determining a size of a tumor in a subject with endometrial cancer (EC). The method may comprise determining the patient's levels of five or more metabolites biomarkers by obtaining a cervicovaginal lavage (CVL) sample from the patien; and measuring the levels of five or more biomarkers in the sample obtained. In some embodiments, the five or more biomarkers comprise dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, and cytosine. The size of the tumor is greater than 2 cm if the levels of five or more biomarkers are altered compared to a predetermined threshold. In certain embodiments, dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) are upregulated in tumors greater than or equal to 2 cm and CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, cytosine are down-regulated in tumors greater than or equal to 2 cm.

In other embodiments, the present invention features a non-invasive method of determining a size of a tumor in a subject with endometrial cancer (EC). The method may comprise determining the patient's levels of five or more metabolites biomarkers by obtaining a vaginal swab sample from the patient, and measuring the levels of five or more biomarkers in the sample obtained. In some embodimetns, the five or more biomarkers comprise 7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991. The size of the tumor is greater than 2 cm if the levels of five or more biomarkers are altered compared to a predetermined threshold. In certain embodiments, 7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991 are down-regulated in tumors greater than or equal to 2 cm.

In some embodiments, the size of the tumor is greater than 2 cm if the levels of at least five or more biomarkers are altered compared to a predetermined threshold. In some embodiments, the size of the tumor is greater than 2 cm if the levels of at least ten or more biomarkers are altered compared to a predetermined threshold. In some embodiments, the size of the tumor is greater than 2 cm if the levels of at least fifteen or more biomarkers are altered compared to a predetermined threshold. In some embodiments, the size of the tumor is greater than 2 cm if the levels of at least twenty or more biomarkers are altered compared to a predetermined threshold. In some embodiments, the size of the tumor is greater than 2 cm if the levels of at least twenty-five or more biomarkers are altered compared to a predetermined threshold.

In some embodiments, the size of the tumor is greater than 2 cm if the levels of about 5-25 biomarkers are altered compared to a predetermined threshold. In some embodiments, the size of the tumor is greater than 2 cm if the levels of about 5-20 biomarkers are altered compared to a predetermined threshold. In some embodiments, the size of the tumor is greater than 2 cm if the levels of about 5-15 biomarkers are altered compared to a predetermined threshold. In some embodiments, the size of the tumor is greater than 2 cm if the levels of about 5-10 biomarkers are altered compared to a predetermined threshold. In some embodiments, the size of the tumor is greater than 2 cm if the levels of about 10-25 biomarkers are altered compared to a predetermined threshold. In some embodiments, the size of the tumor is greater than 2 cm if the levels of about 10-20 biomarkers are altered compared to a predetermined threshold. In some embodiments, the size of the tumor is greater than 2 cm if the levels of about 10-15 biomarkers are altered compared to a predetermined threshold. In some embodiments, the size of the tumor is greater than 2 cm if the levels of about 15-25 biomarkers are altered compared to a predetermined threshold. In some embodiments, the size of the tumor is greater than 2 cm if the levels of about 15-20 biomarkers are altered compared to a predetermined threshold.

The present invention may further feature a non-invasive method of determining a prognosis of endometrial cancer (EC) in a subject in need thereof. In some embodiments, the method comprises characterizing endometrial tumor characteristics comprising tumor size, myometrial invasion, mismatch repair (MMR) status, histological grade, age, or a combination thereof and determining the prognosis of the patient. In some embodiments, the endometrial tumor characteristics may be determined by determining the patient's levels of five or more metabolites biomarkers by obtaining a biological sample from the patient; and measuring the levels of two or more biomarkers in the sample obtained. In some embodiments, a tumor size larger than 2 cm, presence of myometrial invasion, MMR proficient, and grade 3 is indicative of a poor prognosis and a tumor size smaller than 2 cm, no myometrial invasion, MMR deficient and grade ½ is indicative of a good prognosis. In some embodiments, the biological sample comprises a cervicovaginal lavage (CVL) sample, a urine sample, a vaginal swab, or a cervicovaginal secretion; wherein the cervicovaginal secretion is collected via a self collected lavage or a menstrual cup.

In some embodiments, the method comprises characterizing endometrial tumor characteristics comprising tumor size, myometrial invasion, mismatch repair (MMR) status, histological grade, age, or a combination thereof and determining the prognosis of the patient. In some embodiments, the endometrial tumor characteristics may be determined by determining the patient's levels of five or more metabolites biomarkers by obtaining a CVL sample from the patient; and measuring the levels of two or more biomarkers in the sample obtained. In some embodiments, a tumor size larger than 2 cm, presence of myometrial invasion, MMR proficient, and grade 3 is indicative of a poor prognosis and a tumor size smaller than 2 cm, no myometrial invasion, MMR deficient and grade ½ is indicative of a good prognosis. In some embodiments, the method comprises characterizing endometrial tumor characteristics comprising tumor size, myometrial invasion, mismatch repair (MMR) status, histological grade, age, or a combination thereof and determining the prognosis of the patient. In some embodiments, the endometrial tumor characteristics may be determined by determining the patient's levels of five or more metabolites biomarkers by obtaining a vaginal swab sample from the patient; and measuring the levels of two or more biomarkers in the sample obtained. In some embodiments, a tumor size larger than 2 cm, presence of myometrial invasion, MMR proficient, and grade 3 is indicative of a poor prognosis and a tumor size smaller than 2 cm, no myometrial invasion, MMR deficient and grade ½ is indicative of a good prognosis.

In some embodiments, the method comprises characterizing endometrial tumor characteristics comprising tumor size, myometrial invasion, mismatch repair (MMR) status, histological grade, age, or a combination thereof and determining the prognosis of the patient. In some embodiments, the endometrial tumor characteristics may be determined by determining the patient's levels of five or more metabolites biomarkers by obtaining a vaginal swab sample from the patient; and measuring the levels of two or more biomarkers in the sample obtained. In some embodiments, a tumor size larger than 2 cm, presence of myometrial invasion, MMR proficient, and grade 3 is indicative of a poor prognosis and a tumor size smaller than 2 cm, no myometrial invasion, MMR deficient and grade ½ is indicative of a good prognosis. In some embodiments, the method comprises characterizing endometrial tumor characteristics comprising tumor size, myometrial invasion, mismatch repair (MMR) status, histological grade, age, or a combination thereof and determining the prognosis of the patient. In some embodiments, the endometrial tumor characteristics may be determined by determining the patient's levels of five or more metabolites biomarkers by obtaining a vaginal swab sample from the patient; and measuring the levels of two or more biomarkers in the sample obtained. In some embodiments, a tumor size larger than 2 cm, presence of myometrial invasion, MMR proficient, and grade 3 is indicative of a poor prognosis and a tumor size smaller than 2 cm, no myometrial invasion, MMR deficient and grade ½ is indicative of a good prognosis.

Various methods may be used to produce a profile in accordance with the present invention. In some embodiments, a bioinformatic pipeline may be used to build and predict said profile.

### Example 1

The following is a non-limiting example of the present invention. It is to be understood that said example is not intended to limit the present invention in any way. Equivalents or substitutes are within the scope of the present invention.

Endometrial cancer (EC) was previously grouped into two major categories: type I (consisting of grade 1 and 2 endometrioid carcinoma (EEC)) and type II (composed of higher-grade EECs and other non-endometrioid subtypes). Now, due to the heterogeneity of EC, different subtypes have been proposed based on histology and genetic information, such as mutations in p53, mismatch repair (MMR) proteins, and POLE. Grade ½ EEC is more common, estrogen-driven, and has a better prognosis; Grade 3 EEC and other EC subtypes are less common, are not estrogen-driven, and have a poorer prognosis.

Several factors determine the prognosis of EC, including age, histological grade, tumor size, presence of lymphovascular or myometrial invasion, and MMR protein status. Regardless of the prognosis, the gold standard for EC treatment remains total hysterectomy, removal of ovaries and tubes, and either sentinel or complete pelvic lymphadenectomy; however, this procedure can be undesirable, especially for younger women wanting to preserve fertility and/or prevent early menopause. The present invention features novel diagnostic and prognostic biomarkers that are crucial for better risk stratification and improved treatment options for EC patients.

The present invention utilizes an untargeted global metabolomics platform in combination with cervicovaginal sampling from a cohort of well-characterized patients undergoing hysterectomy (n=192) to identify metabolomic profiles in women with EC. This foundational knowledge is essential for advancing pathophysiological understanding of the disease, as well as improving detection and risk stratification based on tumor progression and characteristics.

Study participants: Participants were recruited at three clinical sites in the Phoenix (AZ, USA) metropolitan area. A total of 192 women undergoing hysterectomy for benign or malignant indications were enrolled. Histopathology results from biopsy samples collected from surgery were used to stratify participants into four disease groups: benign conditions (n=108) (including adenomyosis, endometriosis, fibroids, and other benign implications as singular or comorbidities), endometrial hyperplasia (n=18), grade 1 or 2 endometrioid carcinoma (grade ½ EEC) (n=53), and other endometrial cancer (EC) subtypes (n=13) (including grade 3 EEC, serous carcinoma and other histological subtypes). A breakdown of the diagnosis of hyperplasia and endometrial cancer subtypes can be found in Table 1. Women of any race or ethnicity and ages 18 years or older were included. Patients were excluded based on factors such as menstruation, infectious diseases, lifestyle choices, and other conditions; detailed exclusion criteria are available in Table 2. The exclusion criteria were verified by physician's pelvic exam, medical records and/or self-reported data. Demographic, socioeconomic, and medical history data were collected from surveys and/or medical records.

Table 1 shows the breakdown of diagnoses of hyperplasia and endometrial cancer subtypes. Values are n (%). Benign diagnosis breakdown is not included as these participants had a mixture of both singular and co-occurring conditions such as adenomyosis, endometriosis, and fibroids.

| | Histological type | n (%) |
|---|---|---|
| Hyperplasia | Complex hyperplasia with atypia | 11 (61.1) |
| (n = 18) | Complex hyperplasia without atypia | 3 (16.7) |
| | Simple hyperplasia with atypia | 2 (11.1) |
| | Simple hyperplasia without atypia | 1 (5.6) |
| | Hyperplasia without cancer | 1 (5.6) |
| Endometrial | Endometrioid adenocarcinoma | 33 (50.0) |
| Cancer (n = 66) | Endometrioid carcinoma | 26 (39.4) |
| | Serous carcinoma | 4 (6.1) |
| | Other | 3 (4.5) |

Table 2 shows the exclusion criteria for participant recruitment into the study.

Exclusion Criteria

| Time frame in regard to | |
|---|---|
| hysterectomy | Condition |
| Currently | Menstruating |
| | Lactating |
| | On antifungals, antivirals, antibiotics |
| | or topical steroids |
| | Vaginal infection (bacterial vaginosis, |
| | candidiasis) |
| | Vulvar infection |
| | Urinary tract infection |
| Current or within previous | Sexually transmitted infection (chlamydia, |
| three weeks | gonorrhoea, trichomoniasis, genital herpes) |
| Within previous 2 hours | Smoking or consumed nicotine-containing |
| | products |
| Within previous 4 hours | Bath or swimming |
| Within previous 48 hours | Use of douching substances |
| | Vaginal medications |
| | Vaginal suppositories |
| | Feminine deodorant sprays, wipes, |
| | or lubricants |
| | Sexual intercourse |
| Within previous 72 hours | Use of depilatory treatments in the |
| | genital area |
| N/A | Any skin condition in the genital area |
| | interfering with the study |
| | Hepatitis |
| | Being HIV-positive |

Sample collection and processing: CVL and vaginal swab samples were collected by a surgeon in the operating room during standard-of-care hysterectomy procedure. Samples were obtained after induction of anesthesia and prior to vaginal preparation with antiseptic solution. CVLs were collected using a non-lubricated speculum and 10 ml of sterile 0.9% saline solution (Teknova, Hollister, CA). Samples were immediately placed on ice and frozen at −80° C. within an hour. Prior to downstream analyses, the samples were thawed on ice; centrifuged (700×g for 10 minutes at 4° C.); aliquoted to prevent multiple freeze-thaw cycles; and stored at −80° C.

Quantification of soluble metabolites: Soluble metabolites in CVL and vaginal swab samples were quantified using a global metabolomics platform at Metabolon, Inc (Durham, NC) as previously described. Briefly, the Metabolon's platform utilized a Waters ACQUITY ultra-performance liquid chromatography (UPLC) and a Thermo Scientific Q-exactive high resolution/accurate mass spectrometer interfaced with a heated electrospray ionization (HESI-II) source and Orbitrap mass analyzer operated at 35000 mass resolution. Compounds were identified using Metabolon's library of purified standards. Peaks were quantified using the area under the curve for relative intensity. The data were normalized by registering medians of each compound to equal one and normalizing each data point proportionately.

Metabolomic data analysis: MetaboAnalyst 5.0 was used to analyze and visualize metabolomic data. All data input into MetaboAnalyst were log₁₀-transformed and autoscaled (mean-centered and divided by the standard deviation of each variable).

Unsupervised hierarchical clustering analysis (HCA) was performed on metabolomic data to visualize metabolic profiles as heatmaps and show relationships between global metabolic profiles and disease groups: EC-All (grade ½ EEC and other EC subtypes), endometrial hyperplasia, and benign conditions. For sample clustering, the Pearson distance measure and the Ward linkage method were applied. HCA utilized the top 100 significant metabolites (q<0.05) based on an analysis of variance (ANOVA) with false discovery rate (FDR)-correction.

A two-sample T-test with FDR-correction (q<0.05) was performed to determine significant differences in metabolite levels between the disease groups. Fold change (FC) analysis was used to compare the absolute value of the change in the means of each metabolite between the two groups being investigated. The FC analysis utilizes data prior to data transformation and scaling. Data from FC analysis and T-test were combined to produce volcano plots depicting significantly up/downregulated metabolites (q<0.05 and FC>2) for comparison of two selected disease groups.

Enrichment analysis was performed to identify significantly altered metabolic pathways. The analysis was completed by comparing p metabolite data to the Small Molecule Pathway Database metabolite set based on normal human metabolic pathways. Enrichment ratio and significance of enrichment of pathways were calculated based on the number of metabolites detected within a specific pathway relative to the number of known metabolites in that pathway. The algorithm also considered relative intensity of metabolites in each group. Despite being named pathway ‘enrichment’ analysis, this method does not indicate upregulation or enrichment of pathways but determines pathways that are highly altered in the data.

Univariate receiver operating characteristic (ROC) analysis was performed to identify metabolic biomarkers that discriminate specific disease groups with high sensitivity and specificity. Mean levels of metabolites for each participant were used in the analyses. Strength of the discriminators was measured with area under the curve (AUC) values. Metabolites with AUC greater than 0.8 or 0.9 were considered as good or excellent discriminators, respectively. Uncharacterized metabolites were excluded from ROC analysis.

Multivariate ROC curve analysis was performed using random forest algorithm and automated feature selection for sample classification. This analysis identifies the most important features, which are used to build predictive models distinguishing disease groups. Performance of predictive models was evaluated using Monte Carlo cross-validation and measured by area under the curve (AUC) of the multivariate ROC and the confusion matrix calculated at a probability threshold of 0.5.

To determine associations between metabolite levels and tumor characteristics, participants with EC were stratified based on age (65 vs <65), histological grade (grade ½ EEC vs other EC), mismatch repair (MMR) protein status (MMR-deficient vs MMR-proficient), tumor size (>2 cm vs 2 cm), and myometrial invasion (present vs not present). Metabolite levels between those groups were compared using volcano plot analyses as described earlier. Spearman rank correlation analysis was performed to correlate metabolite levels to tumor size (measured in cm) and depth of myometrial invasion (measured in mm).

MetOrigin is freely available and was used to identify putative metabolic origins of identified metabolites. MetOrigin determines whether the metabolite is from host, microbiome, or potential co-metabolism.

Differences in demographic, socioeconomic, and other participant-related variables between disease groups were tested using the Kruskal-Wallis test for continuous variables and Fisher's exact test for categorical variables.

Study population: A total of 192 women undergoing hysterectomy were recruited and enrolled. Women were classified into four groups: benign conditions (n=108), endometrial hyperplasia (n=18), grade ½ EEC (n=53), and other EC subtypes (n=13) based on histopathological confirmation of biopsy samples. In some analyses EC is grouped as ‘EC All’ (n=63) and includes both grade ½ EEC and other EC. Clinical and demographic information for this cohort was previously described. Key demographic information is displayed in Table 3 and additional information relating to participant demographics and characteristics were also analyzed (data not shown). Overall, participants had a mean age of 51 years, mean body mass index (BMI) of 34.8 and were mostly Caucasian (75%). Participants diagnosed with EC were more often post-menopausal and on average were older with higher BMI (grade ½ EEC only) compared to participants with benign conditions.

Table 3 shows patient demographics for the cohort. Statistical analysis of participant demographics in the different groups analyzed. Values are n (%) unless stated as mean (SD). P-values were calculated using Kruskal-Wallis test for continuous variables and Fisher's exact test for categorical variables.

| | | | | Grade | | | | |
|---|---|---|---|---|---|---|---|---|
| | | | | 1/2 | Other | p-value | | |
| | All | Benign | Hyperplasia | EEC | EC | | Paired | |
| | (n = 192) | (n = 108) | (n = 18) | (n = 53) | (n = 13) | Overall | comparison | |
| Age (mean | 51.02 | 45.55 | 54.11 | 58.73 | 60.77 | <0.0001 | 1 vs 2 | 0.007 |
| (S.D)) (n = 192) | (12.45) | (10.01) | (13.35) | (11.82) | (8.06) | | 1 vs 3 | <0.0001 |
| | | | | | | | 1 vs 4 | <0.0001 |
| Race (n = 190) | | | | | | 0.16 | | |
| American | 15 | 5 | 1 | 8 | 1 | | | |
| Indian/Alaskan | (7.89) | (4.67) | (5.56) | (15.38) | (7.69) | | | |
| White/ | 142 | 78 | 16 | 37 | 11 | | | |
| Caucasian | (74.74) | (72.90) | (88.89) | (71.15) | (84.62) | | | |
| Black | 12 | 11 | 0 | 1 | 0 | | | |
| | (6.32) | (10.28) | (0.00) | (1.92) | (0.00) | | | |
| All Other | 21 | 13 | 1 | 6 | 1 | | | |
| | (11.05) | (12.15) | (5.56) | (11.54) | (7.69) | | | |
| Ethnicity (n = 191) | | | | | | 0.67 | | |
| Non-hispanic | 141 | 76 | 14 | 41 | 10 | | | |
| | (73.82) | (70.37) | (77.78) | (78.85) | (76.92) | | | |
| Hispanic | 50 | 32 | 4 | 11 | 3 | | | |
| | (26.13) | (29.63) | (22.22) | (21.15) | (23.08) | | | |
| Alcohol use (current) (n = 175) | | | | | | 0.11 | | |
| Yes | 72 | 50 | 7 | 11 | 4 | | | |
| | (41.41) | (49.50) | (43.75) | (24.44) | (30.77) | | | |
| No | 94 | 47 | 8 | 30 | 9 | | | |
| | (69.23) | (46.53) | (50.00) | (66.67) | (69.23) | | | |
| Quit | 9 | 4 | 1 | 4 | 0 | | | |
| | (5.14) | (3.96) | (6.25) | (8.89) | (0.00) | | | |
| Tobacco use (within last 6 months) (n = 184) | | | | | | 0.15 | | |
| Yes | 19 | 14 | 1 | 3 | 1 | | | |
| | (10.33) | (13.46) | (5.56) | (6.12) | (7.69) | | | |
| No | 55 | 34 | 4 | 13 | 4 | | | |
| | (29.89) | (32.69) | (22.22) | (26.53) | (30.77) | | | |
| Never | 89 | 47 | 12 | 22 | 8 | | | |
| | (48.37) | (45.19) | (66.67) | (44.90) | (61.54) | | | |
| Quit | 21 | 9 | 1 | 11 | 0 | | | |
| | (11.41) | (8.65) | (5.56) | (22.45) | (0.00) | | | |
| BMI (mean | 34.76 | 30.63 | 41.49 | 40.29 | 37.22 | <0.0001 | 1 vs 2 | <0.0001 |
| (SD)) (n = 192) | (10.16) | (7.54) | (7.45) | (11.07) | (12.76) | | 1 vs 3 | <0.0001 |
| | | | | | | | 1 vs 4 | 0.005 |
| BMI (n = 192) | | | | | | <0.0001 | 1 vs 2 | <0.0001 |
| <25 | 29 | 23 | 0 | 4 | 2 | | 1 vs 3 | <0.0001 |
| | (15.38) | (21.30) | (0.00) | (7.55) | (15.38) | | | |
| 25-29 | 47 | 38 | 1 | 6 | 2 | | | |
| | (24.38)) | (35.19) | (5.56) | (11.32) | (15.38) | | | |
| 30-34 | 30 | 19 | 2 | 6 | 3 | | | |
| | (15.63) | (17.59) | (11.11) | (11.32) | (23.08) | | | |
| ≥35 | 86 | 28 | 15 | 37 | 6 | | | |
| | (44.79) | (25.93) | (83.33) | (69.81) | (46.15) | | | |
| Menopausal status (n = 190) | | | | | | <0.0001 | 1 vs 2 | <0.0001 |
| Pre | 108 | 89 | 6 | 12 | 1 | | 1 vs 3 | <0.0001 |
| | (56.84) | (82.41) | (33.33) | (23.53) | (7.69) | | | |
| Post | 82 | 19 | 12 | 39 | 12 | | 1 vs 4 | <0.0001 |
| | (43.16) | (17.59) | (66.67) | (76.47) | (92.31) | | | |

Combined contraceptives (use in past 6 months)

| Hormonal (n = 138) | | | | | | 0.01 | 1 vs 2 | 0.02 |
|---|---|---|---|---|---|---|---|---|
| Yes | 36 | 26 | 0 | 5 | 5 | | 1 vs 3 | 0.06 |
| | (26.09) | (32.10) | (0.00) | (14.71) | (45.45) | | | |
| No | 102 | 55 | 12 | 29 | 6 | | 1 vs 4 | 0.01 |
| | (73.91) | (67.90) | (100.00) | (85.29) | (54.55) | | | |
| | | | | | | | 3 vs 4 | 0.048 |
| Non-hormonal (n = 64) | | | | | | 0.62 | | |
| Yes | 2 | 1 | 0 | 1 | 0 | | | |
| | (3.13) | (2.50) | (0.0) | (7.14) | (0.0) | | | |
| No | 62 | 39 | 6 | 13 | 4 | | | |
| | (96.88) | (97.50 | (100.0) | (92.86) | (100.0) | | | |
| Diabetes (n = 192) | | | | | | 0.006 | 1 vs 3 | 0.01 |
| Yes | 48 | 22 | 5 | 21 | 0 | | 2 vs 4 | 0.05 |
| | (25.00) | (20.37) | (27.78) | (39.62) | (0.0) | | | |
| No | 144 | 86 | 13 | 32 | 13 | | 3 vs 4 | 0.01 |
| | (75.00) | (79.63) | (72.22) | (60.38) | (100.0) | | | |
| Hypertension (n = 192) | | | | | | 0.002 | 1 vs 3 | 0.001 |
| Yes | 65 | 25 | 6 | 27 | 7 | | 1 vs 4 | 0.04 |
| | (33.85) | (23.15) | (33.33) | (50.94) | (53.85 | | | |
| No | 127 | 83 | 12 | 26 | 6 | | | |
| | (66.15) | (76.85) | (66.67) | (49.06) | (46.15) | | | |

*1 = Benign; 2 = Hyperplasia; 3 = Grade 1/2 EEC; 4 = Other EC

Global metabolomics reveals distinct cervicovaginal metabolic profiles for EC and benign participants: An untargeted global metabolomic approach and cervicovaginal sampling were used to assess the cervicovaginal metabolic profiles of women with EC, hyperplasia, and benign conditions. 920 metabolites were identified across the CVL samples, belonging to lipid (25%), amino acid (22%), xenobiotic (19%), nucleotide (7%), peptide (4%), carbohydrate (4%), cofactors and vitamins (4%), energy (1%), and partially characterized/uncharacterized (1% and 13%, respectively) superpathways. Metabolite origin investigation revealed that metabolites detected in CVL samples likely originated from a mix of host (2.6%), microbiota (9%), co-metabolism (23%), and other (64%; including drug, diet, environment and unknown sources).

Using fold change analysis and T-tests with FDR correction, significantly altered (q<0.05 and FC>2) metabolites were identified in EC groups compared to benign. Uncharacterized metabolites were not included in this analysis as they have limited utilization. The EC-All group had a total of 230 altered metabolites (167 downregulated and 63 upregulated) when compared to the benign group. The grade ½ EEC group had 204 altered metabolites (159 downregulated and 45 upregulated). The other EC group had 209 altered metabolites (101 downregulated and 108 upregulated). A large proportion of significantly upregulated metabolites in each EC group were lipids: EC-All had 41 (65.1% of all upregulated metabolites), grade ½ EEC had 25 (55.6%), and other EC groups had 86 (79.6%). Amino acids, peptides, and xenobiotics were significantly downregulated across EC. In EC-All, 60 downregulated amino acids (35.9% all downregulated metabolites), 25 downregulated peptides (15%), and 29 downregulated xenobiotics (1⁷0.4%). In grade ½ EEC there were 61 amino acids (38.4%), 23 peptides (14.5%), and 29 xenobiotics (18.2%) were identified. Finally, in other EC there were 40 amino acids (39.6%), 21 peptides (20.8%), and 5 xenobiotics (5%).

Hierarchical Clustering Analysis (HCA) stratified participants based on their metabolic profiles, producing a heatmap from the top 100 significant metabolites (ANOVA, q<0.05; data not shown). The analysis revealed two clusters were significantly (p<0.0001) different in disease distribution (diseases grouped as benign, hyperplasia, and EC all and clusters based on dendogram). Hyperplasia was distributed between both clusters, due to this hyperplasia did not take focus as a group in further analyses—hyperplasia participants were did not include and analysis continued for benign (n=108) vs grade ½ EEC (n=53) and other EC (n=13). Cluster 1 consisted of 64% EC, 29% benign, and 8% hyperplasia, and included all the other EC participants. Cluster 2 consisted of 76% benign, 13% EC, and 11% hyperplasia. All the EC participants within cluster 2 were grade ½ EEC. Cluster 1 showed upregulated lipids and cluster 2 showed upregulated amino acids, peptides, and xenobiotics. This analysis revealed global metabolic profiles can successfully distinguish participants with EC and benign conditions.

Metabolic profiling reveals that EC is associated with upregulation of lipids and downregulation of amino acids: Metabolomic investigation of CVL samples detected overall 228 lipids belonging to different classes: sterol lipids (14%), ketone bodies (1%), glycerolipids (4%), glycerophospholipids (22%), ceramides (4%), other sphingolipids (21%), long-chain fatty acids (4%), and other fatty acids (30%) (FIG. 1A). Lipids were significantly upregulated (q<0.05 and FC>2) in EC participants compared to benign participants, particularly glycerophospholipids (n=10 for EC all), other sphingolipids (n=5 for EC all), and other fatty acids (n=11 for EC all) (FIG. 1B).

Also, overall 206 amino acids were detected in CVL samples, belonging to a number of different pathways: leucine, isoleucine and valine metabolism (13%), histidine metabolism (11%), urea cycle (11%), methionine, cysteine, SAM and laurine metabolism (10%), lysine metabolism (10%), tyrosine metabolism (8%), tryptophan metabolism (7%), polyamine metabolism (6%), glutamate metabolism (5%), glycine, serine and threonine metabolism (4%), glutathione metabolism (4%), alanine and aspartate metabolism (4%), phenylalanine metabolism (3%), creatine metabolism (3%), guanidino and acetamido metabolism (1%) (FIG. 1C). Amino acids were significantly downregulated (q<0.05 and FC>2) in EC compared to benign conditions, particularly methionine, cysteine, SAM and laurine metabolism (n=11 for EC all), tyrosine metabolism (n=7 for EC all) and leucine, isoleucine and valine metabolism (n=6 for EC all) (FIG. 1D).

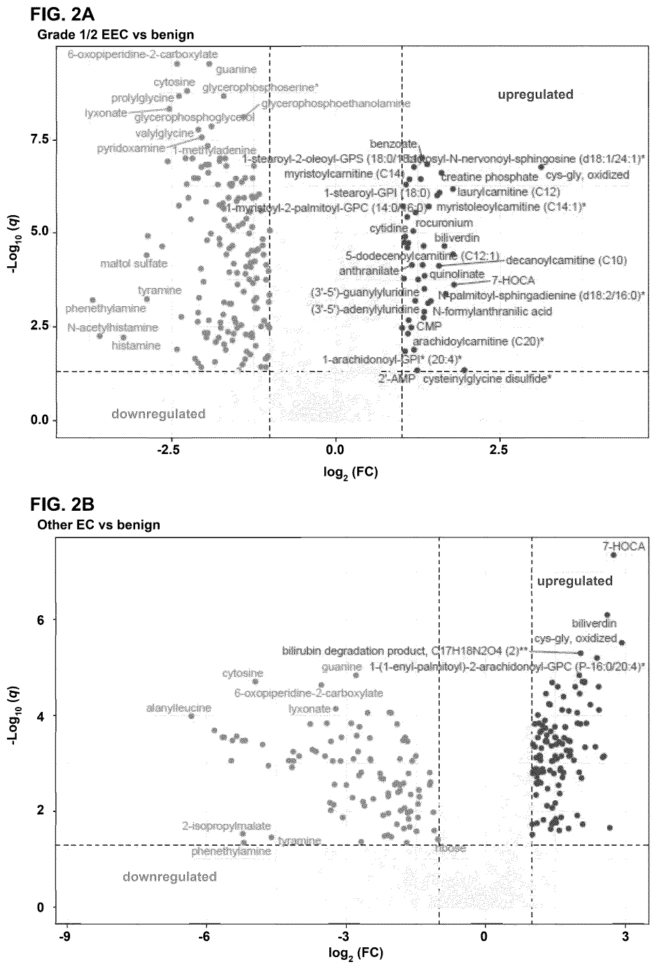

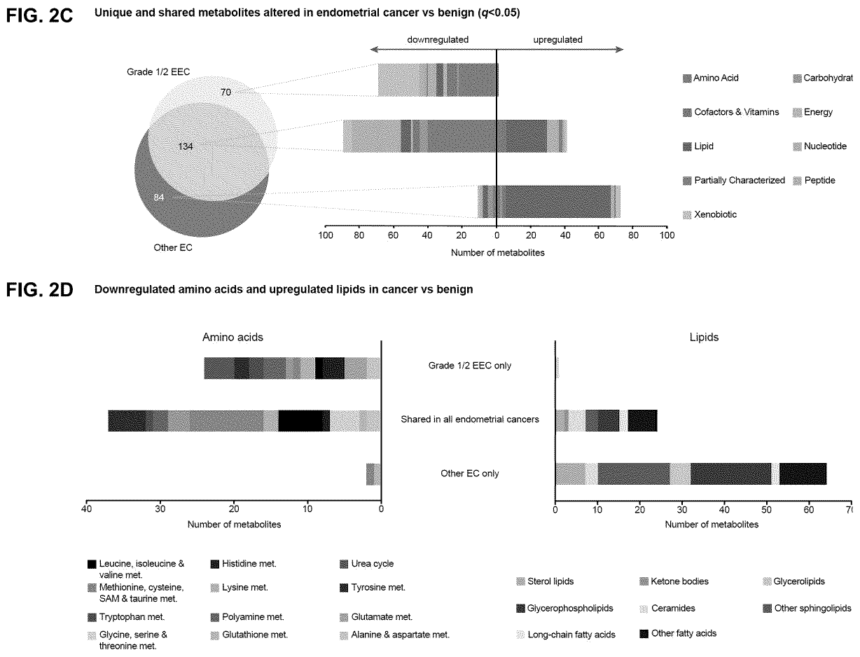

EC subtypes share many altered metabolites but have a number of uniquely altered metabolites: Significantly altered (q<0.05 and FC>2) metabolites were visualized on volcano plots to show up/downregulation. Comparison of grade ½ EEC to benign revealed 204 altered metabolites (45 upregulated and 159 downregulated). Comparison of other EC subtypes to benign revealed 209 altered metabolites (108 upregulated and 101 downregulated) (FIGS. 2A and 2B). Whilst both grade ½ EEC and other EC have a similar number of altered metabolites when compared to benign, other EC had more upregulated metabolites than grade ½ EEC, whereas grade ½ EEC had more downregulated metabolites than other EC. The Venn diagram shows overlap and distinct populations of significantly altered metabolites between grade ½ EEC and other EC. Grade ½ EEC has 70 uniquely altered metabolites (predominantly downregulated amino acids and xenobiotics), whereas other ECs have 84 uniquely altered metabolites (predominantly upregulated lipids). Both subgroups of EC share 134 altered metabolites, mostly upregulated lipids (n=24) and nucleotides (n=7) and downregulated amino acids (n=40), peptides (n=18), and nucleotides (n=11) (FIG. 2C). Other EC has a greater number of upregulated lipids than grade ½ compared to benign, particularly glycerophospholipids, other sphingolipids, and other fatty acids (FIG. 2D). Meanwhile, most downregulated amino acids are common among all subtypes of EC, with grade ½ EEC having a small number of uniquely altered amino acids when compared to benign (FIG. 2D).

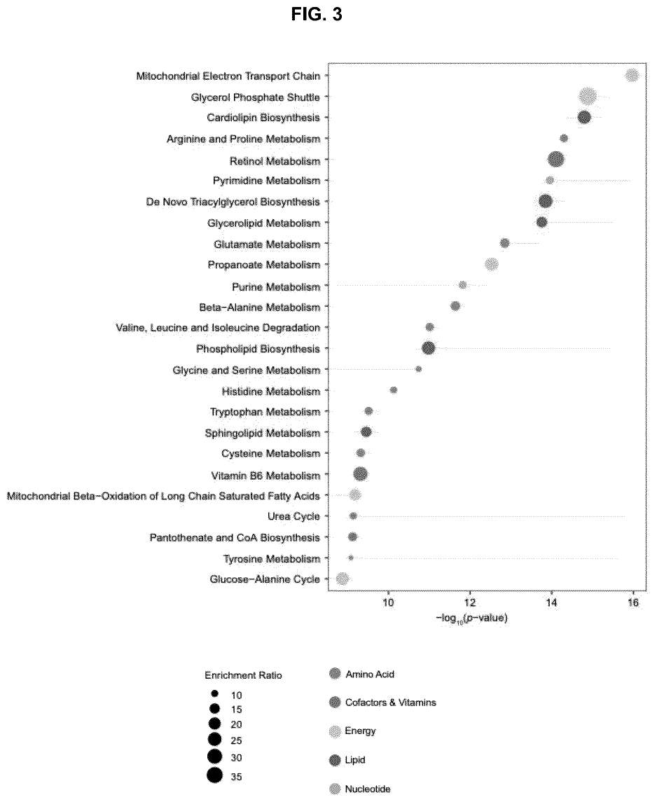

Altered metabolic pathways in EC: Pathway enrichment analysis was performed to identify which metabolic pathways are significantly altered in EC-All group compared to the benign group. 86 significantly (p<0.05) altered pathways were identified in EC-All vs benign; the top 25 pathways are shown in FIG. 3. Top pathways were associated with energy (n=5), lipid (n=5), amino acid (n=10), cofactors and vitamins (n=3), and nucleotide (n=2). Among the most significantly altered pathways were: energy pathways mitochondrial electron transport chain (METC) (p<0.0001) and glycerol phosphate shuttle (p<0.0001); lipid pathways cardiolipin biosynthesis (p<0.0001), de novo triacylglycerol biosynthesis (p<0.0001) and glycerolipid metabolism (p<0.0001); amino acid pathways arginine and proline metabolism (p<0.0001), glutamate metabolism (p<0.0001), histidine metabolism (p<0.0001), and tryptophan metabolism (p<0.0001); and nucleotide pathways pyrimidine metabolism (p<0.0001) and purine metabolism (p<0.0001).

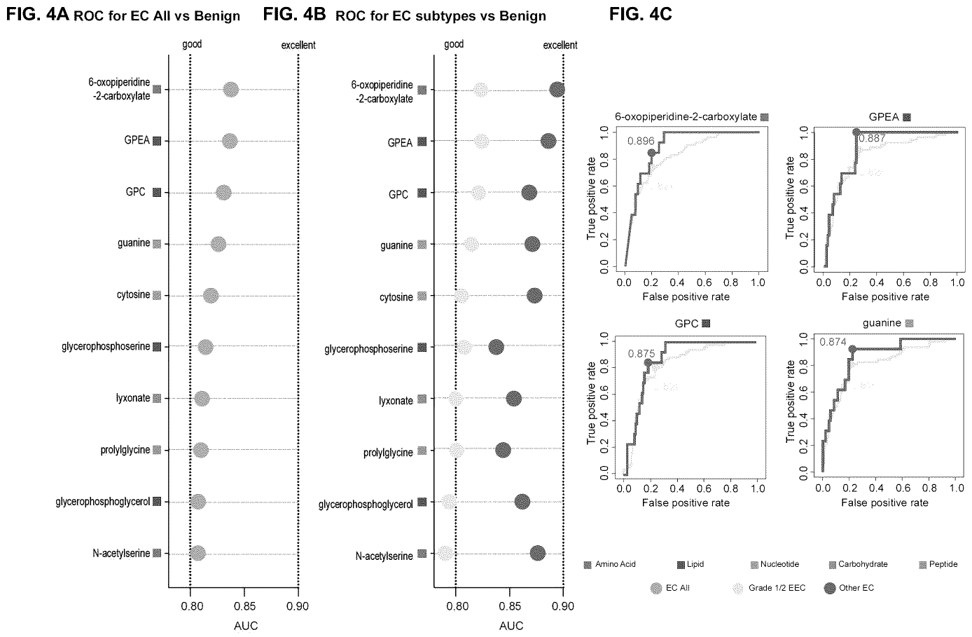

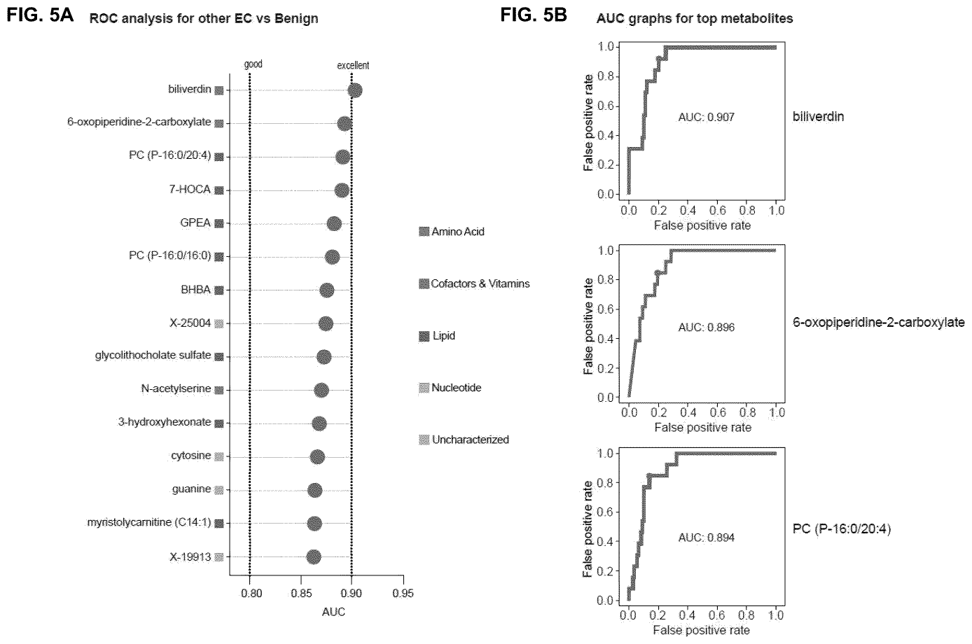

Metabolites in cervicovaginal lavages can discriminate EC participants from benign participants: An ROC analysis was performed to identify potential metabolic biomarkers that can distinguish participants with EC from participants with benign conditions with high specificity and sensitivity. When comparing the EC-All group to benign group, there were 10 metabolites that reached the a good biomarker threshold (AUC>0.8): 6-oxopiperidine-2-carboxylate (AUC=0.838), glycerophosphoethanolamine (GPEA) (AUC=0.37), glycerophosphocholine (GPC) (AUC=0.831), guanine (AUC=0.826), cytosine (AUC=0.819), glycerophosphoserine (AUC=0.814), lyxonate (AUC=0.810), prolylglycine (0.810), glycerophosphoglycerol (AUC=0.807), and N-acetylserine (AUC=0.807) (FIG. 4A). When comparing only grade ½ EEC to benign, there were only 6 metabolites that reached the AUC>0.8 threshold (all of which were among the 10 metabolites identified for EC-All) (FIG. 4B). ROC analysis comparing other EC to benign conditions revealed many potential biomarkers (147 metabolites for other EC subtypes, including biliverdin with an AUC>0.9, considered an excellent discriminator (FIGS. 5A and 5B)). Overall, metabolites exhibited higher sensitivity and specificity for other EC compared to grade ½ EEC (FIG. 4B). The top four biomarkers for EC-All vs benign are shown as AUC plots, with individual AUC values for grade ½ EEC and other EC depicted, which highlights these metabolites were more sensitive and specific for other EC compared to grade ½ EEC (FIG. 4C). Statistical analysis was performed with correction for BMI and age to ensure diagnostic markers are not signatures of age or obesity (Table 4). No loss in significance in any of the above diagnostic markers after correction.

Table 4 shows significance levels of metabolites after adjustment for age and BMI. A linear regression model was used, and p-values were adjusted using Dunnett adjustment. This is important because age and BMI do not impact the significance of these key metabolites; they are specific to endometrial cancer (not age or BMI); 1=Benign; 2=EMC All; 3=Low EMC; 4=Other EMC.

| | p-value Adjusted for Age and | | |
|---|---|---|---|
| | BMI | | |
| Metabolites | 2 vs 1 | 3 vs 1 | 4 vs 1 |
| 1-(1-enyl-palmitoyl)-2-palmitoyl-GPC (P-16:0/16:0)* | <0.0001 | 0.0003 | <0.0001 |
| 1-palmitoyl-2-dihomo-linolenoyl-GPC(16:0/20:3n3 or 6)* | <0.0001 | 0.0004 | 0.0009 |
| 1-stearoyl-2-arachidonoyl-GPC (18:0/20:4) | 0.0002 | 0.0039 | 0.001 |
| 1-stearoyl-2-arachidonoyl-GPI (18:0/20:4) | <0.0001 | <0.0001 | <0.0001 |
| 2′-deoxyuridine | 0.9988 | 0.974 | 0.8403 |
| 3-hydroxybutyrate (BHBA) | <0.0001 | 0.0002 | <0.0001 |
| 3-hydroxyhexanoate | <0.0001 | <0.0001 | <0.0001 |
| 3-methyl-2-oxobutyrate | 0.3932 | 0.0011 | 0.012 |
| 5-methyluridine (ribothymidine) | 0.0002 | 0.6642 | 0.7959 |
| 6-oxopiperidine-2-carboxylate | <0.0001 | <0.0001 | 0.0004 |
| 7-alpha-hydroxy-3-oxo-4-cholestenoate (7-Hoca) | <0.0001 | 0.0072 | <0.0001 |
| adenosine | <0.0001 | 0.0012 | 0.0015 |
| adenosine 5′-monophosphate (AMP) | 0.2179 | 0.3993 | 0.7241 |
| biliverdin | <0.0001 | 0.0001 | <0.0001 |
| carnosine | 0.0231 | 0.0512 | 0.3723 |
| cysteine s-sulfate | 0.0019 | 0.0134 | 0.0196 |
| cytidine diphosphate | 0.1413 | 0.3715 | 0.3359 |
| cytosine | <0.0001 | <0.0001 | 0.0004 |
| dihomo-linoleate (20:2n6) | <0.0001 | 0.0011 | 0.0016 |
| dihomo-linolenate (20:3n3 or n6) | 0.0014 | 0.0072 | 0.0395 |
| fructose 1,6-diphosphate/glucose 1,6-diphosphate/myo- | <.0001 | 0.0003 | 0.0073 |
| inositol diphosphates | | | |
| gabapentin | 0.0002 | 0.0021 | 0.0025 |
| glycerophosphoethanolamine | <0.0001 | <0.0001 | 0.0005 |
| glycerophosphoglycerol | <0.0001 | <0.0001 | 0.0011 |
| glycerophosphorylcholine (GPC) | <.0001 | <0.0001 | 0.0029 |
| glycerophosphoserine* | <0.0001 | <0.0001 | 0.0008 |
| glycolithocholate sulfate* | 0.0043 | 0.0579 | 0.0024 |
| guanine | <0.0001 | <0.0001 | <0.0001 |
| heptadecasphingosine (d17:1) | <0.0001 | <0.0001 | 0.0074 |
| lyxonate | <0.0001 | <0.0001 | <0.0001 |
| myristoleoylcarnitine (C14:1)* | <0.0001 | 0.0006 | <0.0001 |
| myristoylcarnitine (C14) | <0.0001 | <0.0001 | 0.0005 |
| N-acetylglucosamine/N-acetylgalactosamine | 0.1611 | 0.4055 | 0.3707 |
| N-acetylserine | 0.0002 | 0.0016 | 0.0077 |
| N-acetyltaurine | 0.3119 | 0.4819 | 0.9132 |
| N-palmitoyl-sphinganine (d18:0/16:0) | 0.0247 | 0.0741 | 0.2179 |
| palmitoleoylcarnitine (C16:1)* | <0.0001 | 0.0007 | 0.0003 |
| prolylglycine | <0.0001 | <0.0001 | 0.0001 |
| pyridoxamine | <0.0001 | <0.0001 | 0.0004 |
| S-adenosylmethionine (SAM) | <.0001 | <0.0001 | 0.0056 |
| spermine | <0.0001 | 0.0004 | 0.004 |
| sphingomyelin (d18:1/18:1, d18:2/18:0) | 0.0001 | 0.0035 | 0.0001 |
| sphingomyelin (d18:1/20:1, d18:2/20:0)* | 0.0002 | 0.0053 | 0.0001 |
| sphingomyelin (d18:1/21:0, d17:1/22:0, d16:1/23:0)* | 0.0005 | 0.0166 | 0.0001 |
| stearoyl sphingomyelin (d18:1/18:0) | 0.0006 | 0.0105 | 0.0012 |
| uridine | 0.106 | 0.2396 | 0.473 |

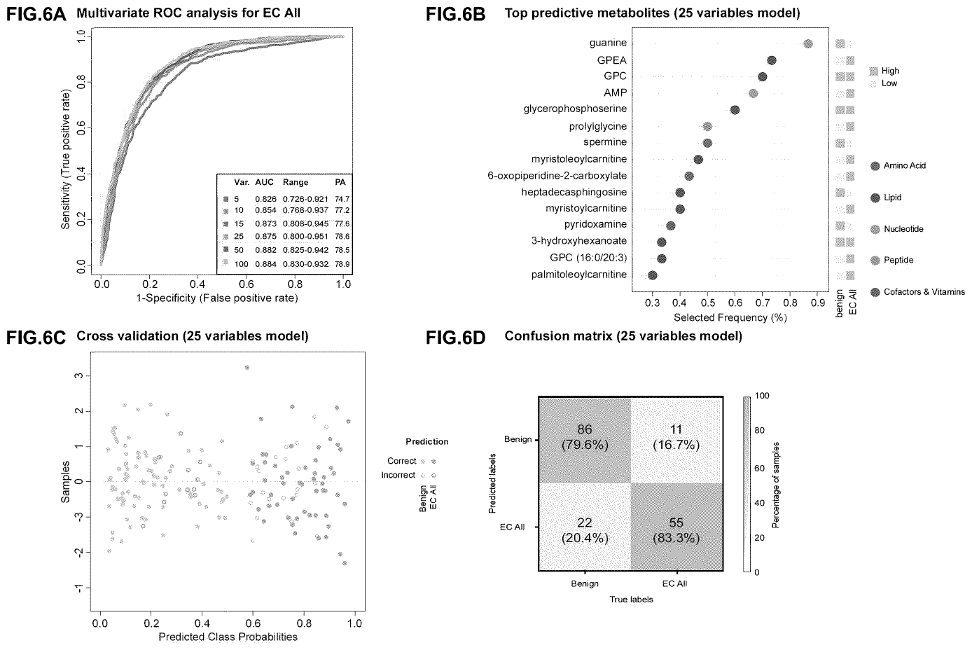

Machine learning-based multivariate models accurately predict EC from benign conditions: A multivariate ROC approach based on a random forest algorithm was used to predict disease groups. This may be useful as an individual biomarker may be elevated in other conditions; therefore, a combination of multiple metabolites may exhibit higher sensitivity and specificity for EC detection. A multivariate ROC analysis was conducted for EC-All vs benign using a range of features from 5 to 100 metabolites, chosen by the machine learning algorithm to build multivariate models (FIG. 6A). The average AUC for all the numbers of features was >0.8, and therefore these models were considered good discriminators, with five-feature and 100-feature models giving average AUCs of 0.826 and 0.884, respectively. A multivariate model with 25 metabolic features presents potential for a good diagnostic tool, with an AUC range of 0.800-0.951; predictive accuracy of each method remained consistent with 5 features having a predictive accuracy of 74.7% and 100 features with 78.9% (FIG. 6A). A 25-feature model based on the predictive accuracies was used, as 25 is a manageable number of metabolites to be measured for a diagnostic test. The top 15 predictive features that were most frequently used to create the model by random forest included mostly lipids (GPEA, GPC, glycerophosphoserine, myristoleoylcarnitine, heptadecasphingosine, myristoylcarntine, 3-hydroxyhexonate, GPC (16:0/20:3), and palmitoleoylcarntine), and some nucleotides (guanine, AMP), amino acids (spermine and 6-oxopiperidine-2-carboxylate), peptides (prolylglycine), and cofactors and vitamins (pyridoxamine) (FIG. 6B). Many metabolites used to build this multivariate model were also identified in the univariate ROC analyses, including guanine, GPEA, GPC, and prolylglycine. This model of 25-variables shows high predictive accuracy of 78.6% based on cross-validation, with many participants correctly classified into their disease group (FIG. 6C). A confusion matrix shows the times each sample obtained was classified correctly. For EC-All, 83.3% (n=55) of participants were correctly classified as EC-All, and only 16.7% (n=11) were incorrectly classified as benign. For benign participants, 79.6% (n=86) were correctly classified as benign, and 20.4% (n=22) were incorrectly classified as EC-All (FIG. 6D).

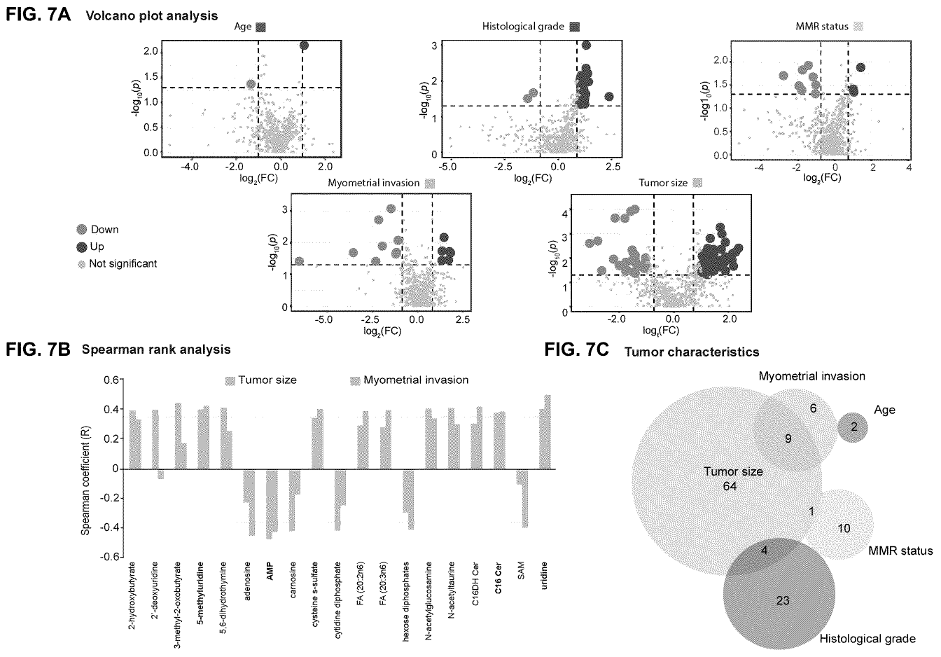

Cervicovaginal metabolite levels are reflective of tumor characteristics: EC participants were grouped based on tumor characteristics: histological grade (other EC vs grade ½ EEC), MMR status (MMR-deficient vs MMR-proficient), tumor size (>2 cm vs 2 cm), and myometrial invasion (present vs not present). In addition, participants with EC were stratified based on age (65 years vs <65 years), as increased age is a risk factor of EC. Volcano plots show significantly upregulated/downregulated (p<0.05 and FC>2) metabolites associated with each tumor characteristic (FIG. 7A). For age, one downregulated metabolite (nicotinate ribonucleoside) and one upregulated metabolite (N6-methyllysine) were identified. For histological grade, 25 upregulated metabolites and two downregulated metabolites (2-methylbutyrylcarnitine (C5), and homocysteine) were identified. For MMR status, three were upregulated (decanoylcarnitine (C10), laurylcarnitine (C12), and octonolycarnitine (C8)) and eight were downregulated. For myometrial invasion, nine were downregulated and six were upregulated, including many lipids. Finally, for tumor size, 25 downregulated and 53 upregulated, 56% of which were lipids (n=44) were identified. In addition, Spearman rank correlation analysis was used to show the strength of the relationship between individual metabolite levels and tumor size (measured in cm) and depth of myometrial invasion (measured in mm). The top 10 metabolites identified that were significantly correlated with tumor size and/or myometrial invasion (with a mix of some positively and some negatively correlating) included amino acids, carbohydrates, lipids, and nucleotides. 5-methyluridine, AMP, C16 Ceramide (Cer), and uridine all correlated with both tumor size and myometrial invasion (mix of negative and positive correlation) (FIG. 7B). All results from Spearman rank analysis are in Table 5. The Venn diagram illustrates the number of metabolites that were unique or shared between tumor characteristics (FIG. 7C). Age did not have any shared altered metabolites with any tumor characteristics investigated. Myometrial invasion and tumor size shared nine metabolites (AMP, 2,3-diphosphoglycerate, C16DH Cer, argininate, C16 Cer, (3′-5′)-cytidyluridine, C14 Cer, N-stearoyl-sphingosine (d18:1/18:0), PC-(P-16:0/16:0)), MMR status and tumor size shared adenosine only, and histological grade and tumor size shared four metabolites (homocysteine, 1-(1-enyl-palmitoyl)-2-linoleoyl-GPC (P-16:0/18:2), BHBA, and PC-(P-16:0/20:4)).

Table 5 shows specific metabolites listed that were used to create the volcano plot information for FIG. 7A and FIG. 7C. R=Spearman correlation.

| | Myometrial | | | |
|---|---|---|---|---|
| | invasion | | Tumor size | |
| Metabolite name | R | p-value | R | p-value |
| 1-(1-enyl-palmitoyl)-2-linoleoyl-GPC (P-16:0/18:2) | | | 0.3008 | 0.0175 |
| 1-(1-enyl-palmitoyl)-2-linoleoyl-GPE (P-16:0/18:2) | 0.3478 | 0.0065 | 0.3141 | 0.0129 |
| 1-(1-enyl-palmitoyl)-2-oleoyl-GPC (P-16:0/18:1) | | | 0.3213 | 0.0109 |
| 1-(1-enyl-palmitoyl)-2-oleoyl-GPE (P-16:0/18:1) | 0.3357 | 0.0087 | 0.3302 | 0.0088 |
| 1-(1-enyl-palmitoyl)-2-palmitoyl-GPC (P-16:0/16:0) | 0.3587 | 0.0049 | 0.3703 | 0.003 |
| 1-(1-enyl-stearoyl)-2-arachidonoyl-GPE (P-18:0/20:4) | | | 0.371 | 0.003 |
| 1-(1-enyl-stearoyl)-2-oleoyl-GPE (P-18:0/18:1) | | | 0.3611 | 0.0039 |
| 1-(1-enyl-stearoyl)-GPE (P-18:0) | 0.321 | 0.0124 | 0.3103 | 0.0141 |
| 1-arachidonoyl-GPE (20:4n6) | 0.3095 | 0.0161 | | |
| 1-arachidonoyl-GPI* (20:4) | 0.3386 | 0.0081 | | |
| 1-dihomo-linolenylglycerol (20:3) | 0.3558 | 0.0053 | 0.3486 | 0.0055 |
| 1-linoleoyl-GPE (18:2) | 0.3338 | 0.0092 | | |
| 1-oleoyl-GPE (18:1) | 0.3746 | 0.0032 | | |
| 1-oleoylglycerol (18:1) | 0.3202 | 0.0126 | | |
| 1-palmitoyl-2-arachidonoyl-GPE (16:0/20:4) | | | 0.3143 | 0.0129 |
| 1-palmitoyl-2-linoleoyl-GPE (16:0/18:2) | | | 0.3086 | 0.0147 |
| 1-palmitoyl-2-oleoyl-GPE (16:0/18:1) | | | 0.3093 | 0.0144 |
| 1-palmitoyl-2-oleoyl-GPS (16:0/18:1) | 0.3364 | 0.0086 | | |
| 1-palmitoyl-GPE (16:0) | 0.3139 | 0.0146 | | |
| 1-palmitoylglycerol (16:0) | 0.3661 | 0.004 | 0.3013 | 0.0173 |
| 1-stearoyl-2-oleoyl-GPE (18:0/18:1) | 0.3095 | 0.0161 | 0.3639 | 0.0036 |
| 1-stearoyl-2-oleoyl-GPS (18:0/18:1) | 0.3406 | 0.0077 | 0.3622 | 0.0038 |
| 1-stearoyl-GPI (18:0) | 0.3417 | 0.0075 | | |
| 1-stearoyl-GPS (18:0) | 0.3352 | 0.0088 | | |
| 12-dipalmitoyl-GPC (16:0/16:0) | | | 0.3066 | 0.0154 |
| 2-aminoadipate | −0.3615 | 0.0045 | | |
| 2-hydroxybutyrate/2-hydroxyisobutyrate | 0.3302 | 0.01 | 0.3916 | 0.0016 |
| 2-linoleoylglycerol (18:2) | | | 0.3135 | 0.0131 |
| 2,3-diphosphoglycerate | −0.3747 | 0.0032 | | |
| 2′-AMP | −0.3175 | 0.0134 | | |
| 2′-deoxyuridine | | | 0.3983 | 0.0013 |
| 2′-O-methylguanosine | | | 0.3574 | 0.0043 |
| 3-hydroxyhexanoate | 0.3121 | 0.0152 | 0.3504 | 0.0052 |
| 3-hydroxyoctanoate | | | 0.3459 | 0.0059 |
| 3-methyl-2-oxobutyrate | | | 0.4412 | 0.0003 |
| 3-methyl-2-oxovalerate | | | 0.3816 | 0.0022 |
| 4-hydroxyglutamate | | | −0.3351 | 0.0078 |
| 4-methyl-2-oxopentanoate | | | 0.3539 | 0.0048 |
| 5-hydroxylysine | 0.3626 | 0.0044 | | |
| 5-methyluridine (ribothymidine) | 0.4218 | 0.0008 | 0.3986 | 0.0013 |
| 56-dihydrothymine | | | 0.4111 | 0.0009 |
| acetylphosphate | −0.3026 | 0.0188 | | |
| adenine | −0.3353 | 0.0088 | | |
| adenosine | −0.4427 | 0.0004 | | |
| alpha-hydroxyisocaproate | −0.336 | 0.0087 | | |
| AMP | −0.4164 | 0.0009 | −0.4644 | 0.0001 |
| anthranilate | | | 0.3006 | 0.0176 |
| arachidate (20:0) | 0.3176 | 0.0134 | | |
| bilirubin degradation product C17H18N2O4 (2) | | | 0.331 | 0.0086 |
| carnosine | | | −0.4104 | 0.0009 |
| CDP-choline | | | −0.3636 | 0.0037 |
| CDP-ethanolamine | | | −0.3696 | 0.0031 |
| ceramide (d18:1/14:0 d16:1/16:0) | | | 0.3078 | 0.0149 |
| ceramide (d18:1/14:0, d16:1/16:0) | 0.3622 | 0.0045 | | |
| ceramide (d18:1/17:0, d17:1/18:0) | 0.352 | 0.0058 | | |
| cholesterol | | | 0.3111 | 0.0139 |
| choline | | | 0.3528 | 0.0049 |
| citrate | 0.3617 | 0.0045 | | |
| cys-gly oxidized | | | 0.3385 | 0.0071 |
| cysteine s-sulfate | 0.4015 | 0.0015 | 0.3404 | 0.0068 |
| cysteinylglycine | −0.3357 | 0.0087 | | |
| cysteinylglycine disulfide | 0.3077 | 0.0168 | 0.3398 | 0.0069 |
| cytidine diphosphate | | | −0.408 | 0.001 |
| decanoylcarnitine (C10) | 0.3 | 0.0199 | | |
| dihomolinoleate (20:2n6) | 0.3869 | 0.0023 | | |
| dihomolinolenate (20:3n3 or 3n6) | 0.3931 | 0.0019 | | |
| erucate (22:1n9) | 0.3008 | 0.0195 | | |
| Fibrinopeptide A | 0.323 | 0.0118 | | |
| Fibrinopeptide A, phosphono-ser(3) | 0.3227 | 0.0119 | | |
| glycerol | 0.3158 | 0.014 | | |
| glycosyl-N-palmitoyl-sphingosine (d18:1/16:0) | 0.3028 | 0.0187 | 0.379 | 0.0024 |
| guanosine | | | 0.3174 | 0.0119 |
| hexanoylcarnitine (C6) | 0.331 | 0.0098 | | |
| homogentisate | −0.3057 | 0.0176 | | |
| inosine | 0.3102 | 0.0159 | | |
| Isobar: hexose diphosphates | −0.4031 | 0.0014 | | |
| isobutyrylglycine (C4) | 0.3437 | 0.0072 | | |
| lactosyl-N-nervonoyl-sphingosine (d18:1/24:1) | | | 0.3221 | 0.0107 |
| lactosyl-N-palmitoyl-sphingosine (d18:1/16:0) | | | 0.3215 | 0.0108 |
| lignoceroyl sphingomyelin (d18:1/24:0) | | | 0.3051 | 0.0159 |
| malonylcarnitine | 0.3353 | 0.0088 | | |
| mevalonate | −0.3281 | 0.0105 | | |
| mevalonolactone | −0.3577 | 0.005 | | |
| myristoyl dihydrosphingomyelin (d18:0/14:0) | 0.3451 | 0.0069 | | |
| myristoylcarnitine (C14) | 0.3377 | 0.0083 | 0.3015 | 0.0173 |
| N-acetyl-cadaverine | −0.3123 | 0.0151 | | |
| N-acetylaspartate (NAA) | 0.3139 | 0.0146 | | |
| N-acetylglucosamine/N-acetylgalactosamine | 0.3359 | 0.0087 | 0.4023 | 0.0012 |
| N-acetylglycine | 0.3189 | 0.013 | 0.3331 | 0.0082 |
| N-acetyltaurine | | | 0.4065 | 0.001 |
| N-alpha-acetylornithine | 0.3205 | 0.0125 | | |
| N-palmitoyl-sphinganine (d18:0/16:0) | 0.4175 | 0.0009 | 0.3023 | 0.0169 |
| N-palmitoyl-sphingosine (d18:1/16:0) | 0.384 | 0.0025 | 0.3733 | 0.0028 |
| N-stearoyl-sphingosine (d18:1/18:0) | 0.3283 | 0.0104 | 0.329 | 0.009 |
| N6-methyllysine | 0.308 | 0.0167 | | |
| NAD+ | | | −0.3361 | 0.0076 |
| nicotinamide ribonucleotide (NMN) | | | 0.3097 | 0.0143 |
| oleoylcarnitine (C18:1) | | | 0.3063 | 0.0155 |
| oxalate (ethanedioate) | 0.3157 | 0.014 | 0.3176 | 0.0119 |
| p-cresol glucuronide | | | −0.3661 | 0.0034 |
| palmitoyl dihydrosphingomyelin (d18:0/16:0) | 0.3277 | 0.0106 | 0.3042 | 0.0162 |
| palmitoylcarnitine (C16) | | | 0.3139 | 0.013 |
| phenylacetylglutamine | | | −0.3161 | 0.0123 |
| prostaglandin A2 | 0.3034 | 0.0185 | | |
| S-adenosylmethionine (SAM) | −0.3877 | 0.0022 | | |
| sphingomyelin (d17:1/16:0 d18:1/15:0 d16:1/17:0) | | | 0.3184 | 0.0117 |
| sphingomyelin (d18:0/20:0, d16:0/22:0) | 0.3016 | 0.0192 | | |
| sphingomyelin (d18:1/24:1 d18:2/24:0) | | | 0.305 | 0.0159 |
| stearate (18:0) | 0.3142 | 0.0145 | | |
| UDP-glucose | −0.3335 | 0.0092 | −0.3415 | 0.0066 |
| UDP-glucuronate | | | −0.3168 | 0.0121 |
| UDP-N-acetylglucosamine/galactosamine | | | −0.3742 | 0.0027 |
| uridine | 0.495 | <0.0001 | 0.3988 | 0.0013 |
| uridine 2′-monophosphate (2′-UMP) | −0.3009 | 0.0195 | | |
| valylglutamine | −0.3255 | 0.0111 | | |
| xanthine | | | 0.3581 | 0.0043 |
| 1-(1-enyl-palmitoyl)-2-arachidonoyl-GPE (P-16:0/20:4) | | | 0.3351 | 0.0078 |

### Example 2

The following is a non-limiting example of the present invention. It is to be understood that said example is not intended to limit the present invention in any way. Equivalents or substitutes are within the scope of the present invention.

Study population and specimen collection: Briefly, 192 women undergoing hysterectomy for benign or malignant conditions were enrolled at three clinical sites in the Phoenix, AZ metropolitan area. The patients were stratified into three disease groups: endometrial carcinoma (n=66), endometrial hyperplasia (n=18) and benign controls (n=108) based on histopathological examinations of uterine tissue post hysterectomy. Clinical specimens, including vaginal swabs and CVL samples, were collected by a physician in an operating room prior to hysterectomy. Samples were frozen within 1 hour of collection and stored at −80° C. prior to downstream analyses.

Ultra-high performance liquid chromatography-tandem mass spectrometry: Metabolites in vaginal swab samples were quantified using a global metabolomics platform at Metabolon Inc. as described previously. The platform utilizes ultra-performance liquid chromatography-tandem mass spectrometry (UPLC-MS/MS), several recovery standards for quality control purposes, and multiple extraction methods optimized for recovering chemically diverse metabolites. Prior to analysis, samples were treated with methanol under vigorous shaking for 2 min, followed by centrifugation to remove protein content. The resulting extracts were divided into multiple fractions, dried using TurboVap (Zymark), and stored overnight under nitrogen. The dried sample extracts were reconstituted in appropriate solvents and analyzed by four UPLC-MS/MS methods: two separate reverse phase (RP)/UPLC-MS/MS methods with positive ion mode electrospray ionization (ESI), a RP/UPLC-MS/MS method with negative ion mode ESI, and a HILIC/UPLC-MS/MS method with negative ion mode ESI. All methods utilized a Waters ACQUITY UPLC and a Thermo Scientific Q-exactive high-resolution/accurate-mass spectrometer, interfaced with a heated ESI (HESI-II) source and an Orbitrap mass analyzer, operated at 35,000 mass resolution. The samples were analyzed using (1) acidic positive ion conditions, chromatographically optimized for more hydrophilic compounds following a gradient elution from a C18 column (Waters UPLC BEH C18-2.1×100 mm, 1.7 μm) with water, methanol, 0.05% perfluoropentanoic acid, and 0.1% formic acid; (2) acidic positive ion conditions, chromatographically optimized for more hydrophobic compounds, following a gradient elution from the same aforementioned C18 column with methanol, acetonitrile, water, 0.05% perfluoropentanoic acid, and 0.01% formic acid; (3) basic negative ion optimized conditions following a gradient elution from a separate C18 column with methanol, water, and 6.5 mM ammonium bicarbonate at pH 8; (4) negative ionization following a gradient elution from a HILIC column (Waters UPLC BEH Amide 2.1×150 mm, 1.7 μm) with water, acetonitrile, and 10 mM ammonium formate at pH 10.8. The MS analysis alternated between MS and data-dependent MSn scans using dynamic exclusion. The scan range varied slightly between methods but covered 70-1000 m/z. A Waters ACQUITY UPLC and a Thermo Scientific Q-exactive high resolution/accurate mass spectrometer, interfaced with a heated electrospray ionization (HESI-II) source and Orbitrap mass analyzer operated at 35,000 mass resolution, were used in the analyses. Raw data files were archived and extracted as described below.

Metabolomics quality assurance: Several types of controls were analyzed together with the experimental samples: a pooled matrix sample generated by taking a small volume of each experimental sample served as a technical replicate throughout the data set; extracted water samples served as process blanks; and a cocktail of quality control (QC) standards that were carefully chosen not to interfere with the measurement of endogenous compounds were spiked into every analyzed sample. This allowed instrument performance monitoring and aided chromatographic alignment. Instrument variability was determined by calculating the median relative standard deviation (RSD) of the internal standards added to each sample prior to injection into the mass spectrometers. Overall process variability was determined by calculating the median RSD for all endogenous metabolites present in 100% of the pooled matrix samples. Experimental samples were randomized across the platform run with QC samples spaced evenly among the injections. Median RSD values for instrument and process variability were 5% and 8%, respectively, and met Metabolon's acceptance criteria.

Metabolite identification and quantification: Raw data were extracted, peak-identified, and QC processed using a combination of Metabolon-developed software services. Compounds were identified by comparison to library entries of 5,400 purified standards or recurrent unknown entities. Metabolon maintains a library based on authenticated standards that contains the retention time/index (RI), mass to charge ratio (m/z), and fragmentation data on all molecules present in the library. Biochemical identifications were based on three criteria: retention index within a narrow RI window of the proposed identification, accurate mass match to the library ±10 ppm, and the MS/MS forward and reverse scores between the experimental data and authentic standards. The MS/MS scores were based on a comparison of the ions present in the experimental spectrum to the ions present in the library spectrum. Peaks of identified compounds were quantified using the area under the curve (AUC). To remove batch variability, for each metabolite, the values in the experimental samples were divided by the median of those samples in each instrument batch, giving each batch and thus the metabolite a median of one. Missing values were imputed with the minimum value across all batches for each metabolite in the median scaled data.

Statistical analyses: Statistical differences between the disease groups were assessed using Kruskal-Wallis test for continuous variables and Fisher's exact test or chi-square test for categorical variables. The metabolomic data were analyzed using MetaboAnalyst 6.0. Log₁₀-transformation and autoscaling were applied to normalize the data. Partial least squares discriminant analysis (PLS-DA) was performed to reduce data dimensionality and visualize clusters of samples by disease group. The significance of cluster separation was assessed using permutation testing. The unsupervised hierarchical clustering analysis (HCA) was performed to show the relationship between metabolic profiles and disease status. The clustering was based on Euclidean distance and the Ward linkage algorithm. The data were visualized using a heatmap and dendrograms displaying the metabolic composition of samples and the similarity or dissimilarity between the samples based on their metabolic profiles. The difference in sample distribution between identified clusters was evaluated using Fisher's exact test. Differential abundance of metabolites was assessed using a combination of the fold change (FC) and t-test analyses. FC was calculated as the ratio between two group means, and the significance was measured by an unpaired t-test with false discovery rate (FDR) correction. Metabolites with FC>2 or <−2 and q-value <0.05 were considered as significantly enriched or depleted between the tested groups. Volcano plots were utilized to represent both the magnitude of change (FC) and the statistical significance (p-value or q-value) of all metabolites in the tested groups. Metabolite set enrichment analysis (MSEA) was performed to identify alterations of functionally related metabolite sets between the disease groups. The library of normal human metabolic pathways based on the Small Molecule Pathway Database (SMPDB) and the metabolite relative intensity table was used for the quantitative enrichment analysis (QEA). Enrichment ratios for each metabolic pathway were computed by dividing the number of hits (significant metabolites) within a particular metabolic pathway by the expected number of hits based on random chance. Q-statistics for each metabolite set were estimated using a generalized linear model. The receiver operating characteristic (ROC) analysis was used to identify potential metabolic biomarkers discriminating the disease groups at high sensitivity and specificity. The strength of discriminatory potential was assessed using AUC values. Metabolites with AUC greater than or equal to 0.8 were considered as good discriminators. Multivariable logistic regression models were evaluated for the accurate prediction of the disease groups using different combinations of metabolites. The models were built using metabolic features selected based on the least absolute shrinkage and selection operator (LASSO) modeling. The performance of the predictive models was evaluated using Monte Carlo cross-validation (using ⅔ of samples for model training and ⅓ of samples for model testing), and measured by AUC values from multivariable ROC analyses and confusion matrices calculated at a probability threshold of 0.5. Predictive accuracy, sensitivity, specificity, Youden's index (J) and Cohen's kappa were reported for each tested model.

Study population: Briefly, 192 patients undergoing hysterectomy for malignant or benign conditions were stratified into three disease groups: EC (n=66), endometrial hyperplasia (n=18), and benign controls (n=108). The disease classification was based on histopathological examination of uterine tissue post-surgery. Patients' average age was 51 years old (ranging from 29 to 85 years) and average body mass index (BMI) was 34.8 kg/m² (ranging from 19.3 to 67.4 kg/m²). The majority of patients were White/Caucasian (74.7%), followed by American Indian/Alaska Native (7.9%) and Black/African American (6.3%). Regarding ethnicity, 26.2% of participants identified themselves as Hispanic/Latina. There were significant differences between the groups regarding age, menopause status, and BMI, with EC patients being older, mostly postmenopausal, and having higher BMI compared to benign patients (Table 6).

Table 6 shows patient demographics. Statistical differences between the groups were assessed using Kruskal-Wallis test for continuous variables and Fisher's exact test or chi square test for categorical variables.

| | | | | | Endometrial | | Endometrial | | |
|---|---|---|---|---|---|---|---|---|---|
| | All | | Benign | | Hyperplasia | | Cancer | | |
| | (n = 192) | | (n = 108) | | (n = 18) | | (n = 66) | | p-value |
| Age [mean (SD)] | 51.01 | (12.45) | 45.56 | (10.02) | 54.11 | (13.36) | 59.14 | (11.16) | <0.0001 |
| Menopause Status* | | | | | | | | | |
| [n (%)] | | | | | | | | | |
| premenopause | 108 | (56.84) | 89 | (82.41) | 6 | (33.33) | 13 | (20.31) | <0.0001 |
| postmenopause | 82 | (43.16) | 19 | (17.59) | 12 | (66.67) | 51 | (79.7) | |
| Race | | | | | | | | | |
| White/Caucasian | 142 | (74.74) | 78 | (72.90) | 16 | (88.89) | 48 | (73.85) | 0.087 |
| American Indian/ | 15 | (7.89) | 5 | (4.67) | 1 | (5.56) | 9 | (13.85) | |
| Alaska Native | | | | | | | | | |
| Black/African | 12 | (6.32) | 11 | (10.28) | 0 | (0.00) | 1 | (1.54) | |
| American | | | | | | | | | |
| Other | 21 | (11.05) | 13 | (12.15) | 1 | (5.56) | 7 | (10.61) | |
| Ethnicity | | | | | | | | | |
| Hispanic | 7 | (10.61) | 32 | (29.63) | 4 | (22.22) | 14 | (21.54) | 0.4986 |
| Non-Hispanic | 141 | (73.82) | 76 | (70.37) | 14 | (77.78) | 51 | (78.46) | |
| Body Mass Index | 34.76 | (10.17) | 30.63 | (7.54) | 41.49 | (7.45) | 39.69 | (11.40) | <0.0001 |
| [mean (SD)] | | | | | | | | | |

*Menopause status and race data were available for 190 women; ethnicity data were available for 191 women.

Global metabolic profiles of vaginal swabs: All patients provided vaginal swab and CVL samples, which were used for the global untargeted metabolomics analysis. Metabolomic profiles of CVL samples were previously described. Here, the same metabolomics platform was used to assess the metabolic profiles of patient-matched vaginal swabs and to compare them with the CVL data (see Example 1), which had been acquired in a separate UPLC-MS/MS run. 962 metabolites were identified in vaginal swabs, which was similar to the number of metabolites, 920, previously detected in CVL samples. The majority of biochemicals (84.2%) detected in vaginal swabs had known structural identity and belonged to lipid (28.2%), amino acid (21.1%), xenobiotics (14.9%), nucleotide (5.7%), peptide (5.3%), carbohydrate (4.0%), cofactors and vitamins (3.2%), energy (1.1%), and partially characterized (0.7%) superpathways. Uncharacterized metabolites were also detected and comprised 15.8% of all metabolites.

To investigate the relationship between vaginal swab metabolic profiles and the disease groups, the PLS-DA analysis was performed, which reduces the dimensionality of data to a smaller number of components for each sample. The analysis revealed significant (p=0.001) separation of samples based on the disease status, suggesting that patients with EC exhibit distinct metabolic profiles compared to patients diagnosed with benign conditions. Furthermore, to reveal patterns and connections between metabolic profiles and disease status, an unsupervised hierarchical clustering analysis was performed. The generated dendrogram and heatmap revealed two main sample clusters (data not shown). When the disease distribution was compared between these clusters, significant differences (p<0.0001) were observed. Most samples from benign controls (75%) were contained in cluster 1, whereas 68.2% of samples from women diagnosed with EC were comprised in cluster 2. These data reduction analyses showed that vaginal swab samples collected from patients with EC or benign conditions exhibit distinct metabolic profiles.

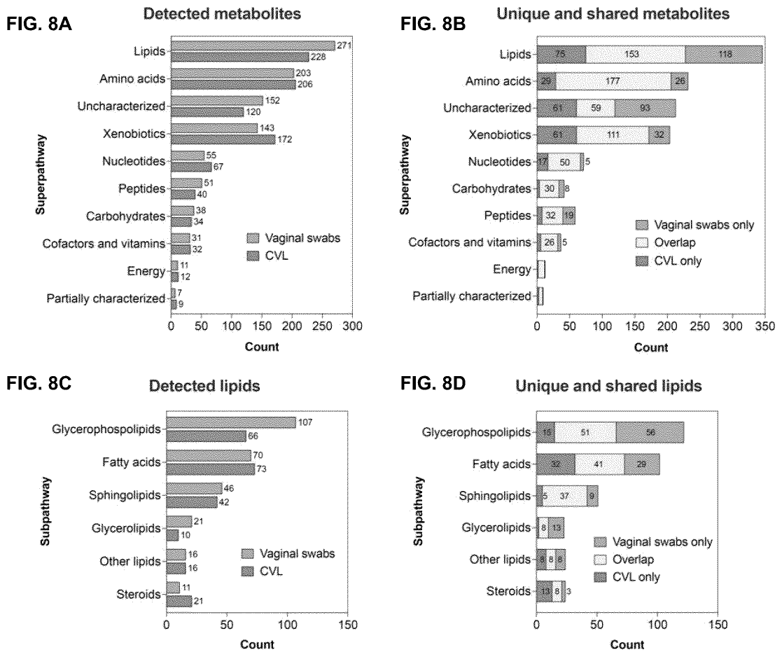

Metabolic composition of vaginal swabs and cervicovaginal lavages: Next, the metabolic composition of vaginal swabs was compared to matched and previously characterized CVL samples (Example 1). When analyzed at the metabolic superpathway level, compositions of vaginal swab and CVL samples were similar (FIG. 8A). Most of the metabolites detected in each sample type belonged to lipid, amino acid, uncharacterized, and xenobiotic superpathways. Yet, slight differences were observed in the total numbers of detected metabolites. The number of lipids was higher in vaginal swabs (271) compared to CVLs (228), whereas the number of xenobiotics was higher in CVLs (172) compared to vaginal swabs (143). The number of amino acids was similar between vaginal swabs (203) and CVL samples (206). Then, the number of unique metabolites detected only in vaginal swabs or CVLs was identified, as well as the number of overlapped metabolites detected in both sample types. Intriguingly, vaginal swab samples contained 118 unique lipid signatures, 93 unique uncharacterized metabolites, 32 unique xenobiotics, whereas CVL samples contained 75 unique lipids, 61 unique uncharacterized metabolites, and 61 unique xenobiotics (FIG. 8B). The two sample types shared 655 metabolites, mostly of amino acid (177), lipid (153), and xenobiotic (111) metabolism. Since metabolites in the lipid superpathway showed the greatest variation among sample types, lipid abundance was assessed at the subpathway level. Glycerophospholipids and glycerolipids were more prevalent in vaginal swabs, whereas CVLs had a higher number of steroids (FIG. 8C). Each sample type also exhibited a great number of unique lipids, mostly belonging to glycerophospholipid and fatty acid subpathways (FIG. 8D). These comparisons revealed that metabolic profiles of vaginal swabs substantially differ from metabolic profiles of CVL samples.

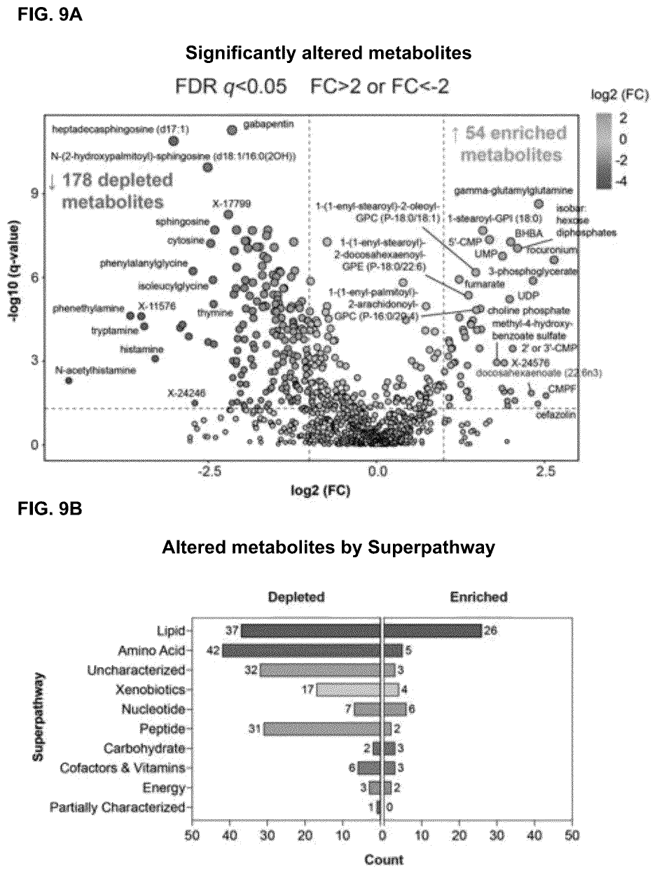

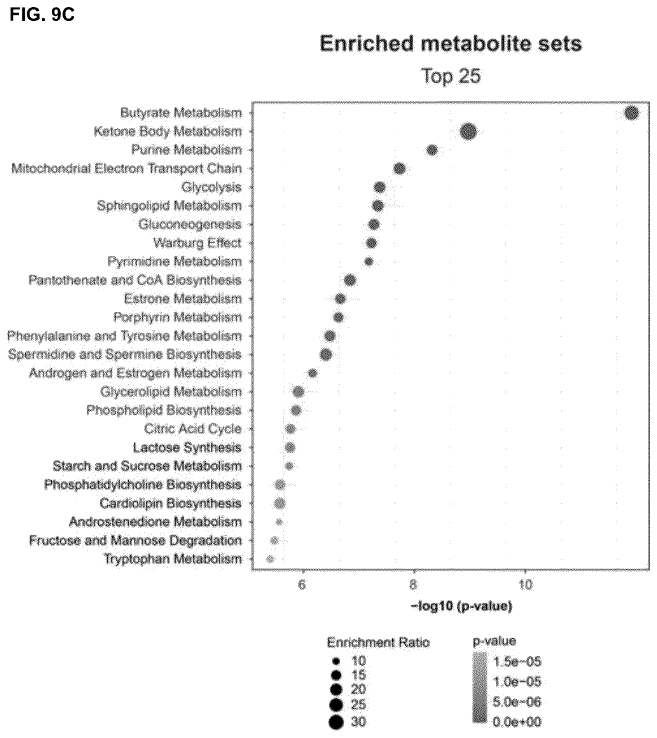

Alterations of vaginal swab metabolites in EC: To identify significantly altered metabolites in vaginal swab samples between patients diagnosed with EC and patients with benign conditions, the differential abundance analysis was performed. After false discovery correction (FDR) for multiple comparisons (q<0.05), 54 enriched and 178 depleted metabolites were identified in the EC group when compared to benign controls (FIG. 9A). When these altered metabolites were grouped based on the metabolic superpathways, the analysis revealed that most enriched metabolites were lipids (48.1%), followed by nucleotides (11.1%), whereas depleted metabolites belonged mostly to amino acid (23.6%), lipid (20.8%), uncharacterized (18.0%) and peptide (17.4%) superpathways (FIG. 9B). In addition, the quantitative metabolite set enrichment analysis identified 85 pathways to be significantly (FDR, q<0.05) altered in EC compared to benign controls. The top 25 pathways were associated mostly with lipid metabolism, amongst pathways involved in energy nucleotide, and amino acid metabolism (FIG. 9C). The most significant pathways included: butyrate metabolism (q<0.0001), ketone body metabolism (q<0.0001), purine metabolism (q<0.0001), and mitochondrial electron transport chain. Overall, these analyses revealed significant dysregulation of lipid and amino acid metabolism in EC based on the vaginal swab profiles.

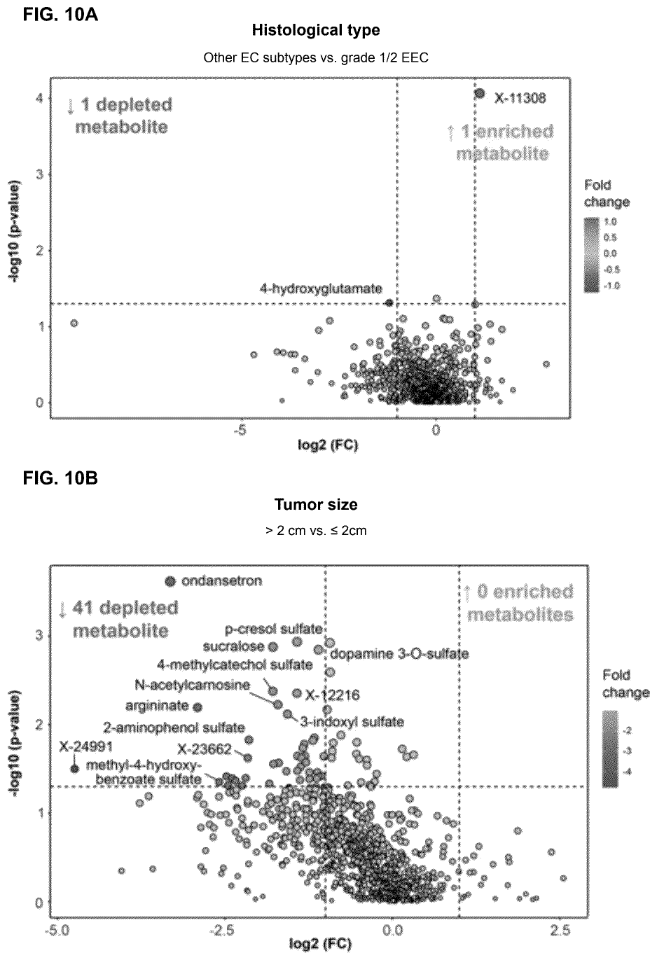

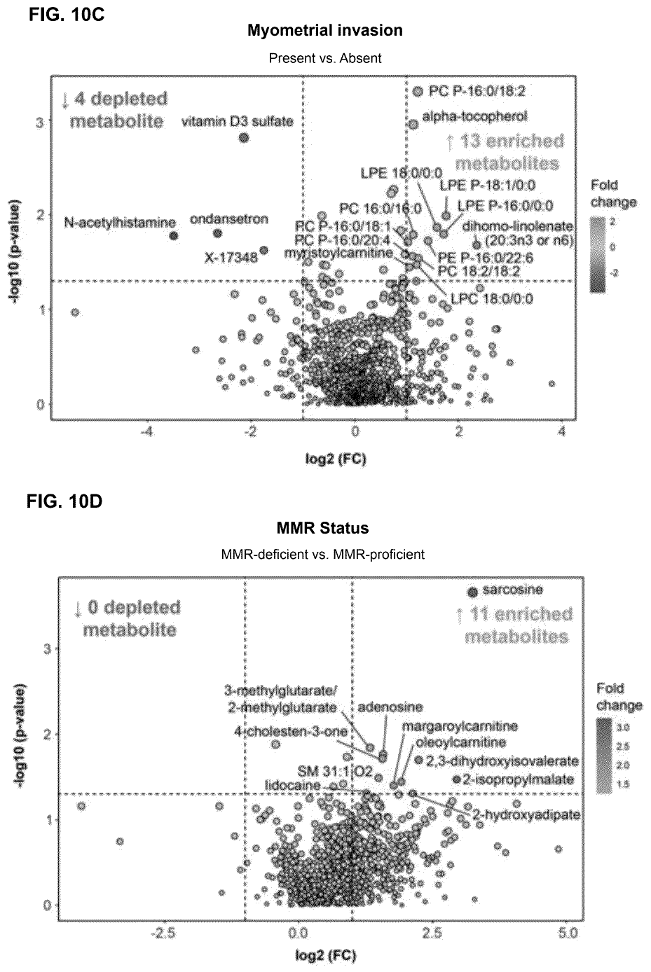

Vaginal swab metabolites and EC tumor characteristics: To evaluate potential prognostic utility of metabolites detected in vaginal swabs from patients with EC (n=66), differences in metabolite abundances were examined between patients stratified based on the tumor characteristics. Histological type and grade, tumor size, presence of myometrial invasion, and mismatch repair (MMR) protein status were analyzed (Table 7) and obtained from surgical pathology reports. Other key information on EC tumors, such as FIGO stage and presence of lymphovascular invasion, was also collected; however, the unbalanced distribution of these characteristics within the EC cohort, prevented subsequent analyses. When more aggressive EC subtypes (including grade 3 endometrioid carcinoma and serous carcinoma) was compared to grade 1 and 2 endometrial endometrioid carcinoma (EEC), one significantly (p<0.0001) enriched uncharacterized metabolite was identified, X-11308, and one significantly (p=0.0487) depleted amino acid, 4-hydroxyglutamate (FIG. 10A). Patients with larger EC tumors (>2 cm) showed depletion in 41 metabolites (p ranging from 0.0002 to 0.0489), mostly belonging to xenobiotic (16 total, including 6 metabolites of benzoate metabolism), amino acid (10 total, including four metabolites of histidine metabolism), and uncharacterized (9 metabolites) superpathways when compared to patients with smaller tumors (≤2 cm) (FIG. 10B). When myometrial invasion was present, 13 metabolites were significantly enriched, and four metabolites were significantly depleted (p ranging from 0.005 to 0.0357) in vaginal swab samples (FIG. 10C). The enriched metabolites were predominantly lipids (12 total), specifically phospholipids: four plasmalogens, two lysoplasmalogens, two lysophospholipids, and two phosphatidylcholines. Finally, samples collected from patients with MMR-proficient tumors exhibited significant (p ranging from 0.0002 to 0.0495) enrichment in 11 metabolites, with an amino acid, sarcosine, being the most elevated (FC=9.53, p=0.0002) (FIG. 10D). Yet, most enriched metabolites were lipids: four belonging to fatty acid subpathways, one sphingomyelin, and one sterol. This analysis showed that vaginal swab metabolites can be utilized to stratify patients based on EC tumor characteristics.

Table 7 shows metabolites associated with the EC tumor characteristics.

| | | |
|---|---|---|
| Metabolite Name | p-value | −log10(p-value) |

Histological Type (other EC subtypes vs. grade 1/2 EEC)

| | | |
|---|---|---|
| X-11308 | 8.63E−05 | 4.0640 |
| 4-hydroxyglutamate | 0.0487 | 1.3125 |

Tumor Size (>2 cm vs. ≤2 cm)

| | | |
|---|---|---|
| 3,7-dimethylurate | 0.0228 | 1.6414 |
| pentose acid* | 0.0381 | 1.4195 |
| N-acetylvaline | 0.0388 | 1.4112 |
| dopamine 3-O-sulfate | 0.0014 | 2.8457 |
| 1,7-dimethylurate | 0.0345 | 1.4616 |
| 1-ribosyl-imidazoleacetate* | 0.0406 | 1.3916 |
| gamma-glutamylisoleucine* | 0.0141 | 1.8509 |
| X-12830 | 0.0152 | 1.8195 |
| N-acetyltryptophan | 0.0411 | 1.3860 |
| X-15486 | 0.0345 | 1.4621 |
| hydantoin-5-propionate | 0.0266 | 1.5744 |
| 5-acetylamino-6-formylamino-3-methyluracil | 0.0226 | 1.6450 |
| N-acetyl-1-methylhistidine* | 0.0166 | 1.7794 |
| 4-methylguaiacol sulfate | 0.0184 | 1.7352 |
| X-25105 | 0.0238 | 1.6238 |
| trimethylamine N-oxide | 0.0300 | 1.5225 |
| 2R,3R-dihydroxybutyrate | 0.0423 | 1.3739 |
| 6-hydroxyindole sulfate | 0.0225 | 1.6471 |
| 5-acetylamino-6-amino-3-methyluracil | 0.0332 | 1.4784 |
| p-cresol sulfate | 0.0012 | 2.9351 |
| X-12216 | 0.0044 | 2.3550 |
| 3-indoxyl sulfate | 0.0076 | 2.1194 |
| 4-hydroxyhippurate | 0.0337 | 1.4728 |
| 3-methoxycatechol sulfate (1) | 0.0270 | 1.5682 |
| N-acetylcarnosine | 0.0060 | 2.2250 |
| phenylacetylglutamate | 0.0278 | 1.5557 |
| sucralose | 0.0013 | 2.8773 |
| 4-methylcatechol sulfate | 0.0042 | 2.3763 |
| X-17348 | 0.0463 | 1.3347 |
| 2-aminophenol sulfate | 0.0149 | 1.8274 |
| X-23662 | 0.0238 | 1.6234 |
| 1,2,3-benzenetriol sulfate (2) | 0.0401 | 1.3970 |
| 1-methylguanidine | 0.0489 | 1.3111 |
| X-25102 | 0.0431 | 1.3651 |
| doxylamine | 0.0404 | 1.3936 |
| X-17808 | 0.0484 | 1.3153 |
| histidylalanine | 0.0384 | 1.4152 |
| methyl-4-hydroxybenzoate sulfate | 0.0444 | 1.3524 |
| argininate* | 0.0064 | 2.1938 |
| ondansetron | 0.0002 | 3.6167 |
| X-24991 | 0.0317 | 1.4990 |

Myometrial Invasion (present vs. absent)

| | | |
|---|---|---|
| dihomo-linolenate (20:3n3 or n6) | 0.0210 | 1.6786 |
| 1-(1-enyl-oleoyl)-GPE (P-18:1)* | 0.0103 | 1.9870 |
| 1-(1-enyl-palmitoyl)-GPE (P-16:0)* | 0.0160 | 1.7959 |
| 1-stearoyl-GPE (18:0) | 0.0136 | 1.8670 |
| 1-(1-enyl-palmitoyl)-2-docosahexaenoyl-GPE (P-16:0/22:6)* | 0.0189 | 1.7241 |
| 1,2-dilinoleoyl-GPC (18:2/18:2) | 0.0288 | 1.5411 |
| 1-(1-enyl-palmitoyl)-2-linoleoyl-GPC (P-16:0/18:2)* | 0.0005 | 3.3004 |
| 1-stearoyl-GPC (18:0) | 0.0336 | 1.4731 |
| alpha-tocopherol | 0.0011 | 2.9523 |
| 1,2-dipalmitoyl-GPC (16:0/16:0) | 0.0163 | 1.7886 |
| 1-(1-enyl-palmitoyl)-2-arachidonoyl-GPC (P-16:0/20:4)* | 0.0274 | 1.5622 |
| myristoylcarnitine (C14) | 0.0357 | 1.4475 |
| 1-(1-enyl-palmitoyl)-2-oleoyl-GPC (P-16:0/18:1)* | 0.0194 | 1.7121 |
| X-17348 | 0.0237 | 1.6254 |
| vitamin D3 sulfate | 0.0015 | 2.8124 |
| ondansetron | 0.0157 | 1.8043 |
| N-acetylhistamine | 0.0167 | 1.7780 |

MMR Status (MMR-proficient vs MMR deficient)

| | | |
|---|---|---|
| sarcosine | 0.0002 | 3.6578 |
| 2-isopropylmalate | 0.0339 | 1.4695 |
| 2,3-dihydroxyisovalerate | 0.0201 | 1.6974 |
| 2-hydroxyadipate | 0.0495 | 1.3058 |
| oleoylcarnitine (C18:1) | 0.0361 | 1.4422 |
| margaroylcarnitine (C17)* | 0.0400 | 1.3983 |
| adenosine | 0.0173 | 1.7620 |
| 4-cholesten-3-one | 0.0194 | 1.7128 |
| sphingomyelin (d17:1/14:0, d16:1/15:0)* | 0.0325 | 1.4878 |
| 3-methylglutarate/2-methylglutarate | 0.0144 | 1.8405 |
| lidocaine | 0.0490 | 1.3102 |

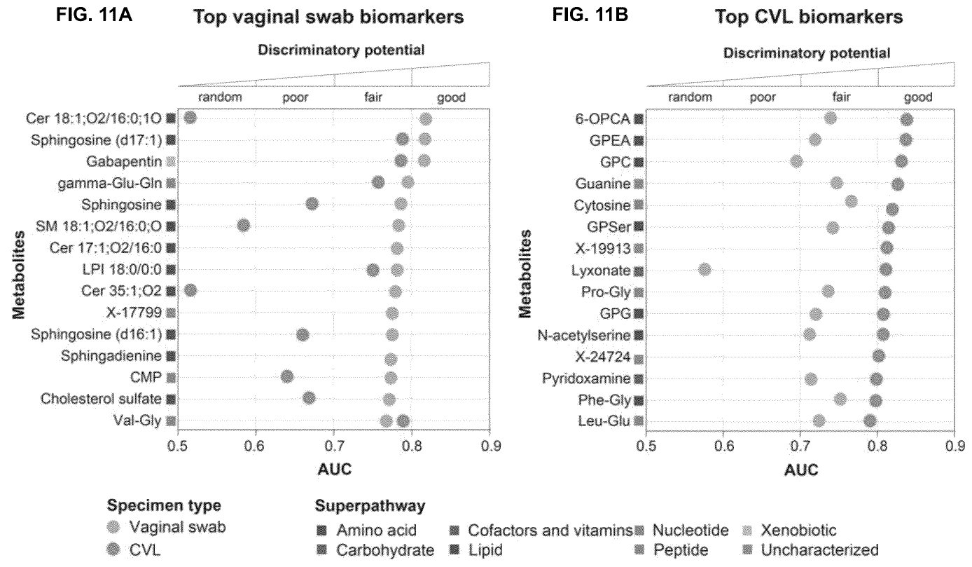

Cervicovaginal metabolic biomarkers for EC detection: To identify metabolic biomarkers with high sensitivity and specificity, a receiver operating characteristic (ROC) analysis was performed using the vaginal swab and CVL datasets. The area under the curve (AUC), which represents the overall accuracy, was used to rank the metabolites. The analysis of vaginal swab metabolites revealed three biomarkers with good ability (AUC>0.8) to discriminate the EC and benign groups, including two sphingolipids: Cer 18:1;O2/16:0;1O (AUC=0.817), sphingosine (d17:1) (AUC=0.816), and one xenobiotic, gabapentin (AUC=0.813) (FIG. 11A). Of the top 15 metabolites, 13 were also detected in CVL samples. Three of them, sphingosine (d17:1), gabapentin, and Val-Gly, had also similar AUC values ranging from 0.786 to 0.789, but none of them reached the 0.8 threshold. The analysis of CVL metabolites revealed a higher number of metabolites (12) with good discriminatory capabilities (AUC>0.8) belonging to various superpathways (FIG. 11B). The top discriminatory biomarkers included: an amino acid metabolite: 6-oxopiperidine-2-carboxylate (AUC=0.838), lipids: GPEA (AUC=0.837), GPC (AUC=0.831), and nucleotides: guanine (AUC=0.826), cytosine (AUC=0.819). The corresponding metabolites were also detected in vaginal swabs, but did not reach AUC values greater than or equal to 0.8. The ROC analyses further confirmed unique metabolic signatures detectable in vaginal swab and CVL samples. Furthermore, how the relationships were evaluated between vaginal swab metabolites and the disease groups change when accounting for age and BMI, since these factors significantly differ between patients diagnosed with EC or benign conditions (Table 6). The analysis showed that the observed difference in levels of key vaginal swab metabolites between the groups was not diminished after the age and BMI adjustment, similarly as previously observed for the CVL data (Example 1).

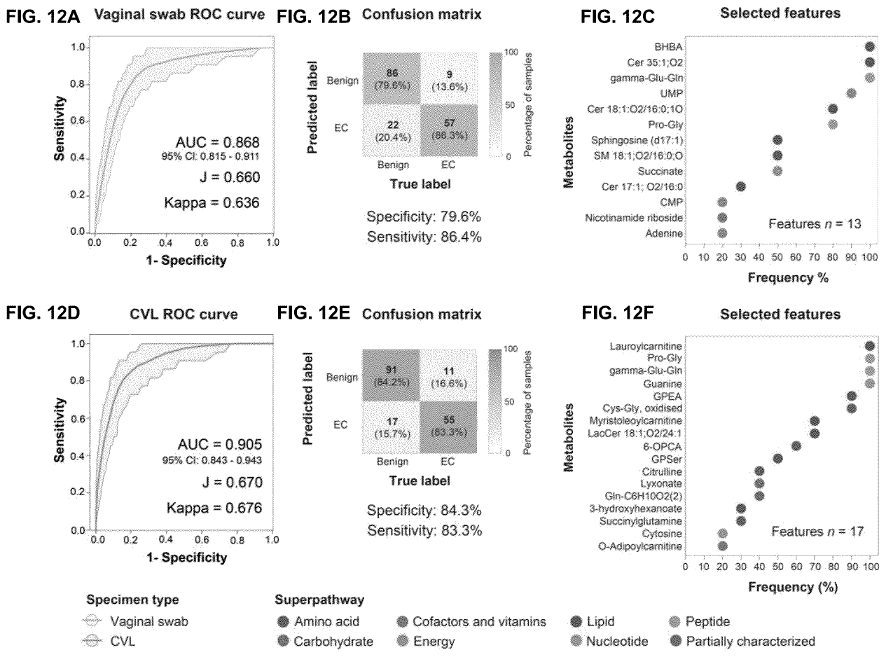

Multivariable vaginal swab and CVL models to predict EC: To evaluate the ability of cervicovaginal metabolites detected in vaginal swab and CVL samples to predict EC and benign conditions, we used multivariable logistic regression models. The predictive models were built using a vaginal swab or CVL dataset and the feature selection was based on the LASSO algorithm (FIG. 12A-12F). Models with various numbers of features were empirically tested. The best-performing model utilizing the vaginal swab data consisted of 13 features and demonstrated a good discriminatory ability to predict EC and benign conditions (AUC 0.868) (FIG. 12A). This model correctly identified 86 out 108 patients with benign conditions (specificity of 79.6%) and 57 out of 66 patients with EC (sensitivity of 86.4%) (FIG. 12B). The features selected for the vaginal swab model included mostly metabolites belonging to lipid (six features), nucleotide (three features) and peptide (two features) superpathways, in addition to single features belonging to energy and cofactors and vitamins superpathways (FIG. 12C). The best-performing model utilizing the CVL data consisted of 17 features and demonstrated an excellent ability to discriminate patients with EC and benign conditions (AUC 0.905) (FIG. 12D). This model correctly predicted 91 out 108 patients with benign conditions (specificity of 84.3%) and 55 out of 66 patients with EC (sensitivity of 83.3%) (FIG. 12E). The features selected for the CVL model included a diverse array of metabolites belonging to lipid (six features), amino acid (four features), nucleotide (three features), and peptide (two features) superpathways, in addition to single features belonging to carbohydrate, cofactors and vitamins, and partially characterized superpathways (FIG. 12F). When compared to the vaginal swab model, the CVL model exhibited marginally better overall performance indicated by Youden's index (0.670 vs. 0.660) and inter-rater reliability (or degree of agreement) indicated by Cohen's kappa (0.676 vs. 0.636), respectively. Yet, the vaginal swab model consisted of less features and exhibited slightly higher sensitivity compared to the CVL model. Although in both models, lipids were the most selected features (35-46%), the models utilized different lipid signatures and only two peptide features (γ-Glu-Gln and Pro-Gly) were shared by the two models. Overall, the performance metrics of the vaginal swab and CVL models were comparable, but the models utilized different combinations of metabolites for the most accurate prediction of the disease groups.

### Embodiments

The following embodiments are intended to be illustrative only and not to be limiting in any way.

Embodiment 1: A method comprising: a) obtaining a cervicovaginal lavage (CVL) sample from a patient; b) producing a profile of the CVL sample collected in (a) by detecting at least five or more metabolite biomarkers selected from one or a combination of: 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine; and c) measuring the CVL sample profile produced in (b). In some embodiments, the profile of the CVL sample collected in (a) is produced by detecting at least five or more metabolite biomarkers selected from the group consisting of (or consisting essentially of) 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine. Embodiment 2: The method of embodiment 1, wherein producing a profile comprises detecting at least ten or more biomarkers. Embodiment 3: The method of embodiment 1 or embodiment 2, wherein producing a profile comprises detecting at least 15 or more biomarkers. Embodiment 4: The method of any one of embodiments 1-3, wherein producing a profile comprises detecting at least 20 or more biomarkers. Embodiment 5: The method of any one of embodiments 1-4, wherein the metabolite biomarkers are expressed in grade ½ endometrioid endometrial cancer (EEC). Embodiment 6: The method of any one of embodiments 1-5, wherein the metabolite biomarkers further comprise one or more of AMP, spermine, myristoleoylcarnitine, heptadecasphingosine, myristoylcarnitine, pryridoxamine, 3-hydroxyhexanoate, GPC (16:0/20:3), palmitoleoylcamitine. Embodiment 7: The method of embodiment 6, wherein the metabolite biomarkers are expressed in all endometrial cancer (EMC). Embodiment 8: The method of any one of embodiments 1-7, wherein the metabolite biomarkers further comprise one or more of biliverdin, PC (P-16:0/20:4), 7-HOCA, PC (P-16:0/16:0), BHBA, X-25004, glycolithocholate sulfate, N-acetylserine, 3-hydroxyhexanoate, myristolycarnitine (C14:1), X-19913. Embodiment 9: The method of embodiment 8, wherein the metabolite biomarkers are expressed in aggressive forms of endometrial cancer (EMC). Embodiment 10: The method of any one of embodiments 1-9, wherein the method predicts the risk of endometrial hyperplasia or cancer in women. Embodiment 11: The method of any one of embodiments 1-10, wherein the method diagnoses endometrial cancer in women. Embodiment 12: The method of any one of embodiments 1-11, wherein the endometrial cancer is EC type 1.

Embodiment 13: A method comprising: a) obtaining a vaginal swab sample from a patient; b) producing a profile of the vaginal swab sample collected in (a) by detecting at least five or more metabolite biomarkers selected from one or more of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP); c) analyzing the vaginal swab sample profile produced in (b). In some embodiments, the profile of the CVL sample collected in (a) is produced by detecting at least five or more metabolite biomarkers selected from the group consisting of (or consisting essentially of) N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP). Embodiment 14: The method of embodiment 13, wherein producing a profile comprises detecting at least ten or more biomarkers. Embodiment 15: The method of embodiment 13 or embodiment 14, wherein producing a profile comprises detecting at least 15 or more biomarkers. Embodiment 16: The method of any one of embodiments 13-15, wherein producing a profile comprises detecting at least 20 or more biomarkers. Embodiment 17: The method of any one of embodiments 13-16, wherein the metabolite biomarkers are expressed in grade ½ endometrioid endometrial cancer (EEC). Embodiment 18: The method of any one of embodiments 13-17, wherein the method predicts the risk of endometrial hyperplasia or cancer in women. Embodiment 19: The method of any one of embodiments 13-18, wherein the method diagnoses endometrial cancer in women. Embodiment 20: The method of any one of embodiments 13-19, wherein the endometrial cancer is EC type 1.

Embodiment 21: A non-invasive method of diagnosing endometrial cancer (EC) in a subject in need thereof, the method comprising: a) determining the subject's levels of five or more metabolite biomarkers by: i) obtaining a cervicovaginal lavage (CVL) sample from the patient; and ii) measuring the levels of at least five or more metabolite biomarkers in the sample obtained in (i); wherein the metabolite biomarkers are selected from one or a combination of: 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine; and b) diagnosing the subject with EC if the levels of at least five biomarkers are altered compared to a control profile. In some embodiments, the method comprises an in vitro method of diagnosing endometrial cancer (EC) in a subject in need thereof, the method comprising: a) producing a profile from a CVL sample having been obtained from a subject by detecting at least five or more metabolite biomarkers selected from one or a combination of: 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine; and b) diagnosing the patient with EC if the levels of at least five biomarkers are altered compared to a control profile. In some embodiments, the the metabolite biomarkers are selected from the group consisting of (or consisting essentially of): 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine. Embodiment 22: The method of embodiment 21, wherein the method comprises measuring the levels of at least ten or more metabolite biomarkers in the sample obtained. Embodiment 23: The method of embodiment 21 or embodiment 22, wherein the method comprises measuring the levels of at least 15 or more metabolite biomarkers in the sample obtained. Embodiment 24: The method of any one of embodiments 21-23, wherein the method comprises measuring the levels of at least 20 or more metabolite biomarkers in the sample obtained. Embodiment 25: The method of any one of embodiments 21-24, wherein the subject is diagnosed with EC when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0), hexadecasphingosine (d16:1), X-17799, sphingadienine, or cholesterol sulfate are downregulated compared to a control profile and gamma-glutamylglutamine, 1-stearoyl-GPI (18:0), or cytidine 5′-monophosphate (5′-CMP) are upregulated compared to a control profile. Embodiment 26: The method of any one of embodiments 21-25, wherein the metabolite biomarkers are expressed in grade ½ endometrioid endometrial cancer (EEC). Embodiment 27: The method of any one of embodiments 21-26, wherein the metabolite biomarkers further comprise one or more of AMP, spermine, myristoleoylcamitine, heptadecasphingosine, myristoylcarnitine, pryridoxamine, 3-hydroxyhexanoate, GPC (16:0/20:3), palmitoleoylcarnitine. Embodiment 28: The method of embodiment 27, wherein the metabolite biomarkers are expressed in all endometrial cancer (EMC). Embodiment 29: The method of any one of embodiments 21-28, wherein the metabolite biomarkers further comprise one or more of biliverdin, PC (P-16:0/20:4), 7-HOCA, PC (P-16:0/16:0), BHBA, X-25004, glycolithocholate sulfate, N-acetylserine, 3-hydroxyhexanoate, myristolycarnitine (C14:1), X-19913. Embodiment 30: The method of embodiment 29, wherein the metabolite biomarkers are expressed in aggressive forms of endometrial cancer (EMC). Embodiment 31: The method of any one of embodiments 21-30, wherein the method predicts the risk of endometrial hyperplasia or cancer in women. Embodiment 32: The method of any one of embodiments 21-31, wherein the method diagnoses endometrial cancer in women. Embodiment 33: The method of any one of embodiments 21-32, wherein the endometrial cancer is EC type 1. Embodiment 34: The method of any one of embodiments 21-33, further comprising administering a therapeutic amount of a treatment to the patient if the patient is diagnosed with endometrial cancer (EC).

Embodiment 35: A non-invasive method of diagnosing endometrial cancer (EC) in a subject in need thereof, the method comprising: a) determining the subject's levels of five or more metabolite biomarkers by: i) obtaining a vaginal swab sample from the subject; and ii) measuring the levels of at least five or more metabolite biomarkers in the sample obtained in (i); wherein the metabolite biomarkers are selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP); and b) diagnosing the subject with EC if the levels of at least five biomarkers are altered compared to a control profile. In some embodiments, the method comprises an in vitro method of diagnosing endometrial cancer (EC) in a subject in need thereof, the method comprising: a) producing a profile from a vaginal swab sample having been obtained from a subject by detecting at least five or more metabolite biomarkers selected from one or a combination of: 6-oxopiperidine-2-carboxylate, GPEA, GPC, guanine, cytosine, glycerophosphoserine, X-19913, X-24724 lyxonate, prolylglycine, glycerophosphoglycerol, or N-acetylserine; and b) diagnosing the patient with EC if the levels of at least five biomarkers are altered compared to a control profile. In some embodiments, the metabolite biomarkers are selected from the group consisting of (or consisting essentially of): N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP). Embodiment 36: The method of embodiment 35, wherein the method comprises measuring the levels of at least ten or more metabolite biomarkers in the sample obtained. Embodiment 37: The method of embodiment 35 or embodiment 36, wherein the method comprises measuring the levels of at least 15 or more metabolite biomarkers in the sample obtained. Embodiment 38: The method of any one of embodiments 35-37, wherein the method comprises measuring the levels of at least 20 or more metabolite biomarkers in the sample obtained. Embodiment 39: The method of any one of embodiments 35-38, wherein the subject is diagnosed with EC with cancer when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are downregulated and gamma-glutamylglutamine, 1-stearoyl-GPI (18:0), or cytidine 5′-monophosphate (5′-CMP) are upregulated. Embodiment 40: The method of any one of embodiments 35-39, wherein the metabolite biomarkers are expressed in grade ½ endometrioid endometrial cancer (EEC).

Embodiment 41: A method of treating endometrial cancer (EC) in a patient in need thereof, the method comprising: a) diagnosing endometrial cancer (EC) in the patient by: i) obtaining a cervicovaginal lavage (CVL) sample from the patient; ii) measuring the levels of at least five or more metabolite biomarkers in the sample obtained in (i); wherein the metabolite biomarkers are selected from one or a combination of: 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine; b) diagnosing the patient with EC if the levels of at least five biomarkers in the profile obtained in (ii) are altered compared to a control profile; and c) administering a therapeutic amount of a treatment to the patient if the patient is diagnosed with EC. In some embodiments, the metabolite biomarkers are selected from the group consisting of (or consisting essentially of) 6-oxopiperidine-2-carboxylate, GPEA, GPC, guanine, cytosine, glycerophosphoserine, X-19913, X-24724 lyxonate, prolylglycine, glycerophosphoglycerol, or N-acetylserine Embodiment 42: The method of embodiment 41, wherein the method comprises measuring the levels of at least ten or more metabolite biomarkers in the sample obtained. Embodiment 43: The method of embodiment 41 or embodiment 42, wherein the method comprises measuring the levels of at least 15 or more metabolite biomarkers in the sample obtained. Embodiment 44: The method of any one of embodiments 41-43, wherein the method comprises measuring the levels of at least 20 or more metabolite biomarkers in the sample obtained. Embodiment 45: The method of any one of embodiments 41-44, wherein the subject is diagnosed with EC when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are downregulated compared to the control profile and gamma-glutamylglutamine, 1-stearoyl-GPI (18:0), or cytidine 5′-monophosphate (5′-CMP) are upregulated compared to the control profile. Embodiment 46: The method of any one of embodiments 41-45, wherein the metabolite biomarkers are expressed in grade ½ endometrioid endometrial cancer (EEC). Embodiment 47: The method of any one of embodiments 41-46, wherein the metabolite biomarkers further comprise one or more of AMP, spermine, myristoleoylcarnitine, heptadecasphingosine, myristoylcarnitine, pryridoxamine, 3-hydroxyhexanoate, GPC (16:0/20:3), palmitoleoylcarnitine. Embodiment 48: The method of embodiment 47, wherein the metabolite biomarkers are expressed in all endometrial cancer (EMC). Embodiment 49: The method of any one of embodiments 41-48, wherein the metabolite biomarkers further comprise one or more of biliverdin, PC (P-16:0/20:4), 7-HOCA, PC (P-16:0/16:0), BHBA, X-25004, glycolithocholate sulfate, N-acetylserine, 3-hydroxyhexanoate, myristolycarnitine (C14:1), X-19913. Embodiment 50: The method of embodiment 49, wherein the metabolite biomarkers are expressed in aggressive forms of endometrial cancer (EMC). Embodiment 51: The method of any one of embodiments 41-50, wherein the method predicts the risk of endometrial hyperplasia or cancer in women. Embodiment 52: The method of any one of embodiments 41-51, wherein the method diagnoses endometrial cancer in women. Embodiment 53: The method of any one of embodiments 41-52, wherein the endometrial cancer is EC type 1.

Embodiment 54: The method of any one of embodiments 41-52, wherein producing a profile further comprises characterizing endometrial tumor characteristics comprising tumor size, myometrial invasion, mismatch repair (MMR) status, histological grade, age, or a combination thereof. Embodiment 55: The method of embodiment 54, wherein tumor size is determined by metabolite biomarkers selected from a group comprising dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, and cytosine. Embodiment 56: The method of embodiment 55, wherein dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) are upregulated in tumors greater than or equal to 2 cm and CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, cytosine are down-regulated in tumors greater than or equal to 2 cm. Embodiment 57: The method of embodiment 54, wherein myometrial invasion is determined by metabolite biomarkers selected from a group comprising ceramide (d18:1/14:0, d16:1/16:0)*, (3′-5′)-cytidylyluridine*, N-stearoyl-sphingosine (d18:1/18:0)*, N-palmitoyl-sphinganine (d18:0/16:0), N-palmitoyl-sphingosine (d18:1/16:0), 1-(1-enyl-palmitoyl)-2-palmitoyl-GPC (P-16:0/16:0)*, alpha-hydroxyisocaproate, CMP, 2′-AMP, AMP, argininate*, 2,3-diphosphoglycerate, cyclic adenosine diphosphate-ribose, histamine, and tryptamine. Embodiment 58: The method of embodiment 57, wherein ceramide (d18:1/14:0, d16:1/16:0)*, (3′-5′)-cytidylyluridine*, N-stearoyl-sphingosine (d18:1/18:0)*, N-palmitoyl-sphinganine (d18:0/16:0), N-palmitoyl-sphingosine (d18:1/16:0), and 1-(1-enyl-palmitoyl)-2-palmitoyl-GPC (P-16:0/16:0)* are upregulated in the in the absence of myometrial invasion and alpha-hydroxyisocaproate, CMP, 2′-AMP, AMP, argininate*, 2,3-diphosphoglycerate, cyclic adenosine diphosphate-ribose, histamine, and tryptamine are down-regulated in the absence of myometrial invasion. Embodiment 59: The method of embodiment 54, wherein mismatch repair (MMR) status is determined by metabolite biomarkers selected from a group comprising decanoylcarnitine (C10), octanoylcarnitine (C8), laurylcarnitine (C12), glutarate (C5-DC), S-adenosylmethionine (SAM), butyrylcarnitine (C4), lyxonate, 6-oxopiperidine-2-carboxylate, adenosine, guanine, and sarcosine. Embodiment 60: The method of embodiment 59, wherein decanoylcarnitine (C10), octanoylcarnitine (C8), and laurylcarnitine (C12) are upregulated in MMR-proficient cancer and glutarate (C5-DC), S-adenosylmethionine (SAM), butyrylcarnitine (C4), lyxonate, 6-oxopiperidine-2-carboxylate, adenosine, guanine, and sarcosine are down-regulated in MMR deficient cancer. Embodiment 61: The method of embodiment 54, wherein the histological grade is determined by metabolite biomarkers selected from a group comprising 1-oleoyl-GPS (18:1), pregnen-diol disulfate, dehydroepiandrosterone sulfate (DHEA-S), kynurenine, 1-(1-enyl-palmitoyl)-2-arachidonoyl-GPC (P-16:0/20:4), pregnenetriol disulfate, 1-stearoyl-2-docosahexaenoyl-GPC (18:0/22:6), 1,2-dilinoleoyl-GPC (18:2/18:2), 1-palmitoyl-2-palmitoleoyl-GPC (16:0/16:1), 1-linoleoyl-2-arachidonoyl-GPC (18:2/20:4n6), androsterone sulfate, 1-palmitoyl-2-arachidonoyl-GPC (16:0/20:4n6), androstenediol (3beta,17beta) disulfate (2), 1-stearoyl-2-linoleoyl-GPC (18:0/18:2), 1-palmitoyl-2-docosahexaenoyl-GPC (16:0/22:6), 1-stearoyl-2-arachidonoyl-GPC (18:0/20:4), N-methylhydroxyproline, 3-hydroxybutyrate (BHBA), sphingomyelin (d18:2/16:0, d18:1/16:1), 1-palmitoyl-2-dihomo-linolenoyl-GPC (16:0/20:3n3 or 6), 1-palmitoyl-2-linoleoyl-GPC (16:0/18:2), sphingomyelin (d18:1/20:1, d18:2/20:0), sphingomyelin (d18:1/18:1, d18:2/18:0), 1-(1-enyl-palmitoyl)-2-linoleoyl-GPC (P-16:0/18:2), 1-oleoyl-2-linoleoyl-GPC (18:1/18:2), 2-methylbutyrylcarnitine (C5), and homocysteine. Embodiment 62: The method of embodiment 61, wherein 1-oleoyl-GPS (18:1), pregnen-diol disulfate, dehydroepiandrosterone sulfate (DHEA-S), kynurenine, 1-(1-enyl-palmitoyl)-2-arachidonoyl-GPC (P-16:0/20:4), pregnenetriol disulfate, 1-stearoyl-2-docosahexaenoyl-GPC (18:0/22:6), 1,2-dilinoleoyl-GPC (18:2/18:2), 1-palmitoyl-2-palmitoleoyl-GPC (16:0/16:1), 1-linoleoyl-2-arachidonoyl-GPC (18:2/20:4n6), androsterone sulfate, 1-palmitoyl-2-arachidonoyl-GPC (16:0/20:4n6), androstenediol (3beta,17beta) disulfate (2), 1-stearoyl-2-linoleoyl-GPC (18:0/18:2), 1-palmitoyl-2-docosahexaenoyl-GPC (16:0/22:6), 1-stearoyl-2-arachidonoyl-GPC (18:0/20:4), N-methylhydroxyproline, 3-hydroxybutyrate (BHBA), sphingomyelin (d18:2/16:0, d18:1/16:1), 1-palmitoyl-2-dihomo-linolenoyl-GPC (16:0/20:3n3 or 6), 1-palmitoyl-2-linoleoyl-GPC (16:0/18:2), sphingomyelin (d18:1/20:1, d18:2/20:0), sphingomyelin (d18:1/18:1, d18:2/18:0), 1-(1-enyl-palmitoyl)-2-linoleoyl-GPC (P-16:0/18:2), and 1-oleoyl-2-linoleoyl-GPC (18:1/18:2) are upregulated in ½ EEC and 2-methylbutyrylcarnitine (C5) and homocysteine are down-regulated in ½ EEC. Embodiment 63: The method of embodiment 54, wherein the age is determined by metabolite biomarkers selected from a group comprising N6-methyllysine, nicotinate ribonucleoside, or a combination thereof.

Embodiment 64: A method of treating endometrial cancer (EC) in a patient in need thereof, the method comprising: a) diagnosing endometrial cancer (EC) in the patient by: i) obtaining a vaginal swab sample from the patient; ii) measuring the levels of at least five or more metabolite biomarkers in the sample obtained in (i); wherein the metabolite biomarkers are selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP); and iii) diagnosing the patient with EC if the levels of at least five biomarkers are altered compared to a control profile and b) administering a therapeutic amount of a treatment to the patient if the patient is diagnosed with EC. Embodiment 65: The method of embodiment 64, wherein the method comprises measuring the levels of at least ten or more metabolite biomarkers in the sample obtained. Embodiment 66: The method of embodiment 64 or embodiment 65, wherein the method comprises measuring the levels of at least 15 or more metabolite biomarkers in the sample obtained. Embodiment 67: The method of any one of embodiments 64-66, wherein the method comprises measuring the levels of at least 20 or more metabolite biomarkers in the sample obtained. Embodiment 68: The method of any one of embodiments 64-67, wherein the method diagnoses endometrial cancer (EC) in the subject, wherein a subject is diagnosed with EC with cancer when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are downregulated and gamma-glutamylglutamine, 1-stearoyl-GPI (18:0), or cytidine 5′-monophosphate (5′-CMP) are upregulated compared to a control profile. Embodiment 69: The method of any one of embodiments 64-68, wherein the metabolite biomarkers are expressed in grade ½ endometrioid endometrial cancer (EEC).

Embodiment 70: The method of any one of embodiments 64-69 wherein producing a profile further comprises characterizing endometrial tumor characteristics comprising tumor size, myometrial invasion, mismatch repair (MMR) status, histological grade, age, or a combination thereof. Embodiment 71: The method of embodiment 70, wherein tumor size is determined by metabolite biomarkers selected from a group comprising 3,7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991. Embodiment 72: The method of embodiment 71, wherein 3,7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991 are down-regulated in tumors greater than or equal to 2 cm. Embodiment 73: The method of embodiment 54, wherein myometrial invasion is determined by metabolite biomarkers selected from a group comprising dihomo-linolenate (20:3n3 or n6), 1-(1-enyl-oleoyl)-GPE (P-18:1)*, 1-(1-enyl-palmitoyl)-GPE (P-16:0)*, 1-stearoyl-GPE (18:0), 1-(1-enyl-palmitoyl)-2-docosahexaenoyl-GPE (P-16:0/22:6)*, 1,2-dilinoleoyl-GPC (18:2/18:2), 1-(1-enyl-palmitoyl)-2-linoleoyl-GPC (P-16:0/18:2)*, 1-stearoyl-GPC (18:0), alpha-tocopherol, 1,2-dipalmitoyl-GPC (16:0/16:0), 1-(1-enyl-palmitoyl)-2-arachidonoyl-GPC (P-16:0/20:4)*, myristoylcarnitine (C14), 1-(1-enyl-palmitoyl)-2-oleoyl-GPC (P-16:0/18:1)*, X-17348, vitamin D3 sulfate, ondansetron, and N-acetylhistamine. Embodiment 74: The method of embodiment 73, wherein dihomo-linolenate (20:3n3 or n6), 1-(1-enyl-oleoyl)-GPE (P-18:1)*, 1-(1-enyl-palmitoyl)-GPE (P-16:0)*, 1-stearoyl-GPE (18:0), 1-(1-enyl-palmitoyl)-2-docosahexaenoyl-GPE (P-16:0/22:6)*, 1,2-dilinoleoyl-GPC (18:2/18:2), 1-(1-enyl-palmitoyl)-2-linoleoyl-GPC (P-16:0/18:2)*, 1-stearoyl-GPC (18:0), alpha-tocopherol, 1,2-dipalmitoyl-GPC (16:0/16:0), 1-(1-enyl-palmitoyl)-2-arachidonoyl-GPC (P-16:0/20:4)*, myristoylcarnitine (C14), 1-(1-enyl-palmitoyl)-2-oleoyl-GPC (P-16:0/18:1)* are upregulated in the absence of myometrial invasion and X-17348, vitamin D3 sulfate, ondansetron, and N-acetylhistamine are down-regulated in the absence of myometrial invasion. Embodiment 75: The method of embodiment 54, wherein mismatch repair (MMR) status is determined by metabolite biomarkers selected from a group comprising lidocaine, 3-methylglutarate/2-methylglutarate, sphingomyelin (d17:1/14:0, d16:1/15:0)*, 4-cholesten-3-one, adenosine, margaroylcarnitine (C17)*, oleoylcarnitine (C18:1), 2-hydroxyadipate, 2,3-dihydroxyisovalerate, 2-isopropylmalate, and sarcosine. Embodiment 76: The method of embodiment 75, wherein are lidocaine, 3-methylglutarate/2-methylglutarate, sphingomyelin (d17:1/14:0, d16:1/15:0)*, 4-cholesten-3-one, adenosine, margaroylcarnitine (C17)*, oleoylcarnitine (C18:1), 2-hydroxyadipate, 2,3-dihydroxyisovalerate, 2-isopropylmalate, and sarcosine down-regulated in MMR proficient cancer. Embodiment 77: The method of embodiment 70, wherein the histological grade is determined by metabolite biomarkers selected from a group comprising X-11308 or 4-hydroxyglutamate. Embodiment 78: The method of embodiment 77, wherein X-11308 is upregulated in ½ EEC and 4-hydroxyglutamate is down-regulated in ½ EEC.

Embodiment 79: A method of monitoring a treatment for endometrial cancer (EC) in a subject in need thereof, the method comprising; a) obtaining a first cervicovaginal lavage (CVL) sample from the subject; b) producing a baseline profile of the CVL sample collected in (a) by detecting at least five or more metabolite biomarkers in the sample obtained in (a); wherein the metabolite biomarkers are selected from one or a combination of: 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine; c) administering the treatment for EC to the subject; d) obtaining a second cervicovaginal lavage (CVL) sample from the subject; e) producing a second profile of the CVL sample collected in (d) by detecting at least at least five or more metabolite biomarkers in the sample obtained in (d); wherein the metabolite biomarkers are selected from one or a combination of: 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine; and f) comparing the baseline profile of the CVL sample produced in (b) to the second profile of the CVL sample produced in (e); wherein the treatment is effective if the levels of at least five biomarkers are altered from the baseline profile as compared to the second profile. Embodiment 80: The method of embodiment 79, wherein the method comprises measuring the levels of at least ten or more metabolite biomarkers in the sample obtained. Embodiment 81: The method of embodiment 79 or embodiment 80, wherein the method comprises measuring the levels of at least 15 or more metabolite biomarkers in the sample obtained. Embodiment 82: The method of any one of embodiments 79-81, wherein the method comprises measuring the levels of at least 20 or more metabolite biomarkers in the sample obtained. Embodiment 83: The method of any one of embodiments 79-82, wherein the treatment is effective when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are upregulated and gamma-glutamylglutamine, 1-stearoyl-GPI (18:0), or cytidine 5′-monophosphate (5′-CMP) are down-regulated. Embodiment 84: The method of any one of embodiments 79-83, wherein the metabolite biomarkers are expressed in grade ½ endometrioid endometrial cancer (EEC). Embodiment 85: The method of any one of embodiments 79-84, wherein the metabolite biomarkers further comprise one or more of AMP, spermine, myristoleoylcarnitine, heptadecasphingosine, myristoylcarnitine, pryridoxamine, 3-hydroxyhexanoate, GPC (16:0/20:3), palmitoleoylcarnitine. Embodiment 86: The method of embodiment 85, wherein the metabolite biomarkers are expressed in all endometrial cancer (EMC). Embodiment 87: The method of any one of embodiments 79-86, wherein the metabolite biomarkers further comprise one or more of biliverdin, PC (P-16:0/20:4), 7-HOCA, PC (P-16:0/16:0), BHBA, X-25004, glycolithocholate sulfate, N-acetylserine, 3-hydroxyhexanoate, myristolycarnitine (C14:1), X-19913.

Embodiment 88: The method of any one of embodiments 79-87, wherein producing a profile further comprises characterizing endometrial tumor characteristics comprising tumor size, myometrial invasion, mismatch repair (MMR) status, histological grade, age, or a combination thereof. Embodiment 89: The method of embodiment 88, wherein tumor size is determined by metabolite biomarkers selected from a group comprising dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, and cytosine. Embodiment 90: The method of embodiment 89, wherein dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) are upregulated in tumors greater than or equal to 2 cm and CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, cytosine are down-regulated in tumors greater than or equal to 2 cm. Embodiment 91: The method of embodiment 88, wherein myometrial invasion is determined by metabolite biomarkers selected from a group comprising ceramide (d18:1/14:0, d16:1/16:0)*, (3′-5′)-cytidylyluridine*, N-stearoyl-sphingosine (d18:1/18:0)*, N-palmitoyl-sphinganine (d18:0/16:0), N-palmitoyl-sphingosine (d18:1/16:0), 1-(1-enyl-palmitoyl)-2-palmitoyl-GPC (P-16:0/16:0)*, alpha-hydroxyisocaproate, CMP, 2′-AMP, AMP, argininate*, 2,3-diphosphoglycerate, cyclic adenosine diphosphate-ribose, histamine, and tryptamine. Embodiment 92: The method of embodiment 91, wherein ceramide (d18:1/14:0, d16:1/16:0)*, (3′-5′)-cytidylyluridine*, N-stearoyl-sphingosine (d18:1/18:0)*, N-palmitoyl-sphinganine (d18:0/16:0), N-palmitoyl-sphingosine (d18:1/16:0), and 1-(1-enyl-palmitoyl)-2-palmitoyl-GPC (P-16:0/16:0)* are upregulated in the in the absence of myometrial invasion and alpha-hydroxyisocaproate, CMP, 2′-AMP, AMP, argininate*, 2,3-diphosphoglycerate, cyclic adenosine diphosphate-ribose, histamine, and tryptamine are down-regulated in the absence of myometrial invasion. Embodiment 93: The method of embodiment 88, wherein mismatch repair (MMR) status is determined by metabolite biomarkers selected from a group comprising decanoylcarnitine (C10), octanoylcarnitine (C8), laurylcarnitine (C12), glutarate (C5-DC), S-adenosylmethionine (SAM), butyrylcarnitine (C4), lyxonate, 6-oxopiperidine-2-carboxylate, adenosine, guanine, and sarcosine. Embodiment 94: The method of embodiment 93, wherein decanoylcarnitine (C10), octanoylcarnitine (C8), and laurylcarnitine (C12) are upregulated in MMR-proficient cancer and glutarate (C5-DC), S-adenosylmethionine (SAM), butyrylcarnitine (C4), lyxonate, 6-oxopiperidine-2-carboxylate, adenosine, guanine, and sarcosine are down-regulated in MMR deficient cancer. Embodiment 95: The method of embodiment 88, wherein the histological grade is determined by metabolite biomarkers selected from a group comprising 1-oleoyl-GPS (18:1), pregnen-diol disulfate, dehydroepiandrosterone sulfate (DHEA-S), kynurenine, 1-(1-enyl-palmitoyl)-2-arachidonoyl-GPC (P-16:0/20:4), pregnenetriol disulfate, 1-stearoyl-2-docosahexaenoyl-GPC (18:0/22:6), 1,2-dilinoleoyl-GPC (18:2/18:2), 1-palmitoyl-2-palmitoleoyl-GPC (16:0/16:1), 1-linoleoyl-2-arachidonoyl-GPC (18:2/20:4n6), androsterone sulfate, 1-palmitoyl-2-arachidonoyl-GPC (16:0/20:4n6), androstenediol (3beta,17beta) disulfate (2), 1-stearoyl-2-linoleoyl-GPC (18:0/18:2), 1-palmitoyl-2-docosahexaenoyl-GPC (16:0/22:6), 1-stearoyl-2-arachidonoyl-GPC (18:0/20:4), N-methylhydroxyproline, 3-hydroxybutyrate (BHBA), sphingomyelin (d18:2/16:0, d18:1/16:1), 1-palmitoyl-2-dihomo-linolenoyl-GPC (16:0/20:3n3 or 6), 1-palmitoyl-2-linoleoyl-GPC (16:0/18:2), sphingomyelin (d18:1/20:1, d18:2/20:0), sphingomyelin (d18:1/18:1, d18:2/18:0), 1-(1-enyl-palmitoyl)-2-linoleoyl-GPC (P-16:0/18:2), 1-oleoyl-2-linoleoyl-GPC (18:1/18:2), 2-methylbutyrylcarnitine (C5), and homocysteine. Embodiment 96: The method of embodiment 95, wherein 1-oleoyl-GPS (18:1), pregnen-diol disulfate, dehydroepiandrosterone sulfate (DHEA-S), kynurenine, 1-(1-enyl-palmitoyl)-2-arachidonoyl-GPC (P-16:0/20:4), pregnenetriol disulfate, 1-stearoyl-2-docosahexaenoyl-GPC (18:0/22:6), 1,2-dilinoleoyl-GPC (18:2/18:2), 1-palmitoyl-2-palmitoleoyl-GPC (16:0/16:1), 1-linoleoyl-2-arachidonoyl-GPC (18:2/20:4n6), androsterone sulfate, 1-palmitoyl-2-arachidonoyl-GPC (16:0/20:4n6), androstenediol (3beta,17beta) disulfate (2), 1-stearoyl-2-linoleoyl-GPC (18:0/18:2), 1-palmitoyl-2-docosahexaenoyl-GPC (16:0/22:6), 1-stearoyl-2-arachidonoyl-GPC (18:0/20:4), N-methylhydroxyproline, 3-hydroxybutyrate (BHBA), sphingomyelin (d18:2/16:0, d18:1/16:1), 1-palmitoyl-2-dihomo-linolenoyl-GPC (16:0/20:3n3 or 6), 1-palmitoyl-2-linoleoyl-GPC (16:0/18:2), sphingomyelin (d18:1/20:1, d18:2/20:0), sphingomyelin (d18:1/18:1, d18:2/18:0), 1-(1-enyl-palmitoyl)-2-linoleoyl-GPC (P-16:0/18:2), and 1-oleoyl-2-linoleoyl-GPC (18:1/18:2) are upregulated in ½ EEC and 2-methylbutyrylcarnitine (C5) and homocysteine are down-regulated in ½ EEC. Embodiment 97: The method of embodiment 88, wherein the age is determined by metabolite biomarkers selected from a group comprising N6-methyllysine, nicotinate ribonucleoside, or a combination thereof.

Embodiment 98: A method of monitoring a treatment for endometrial cancer (EC) in a subject in need thereof, the method comprising; a) obtaining a first vaginal swab sample from the subject; b) producing a baseline profile of the vaginal swab sample collected in (a) by detecting at least five or more metabolite biomarkers in the sample obtained in (a); wherein the metabolite biomarkers are selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP); c) administering the treatment for EC to the subject; d) obtaining a second vaginal swab sample from the subject; e) producing a second profile of the vaginal swab sample collected in (d) by detecting at least at least five or more metabolite biomarkers in the sample obtained in (d); wherein the metabolite biomarkers are selected from one or a combination of: N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP); and f) comparing the baseline profile of the vaginal swab sample produced in (b) to the second profile of the vaginal swab sample produced in (e); wherein the treatment is effective if the levels of at least five biomarkers are altered from the baseline profile as compared to the second profile. Embodiment 99: The method of embodiment 98, wherein the method comprises measuring the levels of at least ten or more metabolite biomarkers in the sample obtained. Embodiment 100: The method of embodiment 98 or embodiment 99, wherein the method comprises measuring the levels of at least 15 or more metabolite biomarkers in the sample obtained. Embodiment 101: The method of any one of embodiments 98-100, wherein the method comprises measuring the levels of at least 20 or more metabolite biomarkers in the sample obtained. Embodiment 102: The method of any one of embodiments 98-101, wherein the treatment is effective when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are upregulated and gamma-glutamylglutamine, 1-stearoyl-GPI (18:0), or cytidine 5′-monophosphate (5′-CMP) are downregulated compared to a control profile. Embodiment 103: The method of any one of embodiments 98-102, wherein the metabolite biomarkers are expressed in grade ½ endometrioid endometrial cancer (EEC).

Embodiment 104: The method of any one of embodiments 98-103 wherein producing a profile further comprises characterizing endometrial tumor characteristics comprising tumor size, myometrial invasion, mismatch repair (MMR) status, histological grade, age, or a combination thereof. Embodiment 105: The method of embodiment 104, wherein tumor size is determined by metabolite biomarkers selected from a group comprising 3,7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991. Embodiment 106: The method of embodiment 105, wherein 3,7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991 are down-regulated in tumors greater than or equal to 2 cm. Embodiment 107: The method of embodiment 104, wherein myometrial invasion is determined by metabolite biomarkers selected from a group comprising dihomo-linolenate (20:3n3 or n6), 1-(1-enyl-oleoyl)-GPE (P-18:1)*, 1-(1-enyl-palmitoyl)-GPE (P-16:0)*, 1-stearoyl-GPE (18:0), 1-(1-enyl-palmitoyl)-2-docosahexaenoyl-GPE (P-16:0/22:6)*, 1,2-dilinoleoyl-GPC (18:2/18:2), 1-(1-enyl-palmitoyl)-2-linoleoyl-GPC (P-16:0/18:2)*, 1-stearoyl-GPC (18:0), alpha-tocopherol, 1,2-dipalmitoyl-GPC (16:0/16:0), 1-(1-enyl-palmitoyl)-2-arachidonoyl-GPC (P-16:0/20:4)*, myristoylcarnitine (C14), 1-(1-enyl-palmitoyl)-2-oleoyl-GPC (P-16:0/18:1)*, X-17348, vitamin D3 sulfate, ondansetron, and N-acetylhistamine. Embodiment 108: The method of embodiment 107, wherein dihomo-linolenate (20:3n3 or n6), 1-(1-enyl-oleoyl)-GPE (P-18:1)*, 1-(1-enyl-palmitoyl)-GPE (P-16:0)*, 1-stearoyl-GPE (18:0), 1-(1-enyl-palmitoyl)-2-docosahexaenoyl-GPE (P-16:0/22:6)*, 1,2-dilinoleoyl-GPC (18:2/18:2), 1-(1-enyl-palmitoyl)-2-linoleoyl-GPC (P-16:0/18:2)*, 1-stearoyl-GPC (18:0), alpha-tocopherol, 1,2-dipalmitoyl-GPC (16:0/16:0), 1-(1-enyl-palmitoyl)-2-arachidonoyl-GPC (P-16:0/20:4)*, myristoylcarnitine (C14), 1-(1-enyl-palmitoyl)-2-oleoyl-GPC (P-16:0/18:1)* are upregulated in the absence of myometrial invasion and X-17348, vitamin D3 sulfate, ondansetron, and N-acetylhistamine are down-regulated in the absence of myometrial invasion. Embodiment 109: The method of embodiment 104, wherein mismatch repair (MMR) status is determined by metabolite biomarkers selected from a group comprising lidocaine, 3-methylglutarate/2-methylglutarate, sphingomyelin (d17:1/14:0, d16:1/15:0)*, 4-cholesten-3-one, adenosine, margaroylcarnitine (C17)*, oleoylcarnitine (C18:1), 2-hydroxyadipate, 2,3-dihydroxyisovalerate, 2-isopropylmalate, and sarcosine. Embodiment 110: The method of embodiment 109, wherein are lidocaine, 3-methylglutarate/2-methylglutarate, sphingomyelin (d17:1/14:0, d16:1/15:0)*, 4-cholesten-3-one, adenosine, margaroylcarnitine (C17)*, oleoylcarnitine (C18:1), 2-hydroxyadipate, 2,3-dihydroxyisovalerate, 2-isopropylmalate, and sarcosine down-regulated in MMR proficient cancer. Embodiment 111: The method of embodiment 104, wherein the histological grade is determined by metabolite biomarkers selected from a group comprising X-11308 or 4-hydroxyglutamate. Embodiment 112: The method of embodiment 111, wherein X-11308 is upregulated in ½ EEC and 4-hydroxyglutamate is down-regulated in ½ EEC.

Embodiment 113: A non-invasive method of determining a size of a tumor in a subject with endometrial cancer (EC), the method comprising: determining the patient's levels of five or more metabolites biomarkers by: a) obtaining a cervicovaginal lavage (CVL) sample from the patient; and b) measuring the levels of five or more biomarkers in the sample obtained in (i); wherein the five or more biomarkers comprise, consist of, or consist essentially of: dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, and cytosine; and wherein the size of the tumor is greater than 2 cm if the levels of five or more biomarkers are altered compared to a predetermined threshold. Embodiment 114: The method of embodiment 113, wherein dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) are upregulated in tumors greater than or equal to 2 cm and CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, cytosine are down-regulated in tumors greater than or equal to 2 cm.

Embodiment 115: A non-invasive method of determining a size of a tumor in a subject with endometrial cancer (EC), the method comprising: determining the patient's levels of five or more metabolites biomarkers by: a) obtaining a vaginal swab sample from the patient; and b) measuring the levels of five or more biomarkers in the sample obtained in (i); wherein the five or more biomarkers comprise, consist of, consist essentially of: 7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991; and wherein the size of the tumor is greater than 2 cm if the levels of five or more biomarkers are altered compared to a predetermined threshold. Embodiment 116: The method of embodiment 115, wherein 7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991 are down-regulated in tumors greater than or equal to 2 cm. Embodiment 117: The method of any one of embodiments 113-116, wherein the size of the tumor is greater than 2 cm if the levels of at least ten biomarkers are altered compared to a predetermined threshold. Embodiment 118: The method of any one of embodiments 113-116, wherein the size of the tumor is greater than 2 cm if the levels of at least fifteen biomarkers are altered compared to a predetermined threshold.

Embodiment 119: A non-invasive method of determining a prognosis of endometrial cancer (EC) in a subject in need thereof, the method comprising: a) characterizing endometrial tumor characteristics comprising tumor size, myometrial invasion, mismatch repair (MMR) status, histological grade, age, or a combination thereof by: i) determining the patient's levels of five or more metabolites biomarkers by: 1) obtaining a biological sample from the patient; and 2) measuring the levels of two or more biomarkers in the sample obtained in (1); b) determining the prognosis of the patient; wherein a tumor size larger than 2 cm, presence of myometrial invasion, MMR proficient, and grade 3 is indicative of a poor prognosis and a tumor size smaller than 2 cm, no myometrial invasion, MMR deficient and grade ½ is indicative of a good prognosis. Embodiment 120: The method of embodiment 119, wherein the biological sample comprises a cervicovaginal lavage (CVL) sample, a urine sample, a vaginal swab, or a cervicovaginal secretion; wherein the cervicovaginal secretion is collected via a self collected lavage or a menstrual cup.

Embodiment 121: A non-invasive method of determining a prognosis of endometrial cancer (EC) in a subject in need thereof, the method comprising: a) characterizing endometrial tumor characteristics comprising tumor size, myometrial invasion, mismatch repair (MMR) status, histological grade, age, or a combination thereof by: i) determining the patient's levels of five or more metabolites biomarkers, or a combination thereof by: 1) obtaining a cervicovaginal lavage (CVL) sample from the patient; and 2) measuring the levels of two or more biomarkers in the sample obtained in (i); and b) determining the prognosis of the patient wherein a tumor size larger than 2 cm, presence of myometrial invasion, MMR proficient, and grade 3 is indicative of a poor prognosis and a tumor size smaller than 2 cm, no myometrial invasion, MMR deficient and grade ½ is indicative of a good prognosis.

Embodiment 122: The method of embodiment 121, wherein tumor size is determined by metabolite biomarkers selected from a group comprising dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, and cytosine. Embodiment 123: The method of embodiment 122, wherein dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) are upregulated in tumors greater than or equal to 2 cm and CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, cytosine are down-regulated in tumors greater than or equal to 2 cm.

Embodiment 124: The method of embodiment 121, wherein myometrial invasion is determined by metabolite biomarkers selected from a group comprising ceramide (d18:1/14:0, d16:1/16:0)*, (3′-5′)-cytidylyluridine*, N-stearoyl-sphingosine (d18:1/18:0)*, N-palmitoyl-sphinganine (d18:0/16:0), N-palmitoyl-sphingosine (d18:1/16:0), 1-(1-enyl-palmitoyl)-2-palmitoyl-GPC (P-16:0/16:0)*, alpha-hydroxyisocaproate, CMP, 2′-AMP, AMP, argininate*, 2,3-diphosphoglycerate, cyclic adenosine diphosphate-ribose, histamine, and tryptamine. Embodiment 125: The method of embodiment 124, wherein ceramide (d18:1/14:0, d16:1/16:0)*, (3′-5′)-cytidylyluridine*, N-stearoyl-sphingosine (d18:1/18:0)*, N-palmitoyl-sphinganine (d18:0/16:0), N-palmitoyl-sphingosine (d18:1/16:0), and 1-(1-enyl-palmitoyl)-2-palmitoyl-GPC (P-16:0/16:0)* are upregulated in the in the absence of myometrial invasion and alpha-hydroxyisocaproate, CMP, 2′-AMP, AMP, argininate*, 2,3-diphosphoglycerate, cyclic adenosine diphosphate-ribose, histamine, and tryptamine are down-regulated in the absence of myometrial invasion. Embodiment 126: The method of embodiment 121, wherein mismatch repair (MMR) status is determined by metabolite biomarkers selected from a group comprising decanoylcarnitine (C10), octanoylcarnitine (C8), laurylcarnitine (C12), glutarate (C5-DC), S-adenosylmethionine (SAM), butyrylcarnitine (C4), lyxonate, 6-oxopiperidine-2-carboxylate, adenosine, guanine, and sarcosine. Embodiment 127: The method of embodiment 126, wherein decanoylcarnitine (C10), octanoylcarnitine (C8), and laurylcarnitine (C12) are upregulated in MMR-proficient cancer and glutarate (C5-DC), S-adenosylmethionine (SAM), butyrylcarnitine (C4), lyxonate, 6-oxopiperidine-2-carboxylate, adenosine, guanine, and sarcosine are down-regulated in MMR deficient cancer. Embodiment 128: The method of embodiment 121, wherein the histological grade is determined by metabolite biomarkers selected from a group comprising 1-oleoyl-GPS (18:1), pregnen-diol disulfate, dehydroepiandrosterone sulfate (DHEA-S), kynurenine, 1-(1-enyl-palmitoyl)-2-arachidonoyl-GPC (P-16:0/20:4), pregnenetriol disulfate, 1-stearoyl-2-docosahexaenoyl-GPC (18:0/22:6), 1,2-dilinoleoyl-GPC (18:2/18:2), 1-palmitoyl-2-palmitoleoyl-GPC (16:0/16:1), 1-linoleoyl-2-arachidonoyl-GPC (18:2/20:4n6), androsterone sulfate, 1-palmitoyl-2-arachidonoyl-GPC (16:0/20:4n6), androstenediol (3beta,17beta) disulfate (2), 1-stearoyl-2-linoleoyl-GPC (18:0/18:2), 1-palmitoyl-2-docosahexaenoyl-GPC (16:0/22:6), 1-stearoyl-2-arachidonoyl-GPC (18:0/20:4), N-methylhydroxyproline, 3-hydroxybutyrate (BHBA), sphingomyelin (d18:2/16:0, d18:1/16:1), 1-palmitoyl-2-dihomo-linolenoyl-GPC (16:0/20:3n3 or 6), 1-palmitoyl-2-linoleoyl-GPC (16:0/18:2), sphingomyelin (d18:1/20:1, d18:2/20:0), sphingomyelin (d18:1/18:1, d18:2/18:0), 1-(1-enyl-palmitoyl)-2-linoleoyl-GPC (P-16:0/18:2), 1-oleoyl-2-linoleoyl-GPC (18:1/18:2), 2-methylbutyrylcarnitine (C5), and homocysteine. Embodiment 129: The method of embodiment 128, wherein 1-oleoyl-GPS (18:1), pregnen-diol disulfate, dehydroepiandrosterone sulfate (DHEA-S), kynurenine, 1-(1-enyl-palmitoyl)-2-arachidonoyl-GPC (P-16:0/20:4), pregnenetriol disulfate, 1-stearoyl-2-docosahexaenoyl-GPC (18:0/22:6), 1,2-dilinoleoyl-GPC (18:2/18:2), 1-palmitoyl-2-palmitoleoyl-GPC (16:0/16:1), 1-linoleoyl-2-arachidonoyl-GPC (18:2/20:4n6), androsterone sulfate, 1-palmitoyl-2-arachidonoyl-GPC (16:0/20:4n6), androstenediol (3beta,17beta) disulfate (2), 1-stearoyl-2-linoleoyl-GPC (18:0/18:2), 1-palmitoyl-2-docosahexaenoyl-GPC (16:0/22:6), 1-stearoyl-2-arachidonoyl-GPC (18:0/20:4), N-methylhydroxyproline, 3-hydroxybutyrate (BHBA), sphingomyelin (d18:2/16:0, d18:1/16:1), 1-palmitoyl-2-dihomo-linolenoyl-GPC (16:0/20:3n3 or 6), 1-palmitoyl-2-linoleoyl-GPC (16:0/18:2), sphingomyelin (d18:1/20:1, d18:2/20:0), sphingomyelin (d18:1/18:1, d18:2/18:0), 1-(1-enyl-palmitoyl)-2-linoleoyl-GPC (P-16:0/18:2), and 1-oleoyl-2-linoleoyl-GPC (18:1/18:2) are upregulated in ½ EEC and 2-methylbutyrylcarnitine (C5) and homocysteine are down-regulated in ½ EEC. Embodiment 130: The method of embodiment 121, wherein the age is determined by metabolite biomarkers selected from a group comprising N6-methyllysine, nicotinate ribonucleoside, or a combination thereof.

Embodiment 131: A non-invasive method of determining a prognosis of endometrial cancer (EC) in a subject in need thereof, the method comprising: a) characterizing endometrial tumor characteristics comprising tumor size, myometrial invasion, mismatch repair (MMR) status, histological grade, age, or a combination thereof by: i) determining the patient's levels of two or more metabolites biomarkers by: 1) obtaining a vaginal swab sample from the patient; and 2) measuring the levels of two or more biomarkers in the sample obtained in (i); and b) determining the prognosis of the patient; wherein a tumor size larger than 2 cm, presence of myometrial invasion, MMR proficient, and grade 3 is indicative of a poor prognosis and a tumor size smaller than 2 cm, no myometrial invasion, MMR deficient and grade ½ is indicative of a good prognosis.

Embodiment 132: he method of embodiment 131, wherein tumor size is determined by metabolite biomarkers selected from a group comprising 3,7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991. Embodiment 133: The method of embodiment 132, wherein 3,7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991 are down-regulated in tumors greater than or equal to 2 cm. Embodiment 134: The method of embodiment 131, wherein myometrial invasion is determined by metabolite biomarkers selected from a group comprising dihomo-linolenate (20:3n3 or n6), 1-(1-enyl-oleoyl)-GPE (P-18:1)*, 1-(1-enyl-palmitoyl)-GPE (P-16:0)*, 1-stearoyl-GPE (18:0), 1-(1-enyl-palmitoyl)-2-docosahexaenoyl-GPE (P-16:0/22:6)*, 1,2-dilinoleoyl-GPC (18:2/18:2), 1-(1-enyl-palmitoyl)-2-linoleoyl-GPC (P-16:0/18:2)*, 1-stearoyl-GPC (18:0), alpha-tocopherol, 1,2-dipalmitoyl-GPC (16:0/16:0), 1-(1-enyl-palmitoyl)-2-arachidonoyl-GPC (P-16:0/20:4)*, myristoylcarnitine (C14), 1-(1-enyl-palmitoyl)-2-oleoyl-GPC (P-16:0/18:1)*, X-17348, vitamin D3 sulfate, ondansetron, and N-acetylhistamine. Embodiment 135: The method of embodiment 134, wherein dihomo-linolenate (20:3n3 or n6), 1-(1-enyl-oleoyl)-GPE (P-18:1)*, 1-(1-enyl-palmitoyl)-GPE (P-16:0)*, 1-stearoyl-GPE (18:0), 1-(1-enyl-palmitoyl)-2-docosahexaenoyl-GPE (P-16:0/22:6)*, 1,2-dilinoleoyl-GPC (18:2/18:2), 1-(1-enyl-palmitoyl)-2-linoleoyl-GPC (P-16:0/18:2)*, 1-stearoyl-GPC (18:0), alpha-tocopherol, 1,2-dipalmitoyl-GPC (16:0/16:0), 1-(1-enyl-palmitoyl)-2-arachidonoyl-GPC (P-16:0/20:4)*, myristoylcarnitine (C14), 1-(1-enyl-palmitoyl)-2-oleoyl-GPC (P-16:0/18:1)* are upregulated in the absence of myometrial invasion and X-17348, vitamin D3 sulfate, ondansetron, and N-acetylhistamine are down-regulated in the absence of myometrial invasion. Embodiment 136: The method of embodiment 131, wherein mismatch repair (MMR) status is determined by metabolite biomarkers selected from a group comprising lidocaine, 3-methylglutarate/2-methylglutarate, sphingomyelin (d17:1/14:0, d16:1/15:0)*, 4-cholesten-3-one, adenosine, margaroylcarnitine (C17)*, oleoylcarnitine (C18:1), 2-hydroxyadipate, 2,3-dihydroxyisovalerate, 2-isopropylmalate, and sarcosine. Embodiment 137: The method of embodiment 136, wherein are lidocaine, 3-methylglutarate/2-methylglutarate, sphingomyelin (d17:1/14:0, d16:1/15:0)*, 4-cholesten-3-one, adenosine, margaroylcarnitine (C17)*, oleoylcarnitine (C18:1), 2-hydroxyadipate, 2,3-dihydroxyisovalerate, 2-isopropylmalate, and sarcosine down-regulated in MMR proficient cancer. Embodiment 138: The method of embodiment 131, wherein the histological grade is determined by metabolite biomarkers selected from a group comprising X-11308 or 4-hydroxyglutamate. Embodiment 139: The method of embodiment 138, wherein X-11308 is upregulated in ½ EEC and 4-hydroxyglutamate is down-regulated in ½ EEC.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of monitoring a treatment for endometrial cancer (EC) in a subject in need thereof, the method comprising;
a) obtaining a first cervicovaginal lavage (CVL) sample from the subject;
b) producing a baseline profile of the CVL sample collected in (a) by detecting at least five or more metabolite biomarkers in the sample obtained in (i); wherein the metabolite biomarkers are selected from one or a combination of: 6-oxopiperidine-2-carboxylate, glycerophosphoethanolamine (GPEA), glycerophosphocholine (GPC), guanine, cytosine, glycerophosphoserine, X-19913, X-24724 lyxonate, prolylglycine, glycerophosphoglycerol, or N-acetylserine;
c) administering the treatment for EC to the subject;
d) obtaining a second cervicovaginal lavage (CVL) sample from the subject;
e) producing a second profile of the CVL sample collected in (d) by detecting at least at least five or more metabolite biomarkers in the sample obtained in (i); wherein the metabolite biomarkers are selected from one or a combination of: 6-oxopiperidine-2-carboxylate, GPEA, GPC, guanine, cytosine, glycerophosphoserine, X-19913, X-24724 lyxonate, prolylglycine, glycerophosphoglycerol, or N-acetylserine; and
f) comparing the baseline profile of the CVL sample produced in (b) to the second profile of the CVL sample produced in (e);
wherein the treatment is effective if the levels of at least five biomarkers are altered from the baseline profile as compared to the second profile.

2. The method of claim 1, wherein the treatment is effective when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are upregulated and gamma-glutamylglutamine, 1-stearoyl-GPI (18:0), or cytidine 5′-monophosphate (5′-CMP) are down-regulated.

3. The method of claim 1, wherein producing a profile further comprises characterizing endometrial tumor characteristics comprising tumor size, myometrial invasion, mismatch repair (MMR) status, histological grade, age, or a combination thereof.

4. The method of claim 3, wherein tumor size is determined by metabolite biomarkers selected from a group comprising dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, and cytosine.

5. The method of claim 3, wherein dihomolinolenate (20:3n3 or 3n6), (3′-5′)-adenylyluridine, ceramide (d18:1/14:0, d16:1/16:0), N-palmitoyl-sphingadienine (d18:2/16:0), (3′-5′)-cytidylyluridine, (3′-5′)-guanylylcytidine, bilirubin degradation product, C17H18N2O4 (2), N-stearoyl-sphingosine (d18:1/18:0), (3′-5′)-adenylylcytidine, (3′-5′)-guanylyluridine, dihomolinoleate (20:2n6), 3-hydroxybutyrate (BHBA), N-palmitoyl-sphingosine (d18:1/16:0), 1-(1-enyl-palmitoyl)-2-palmitoyl-GPC (P-16:0/16:0), biliverdin, N-palmitoyl-sphinganine (d18:0/16:0), N6-methyladenosine, 1-stearoyl-GPI (18:0), lactosyl-N-nervonoyl-sphingosine (d18:1/24:1), 1-(1-enyl-palmitoyl)-2-linoleoyl-GPC (P-16:0/18:2), 1-(1-enyl-palmitoyl)-2-arachidonoyl-GPC (P-16:0/20:4), 1-oleoyl-GPC (18:1), ceramide (d18:1/17:0, d17:1/18:0), myristoylcarnitine (C14), 1-(1-enyl-stearoyl)-GPE (P-18:0), 1-dihomo-linolenylglycerol (20:3), 1-palmitoyl-GPC (16:0), N-acetylaspartate (NAA), 1-stearoyl-2-oleoyl-GPS (18:0/18:1), 1-stearoyl-GPS (18:0)*, erucate (22:1n9), behenoyl dihydrosphingomyelin (d18:0/22:0)*, 2-palmitoyl-GPC* (16:0)*, palmitoleoylcarnitine (C16:1)*, 1-(1-enyl-stearoyl)-2-oleoyl-GPE (P-18:0/18:1), 1-stearoyl-2-oleoyl-GPE (18:0/18:1), 1-(1-enyl-palmitoyl)-2-arachidonoyl-GPE (P-16:0/20:4)*, lactosyl-N-palmitoyl-sphingosine (d18:1/16:0), palmitoylcarnitine (C16), N-acetyltaurine, 3-hydroxypalmitoylcarnitine, 1-linoleoylglycerol (18:2), 2′-deoxyuridine, 1-oleoyl-GPE (18:1), N-stearoyl-sphinganine (d18:0/18:0)*, eicosenoate (20:1n9 or in11), 1-myristoyl-2-palmitoyl-GPC (14:0/16:0), sphingomyelin (d18:2/24:1, d18:1/24:2)*, 1-(1-enyl-palmitoyl)-2-oleoyl-GPE (P-16:0/18:1)*, 3-methyl-2-oxovalerate, 1-palmitoyl-2-stearoyl-GPC (16:0/18:0), 1-(1-enyl-palmitoyl)-2-oleoyl-GPC (P-16:0/18:1)*, 1-palmitoyl-GPE (16:0) are upregulated in tumors greater than or equal to 2 cm and CDP-choline, CDP-ethanolamine, NAD+, val-val-ala, 4-hydroxyphenylacetylglutamine, 1-methylguanidine, 2-hydroxy-4-(methylthio)butanoic acid, cysteine, adenosine, AMP, glycerophosphoglycerol, homocysteine, N-acetylcysteine, adenine, cytidine diphosphate, 2,3-diphosphoglycerate, glutathione, reduced (GSH), histidylalanine, Isobar: hexose diphosphates, dihydroorotate, carnosine, valylglutamine, tyrosylglycine, argininate, cytosine are down-regulated in tumors greater than or equal to 2 cm.

6. The method of claim 3, wherein myometrial invasion is determined by metabolite biomarkers selected from a group comprising ceramide (d18:1/14:0, d16:1/16:0)*, (3′-5′)-cytidylyluridine*, N-stearoyl-sphingosine (d18:1/18:0)*, N-palmitoyl-sphinganine (d18:0/16:0), N-palmitoyl-sphingosine (d18:1/16:0), 1-(1-enyl-palmitoyl)-2-palmitoyl-GPC (P-16:0/16:0)*, alpha-hydroxyisocaproate, CMP, 2′-AMP, AMP, argininate*, 2,3-diphosphoglycerate, cyclic adenosine diphosphate-ribose, histamine, and tryptamine; wherein ceramide (d18:1/14:0, d16:1/16:0)*, (3′-5′)-cytidylyluridine*, N-stearoyl-sphingosine (d18:1/18:0)*, N-palmitoyl-sphinganine (d18:0/16:0), N-palmitoyl-sphingosine (d18:1/16:0), and 1-(1-enyl-palmitoyl)-2-palmitoyl-GPC (P-16:0/16:0)* are upregulated in the in the absence of myometrial invasion and alpha-hydroxyisocaproate, CMP, 2′-AMP, AMP, argininate*, 2,3-diphosphoglycerate, cyclic adenosine diphosphate-ribose, histamine, and tryptamine are down-regulated in the absence of myometrial invasion.

7. The method of claim 3, wherein mismatch repair (MMR) status is determined by metabolite biomarkers selected from a group comprising decanoylcarnitine (C10), octanoylcarnitine (C8), laurylcarnitine (C12), glutarate (C5-DC), S-adenosylmethionine (SAM), butyrylcarnitine (C4), lyxonate, 6-oxopiperidine-2-carboxylate, adenosine, guanine, and sarcosine; wherein decanoylcarnitine (C10), octanoylcarnitine (C8), and laurylcarnitine (C12) are upregulated in MMR-proficient cancer and glutarate (C5-DC), S-adenosylmethionine (SAM), butyrylcarnitine (C4), lyxonate, 6-oxopiperidine-2-carboxylate, adenosine, guanine, and sarcosine are down-regulated in MMR deficient cancer.

8. The method of claim 3, wherein the histological grade is determined by metabolite biomarkers selected from a group comprising 1-oleoyl-GPS (18:1), pregnen-diol disulfate, dehydroepiandrosterone sulfate (DHEA-S), kynurenine, 1-(1-enyl-palmitoyl)-2-arachidonoyl-GPC (P-16:0/20:4), pregnenetriol disulfate, 1-stearoyl-2-docosahexaenoyl-GPC (18:0/22:6), 1,2-dilinoleoyl-GPC (18:2/18:2), 1-palmitoyl-2-palmitoleoyl-GPC (16:0/16:1), 1-linoleoyl-2-arachidonoyl-GPC (18:2/20:4n6), androsterone sulfate, 1-palmitoyl-2-arachidonoyl-GPC (16:0/20:4n6), androstenediol (3beta,17beta) disulfate (2), 1-stearoyl-2-linoleoyl-GPC (18:0/18:2), 1-palmitoyl-2-docosahexaenoyl-GPC (16:0/22:6), 1-stearoyl-2-arachidonoyl-GPC (18:0/20:4), N-methylhydroxyproline, 3-hydroxybutyrate (BHBA), sphingomyelin (d18:2/16:0, d18:1/16:1), 1-palmitoyl-2-dihomo-linolenoyl-GPC (16:0/20:3n3 or 6), 1-palmitoyl-2-linoleoyl-GPC (16:0/18:2), sphingomyelin (d18:1/20:1, d18:2/20:0), sphingomyelin (d18:1/18:1, d18:2/18:0), 1-(1-enyl-palmitoyl)-2-linoleoyl-GPC (P-16:0/18:2), 1-oleoyl-2-linoleoyl-GPC (18:1/18:2), 2-methylbutyrylcarnitine (C5), and homocysteine; wherein 1-oleoyl-GPS (18:1), pregnen-diol disulfate, dehydroepiandrosterone sulfate (DHEA-S), kynurenine, 1-(1-enyl-palmitoyl)-2-arachidonoyl-GPC (P-16:0/20:4), pregnenetriol disulfate, 1-stearoyl-2-docosahexaenoyl-GPC (18:0/22:6), 1,2-dilinoleoyl-GPC (18:2/18:2), 1-palmitoyl-2-palmitoleoyl-GPC (16:0/16:1), 1-linoleoyl-2-arachidonoyl-GPC (18:2/20:4n6), androsterone sulfate, 1-palmitoyl-2-arachidonoyl-GPC (16:0/20:4n6), androstenediol (3beta,17beta) disulfate (2), 1-stearoyl-2-linoleoyl-GPC (18:0/18:2), 1-palmitoyl-2-docosahexaenoyl-GPC (16:0/22:6), 1-stearoyl-2-arachidonoyl-GPC (18:0/20:4), N-methylhydroxyproline, 3-hydroxybutyrate (BHBA), sphingomyelin (d18:2/16:0, d18:1/16:1), 1-palmitoyl-2-dihomo-linolenoyl-GPC (16:0/20:3n3 or 6), 1-palmitoyl-2-linoleoyl-GPC (16:0/18:2), sphingomyelin (d18:1/20:1, d18:2/20:0), sphingomyelin (d18:1/18:1, d18:2/18:0), 1-(1-enyl-palmitoyl)-2-linoleoyl-GPC (P-16:0/18:2), and 1-oleoyl-2-linoleoyl-GPC (18:1/18:2) are upregulated in ½ EEC and 2-methylbutyrylcarnitine (C5) and homocysteine are down-regulated in ½ EEC.

9. The method of claim 3, wherein the age is determined by metabolite biomarkers selected from a group comprising N6-methyllysine, nicotinate ribonucleoside, or a combination thereof.

10. A method of monitoring a treatment for endometrial cancer (EC) in a subject in need thereof, the method comprising;
a) obtaining a first vaginal swab sample from the subject;
b) producing a baseline profile of the vaginal swab sample collected in (a) by detecting at least five or more metabolite biomarkers in the sample obtained in (i); wherein the metabolite biomarkers are selected from one or a combination of: N-(2-hydroxypalmitoyl)-sphingosine (d18:1/16:0(2OH)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP);
c) administering the treatment for EC to the subject;
d) obtaining a second vaginal swab sample from the subject;
e) producing a second profile of the vaginal swab sample collected in (d) by detecting at least at least five or more metabolite biomarkers in the sample obtained in (i); wherein the metabolite biomarkers are selected from one or a combination of: N-(2-hydroxypalmitoyl)hy-sphingosine (d18:1/16:0(20H)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP); and
f) comparing the baseline profile of the vaginal swab sample produced in (b) to the second profile of the vaginal swab sample produced in (e);
wherein the treatment is effective if the levels of at least five biomarkers are altered from the baseline profile as compared to the second profile.

11. The method of claim 10, wherein the treatment is effective when N-(2-hydroxypalmitoyl)-sphingosine (d18:1/16:0(20H)), heptadecasphingosine (d17:1), gabapentin, sphingosine, hydroxypalmitoyl sphingomyelin (d18:1/16:0(OH))**, N-palmitoyl-heptadecasphingosine (d17:1/16:0)*, ceramide (d18:1/17:0, d17:1/18:0)*, hexadecasphingosine (d16:1)*, X-17799, sphingadienine, or cholesterol sulfate are upregulated and gamma-glutamylglutamine, 1-stearoyl-GPI (18:0), or cytidine 5′-monophosphate (5′-CMP) are downregulated compared to a control profile.

12. The method of claim 10, wherein producing a profile further comprises characterizing endometrial tumor characteristics comprising tumor size, myometrial invasion, mismatch repair (MMR) status, histological grade, age, or a combination thereof.

13. The method of claim 12, wherein tumor size is determined by metabolite biomarkers selected from a group comprising 3,7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991; wherein 3,7-dimethylurate, pentose acid, N-acetylvaline, dopamine 3-O-sulfate, 1,7-dimethylurate, 1-ribosyl-imidazoleacetate, gamma-glutamylisoleucine, X-12830, N-acetyltryptophan, X-15486, hydantoin-5-propionate, 5-acetylamino-6-formylamino-3-methyluracil, N-acetyl-1-methylhistidine, 4-methylguaiacol sulfate, X-25105, trimethylamine N-oxide, 2R,3R-dihydroxybutyrate, 6-hydroxyindole sulfate, 5-acetylamino-6-amino-3-methyluracil, p-cresol sulfate, X-12216, 3-indoxyl sulfate, 4-hydroxyhippurate, 3-methoxycatechol sulfate (1), N-acetylcarnosine, phenylacetylglutamate, sucralose, 4-methylcatechol sulfate, X-17348, 2-aminophenol sulfate, X-23662, 1,2,3-benzenetriol sulfate (2), 1-methylguanidine, X-25102, doxylamine, X-17808, histidylalanine, methyl-4-hydroxybenzoate sulfate, argininate, ondansetron, and X-24991 are down-regulated in tumors greater than or equal to 2 cm.

14. The method of claim 12, wherein myometrial invasion is determined by metabolite biomarkers selected from a group comprising dihomo-linolenate (20:3n3 or n6), 1-(1-enyl-oleoyl)-GPE (P-18:1)*, 1-(1-enyl-palmitoyl)-GPE (P-16:0)*, 1-stearoyl-GPE (18:0), 1-(1-enyl-palmitoyl)-2-docosahexaenoyl-GPE (P-16:0/22:6)*, 1,2-dilinoleoyl-GPC (18:2/18:2), 1-(1-enyl-palmitoyl)-2-linoleoyl-GPC (P-16:0/18:2)*, 1-stearoyl-GPC (18:0), alpha-tocopherol, 1,2-dipalmitoyl-GPC (16:0/16:0), 1-(1-enyl-palmitoyl)-2-arachidonoyl-GPC (P-16:0/20:4)*, myristoylcarnitine (C14), 1-(1-enyl-palmitoyl)-2-oleoyl-GPC (P-16:0/18:1)*, X-17348, vitamin D3 sulfate, ondansetron, and N-acetylhistamine; wherein dihomo-linolenate (20:3n3 or n6), 1-(1-enyl-oleoyl)-GPE (P-18:1)*, 1-(1-enyl-palmitoyl)-GPE (P-16:0)*, 1-stearoyl-GPE (18:0), 1-(1-enyl-palmitoyl)-2-docosahexaenoyl-GPE (P-16:0/22:6)*, 1,2-dilinoleoyl-GPC (18:2/18:2), 1-(1-enyl-palmitoyl)-2-linoleoyl-GPC (P-16:0/18:2)*, 1-stearoyl-GPC (18:0), alpha-tocopherol, 1,2-dipalmitoyl-GPC (16:0/16:0), 1-(1-enyl-palmitoyl)-2-arachidonoyl-GPC (P-16:0/20:4)*, myristoylcarnitine (C14), 1-(1-enyl-palmitoyl)-2-oleoyl-GPC (P-16:0/18:1)* are upregulated in the absence of myometrial invasion and X-17348, vitamin D3 sulfate, ondansetron, and N-acetylhistamine are down-regulated in the absence of myometrial invasion.

15. The method of claim 12, wherein mismatch repair (MMR) status is determined by metabolite biomarkers selected from a group comprising lidocaine, 3-methylglutarate/2-methylglutarate, sphingomyelin (d17:1/14:0, d16:1/15:0)*, 4-cholesten-3-one, adenosine, margaroylcarnitine (C17)*, oleoylcarnitine (C18:1), 2-hydroxyadipate, 2,3-dihydroxyisovalerate, 2-isopropylmalate, and sarcosine; wherein are lidocaine, 3-methylglutarate/2-methylglutarate, sphingomyelin (d17:1/14:0, d16:1/15:0)*, 4-cholesten-3-one, adenosine, margaroylcarnitine (C17)*, oleoylcarnitine (C18:1), 2-hydroxyadipate, 2,3-dihydroxyisovalerate, 2-isopropylmalate, and sarcosine down-regulated in MMR proficient cancer.

16. The method of claim 12, wherein the histological grade is determined by metabolite biomarkers selected from a group comprising X-11308 or 4-hydroxyglutamate; wherein X-11308 is upregulated in ½ EEC and 4-hydroxyglutamate is down-regulated in ½ EEC.

17. A method comprising:
a) obtaining a vaginal swab sample from a patient;
b) producing a profile of the vaginal swab sample collected in (a) by detecting at least five or more metabolite biomarkers selected from the group consisting of N-(2-hydroxypalmitoyl)-sphingosine (d18:1/16:0(2OH)), heptadecasphingosine (d17:1), gabapentin, sphingosine, hydroxypalmitoyl sphingomyelin (d18:1/16:0(OH)), N-palmitoyl-heptadecasphingosine (d17:1/16:0), ceramide (d18:1/17:0, d17:1/18:0)*, hexadecasphingosine (d16:1)*, X-17799, sphingadienine, cholesterol sulfate, gamma-glutamylglutamine, 1-stearoyl-GPI (18:0), or cytidine 5′-monophosphate (5′-CMP);
c) analyzing the vaginal swab sample profile produced in (b).

18. The method of embodiment 17, wherein producing a profile comprises detecting at least ten or more biomarkers.

19. The method of embodiment 17, wherein the method predicts the risk of endometrial hyperplasia or cancer in women.

20. The method of embodiment 17, wherein the method diagnoses endometrial cancer in women.
